(19) United States
(12) Patent Application Publication
(10) Pub. No.: US 2021/0379178 A1
(43) Pub. Date: Dec. 9, 2021

(54) COMPOSITIONS COMPRISING HIV ENVELOPES TO INDUCE HIV-1 ANTIBODIES

(71) Applicant: Duke University, Durham, NC (US)

(72) Inventors: Kevin SAUNDERS, Durham, NC (US); Barton F. HAYNES, Durham, NC (US); Kevin J. WIEHE, Durham, NC (US)

(21) Appl. No.: 17/281,933

(22) PCT Filed: Sep. 4, 2019
(86) PCT No.: PCT/US19/49431

§ 371 Date: Mar. 31, 2021

Related U.S. Application Data
(60) Provisional application No. 62/739,701, filed on Oct. 1, 2018.

(51) Int. Cl. A61K 39/12; C12N 15/63; C07K 14/16; C12N 7/00

(52) U.S. Cl. CPC A61K 39/12; C12N 15/63; C12N 2740/16134; C12N 7/00; C12N 2740/16122; C07K 14/162

(57) ABSTRACT

The invention is directed to modified HIV-1 envelopes, compositions comprising these modified envelopes, nucleic acids encoding these modified envelopes, compositions comprising these nucleic acids, and methods of using these modified HIV-1 envelopes and/or these nucleic acids to induce immune responses.

This application claims the benefit and priority of U.S. Application Ser. No. 62/739,701 filed Oct. 1, 2018, which content is incorporated by reference in its entirety.

This invention was made with government support under Center for HIV/AIDS Vaccine Immunology-Immunogen Design grant UM1-AI100645 from the NIH, NIAID, Division of AIDS. The government has certain rights in the invention.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and methods for induction of an immune response, for example cross-reactive (broadly) neutralizing (bn) Ab induction. In certain embodiments, the methods use compositions comprising HIV-1 envelope immunogens designed to bind to precursors, and/or unmutated common ancestors (UCAs) of different HIV-1 bnAbs. In certain embodiments, these are UCAs of V1V2 glycan and V3 glycan binding antibodies. Thus, in certain embodiments the invention provides HIV-1 envelope immunogen designs with multimerization and variable region sequence optimization for enhanced UCA-targeting. In certain embodiments the invention provides HIV-1 envelope immunogen designs with multimerization and variable region sequence optimization for enhanced targeting and inductions of multiple antibody lineages, e.g. but not limited to V3 lineage, V1V2 lineages of antibodies.

In certain aspects the invention provides compositions comprising a selection of HIV-1 envelopes and/or nucleic acids encoding these envelopes as described herein for example but not limited to designs as described herein. Without limitations, these selected combinations comprise envelopes which provide representation of the sequence (genetic) and antigenic diversity of the HIV-1 envelope variants which lead to the induction of V1V2 glycan and V3 glycan antibody lineages.

In certain aspects the invention provides a recombinant HIV-1 envelope comprising a 17 amino acid (17aa) V1 region, lacking glycosylation at position N133 and N138 (HXB2 numbering), comprising glycosylation at N301 (HXB2 numbering) and N332 (HXB2 numbering), comprising modifications wherein glycan holes are filled (D230N_H289N_P291S (HXB2 numbering)), comprising the “GDIR” or “GDIK” motif at the position corresponding to the amino acid changes #3 in the sequences depicted in FIG. 8B, or any trimer stabilization modifications, UCA targeting modification, immunogenicity modification, or combinations thereof, for example but not limited to these described in Table 2, FIG. 8B (amino acid changes numbered 1-5), and/or FIGS. 21-25. In certain embodiments the recombinant envelope optionally comprises any combinations of these modifications.

In certain embodiments, the recombinant HIV-1 envelope binds to precursors, and/or UCAs of different HIV-1 bnAbs. In certain embodiments, these are UCAs of V1V2 glycan and V3 glycan antibodies. In certain embodiments the envelope is 19CV3. In certain embodiments the envelope is any one of the envelopes listed in Table 1, Table 2 or FIGS. 21-25. In certain embodiments, the envelope is not CH848 10.17 DT variant described previously in WO2018/161049.

In certain embodiments the envelope is a protomer which could be comprised in a stable trimer.

In certain embodiments the envelope comprises additional mutations stabilizing the envelope trimer. In certain embodiments these including but are not limited to SOSIP mutations. In certain embodiments mutations are selected from sets F1-F14, VT1-VT8 mutations described herein, or any combination or subcombination within a set. In certain embodiments, the selected mutations are F14. In other embodiments, the selected mutations are VT8. In certain embodiments, the selected mutations are F4 and VT8 combined.

In certain embodiments, the invention provides a recombinant HIV-1 envelope of FIG. 1, FIG. 2, FIG. 3, or FIGS. 21-25. In certain embodiments, the invention provides a nucleic acid encoding any of the recombinant envelopes. In certain embodiments, the nucleic acids comprise an mRNA formulated for use as a pharmaceutical composition.

In certain embodiments the inventive designs comprise specific changes ((D230N_H289N_P291S HXB2 numbering)), as shown in FIG. 21, which fill glycan holes with the introduction of new glycosylation sites to prevent the binding of strain-specific antibodies that could hinder broad neutralizing antibody development (Wagh, Kshitij et al. “Completeness of HIV-1 Envelope Glycan Shield at Transmission Determines Neutralization Breadth.” Cell reports vol. 25, 4 (2018): 893-908.e7. doi:10.1016/j.celrep.2018.09.087; Crooks, Ema T et al. “Vaccine-Elicited Tier 2 HIV-1 Neutralizing Antibodies Bind to Quaternary Epitopes Involving Glycan-Deficient Patches Proximal to the CD4 Binding Site.” PLoS pathogens vol. 11, 5 e1004932. 29 May. 2015, doi:10.1371/journal.ppat.1004932)

In certain embodiments, the inventive designs comprise modifications, including without limitation fusion of the HIV-1 envelope with ferritin using linkers between the HIV-1 envelope and ferritin designed to optimize ferritin nanoparticle assembly.

In certain embodiments, the invention provides HIV-1 envelopes comprising Lys327 (HXB2 numbering) optimized for administration as a prime to initiate V3 glycan antibody lineage, e.g. DH270 antibody lineage.

In certain embodiments, the invention provides HIV-1 envelopes comprising Lys169 (HXB2 numbering).

In certain embodiments, the invention provides a composition comprising any one of the inventive envelopes or nucleic acid sequences encoding the same. In certain embodiments, the nucleic acid is mRNA. In certain embodiments, the mRNA is comprised in a lipid nano-particle (LNP).

In certain embodiments, the invention provides compositions comprising a nanoparticle which comprises any one of the envelopes of the invention.

In certain embodiments, the invention provides compositions comprising a nanoparticle which comprises any one of the envelopes of the invention, wherein the nanoparticle is a ferritin self-assembling nanoparticle.

In certain embodiments, the invention provides a method of inducing an immune response in a subject comprising administering an immunogenic composition comprising any one of the stabilized recombinant HIV-1 envelopes of the invention. In certain embodiments, the composition is administered as a prime and/or a boost. In certain embodiments, the composition comprises nanoparticles. In certain embodiments, methods of the invention further comprise administering an adjuvant.

In certain embodiments, the invention provides a composition comprising a plurality of nanoparticles comprising a plurality of the recombinant HIV-1 envelopes/trimers of the invention. In non-limiting embodiments, the envelopes/trimers of the invention are multimeric when comprised in a nanoparticle. The nanoparticle size is suitable for delivery. In non-liming embodiments the nanoparticles are ferritin based nanoparticles.

### BRIEF DESCRIPTION OF THE DRAWINGS

FIG. 1 shows non-limiting embodiments of nucleic acid sequences of envelopes of the invention.

FIG. 2 shows non-limiting embodiments of amino acid sequences of envelopes of the invention.

FIG. 3 shows non-limiting embodiments of the sortase design of an envelope of the invention.

FIG. 4 shows that CH0848 10.17DT SOSIP engages the DH270 UCA Fab with 60 nM affinity.

FIG. 5 shows natural envelopes with 17 aa V1 loops lacking N133/N138 glycans exist in vivo.

FIG. 6 shows CH0848.D1305.10.19, and CH0848.D949.10.17 V1V2 loop alignment and that CH0848.D1305.10.19 lacks N133 and N138 glycans in the V1 region of HIV-1 Env.

FIG. 7 shows DH270 UCA does not bind natural Env CH0848.D1305.10.19 that has a 17 aa V1 loop and lacks N133 and N138 glycans.

FIGS. 8A and 8B show that the CH0848 natural Env with a 17 aa V1 loop and no N133 and N138 glycan has eliminated the N295, N301, and N332 glycan. The figure shows JRFL, CH0848.D1305.10.19, and CH0848.D949.10.17 V3 loop alignment.

FIGS. 9A and 9B show that the DH270-resistant CH0848 natural Env with a 17 aa V1 loop and no N133 and N138 glycan acquire V2 apex bnAb binding Potential V3-glycan escape variant is recognized by V2 apex bnAbs.

FIG. 10 shows CH0848.D1305.10.19, and CH0848.D949.10.17 V2 loop alignment and that CH0848.D949.10.17 clone encodes E169 instead of K169. K169E mutations are known to eliminate binding of V1V2 glycan bnAbs.

FIG. 11 shows the design of V3 chimeric CH0848 Envelope antigenic for V1V2 glycan and V3 glycan.

FIG. 12 shows that 19CV3 binds to UCAs of V1V2 glycan and V3 glycan antibodies.

FIG. 13 shows non-limiting embodiments of prime boost regimens combining germline targeting and B cell mosaic Envs.

FIG. 14 shows biolayer interferometry binding by different members of the DH270 V3-glycan antibody lineage. The precursor of the lineage is DH270 UCA3. Somatically mutated lineage members (DH270UCA3 is the unmutated common ancestor, DH270 14, DH270.1 and DH270.6 have increasing somatic mutations) bind better to Arg327 than Lys327. The germline precursor requires Lys327 in order to bind and stay bound to CH848CH848.3.D0949.10.17 N133D N138T D230N_H289N_P219S DS.SOSIP gp140 trimer.

FIGS. 15A-B shows that the addition of E169K enables binding of V1V2-glycan broadly neutralizing antibody PGT145 while retaining V3-glycan antibody binding. Antibody binding was measured by biolayer interferometry. The red vertical line demarks the change from association phase to dissociation phase. Binding curves to CH848.D949.10.17_N133D/N138T is shown in FIG. 15A and CH848.D949.10.17_N133D/N138T/E169K is shown in FIG. 15B. Antibody DH542 is the same as antibody DH270.6.

FIGS. 16A-B shows 19CV3 induces serum binding antibody responses in DH270 germline precursor knockin mice. Knockin mice were immunized with CH848.D1305.10.19_D949V3 gp140 trimer plus adjuvant (red, n=6) or adjuvant alone (silver, n=2). Serum antibody binding to the CH848.D1305.10.19_D949V3 Env trimer used for immunization (FIG. 16A) or the gp120 subunit from a related virus (FIG. 16B). Group mean values are shown.

FIGS. 17A-B shows 19CV3 induces serum antibodies that neutralize HIV-1 with and without V1 glycans removed. Serum antibody neutralization of HIV-1 infection of TZM-bl cells. DH270 germline precursor knockin mice were immunized with CH848.D1305.10.19_D949V3 plus adjuvant (circles, n=6) or adjuvant alone (squares, n=2). Serum was tested for neutralization of HIV-1 isolates CH848.D949.10.17 N133D/N138T (FIG. 17A) and CH848.D949.10.17 (FIG. 17B). Neutralization titers are shown as the reciprocal dilution of serum required to inhibit 50% of virus replication. The neutralization titer for the group were averaged as the geometric mean.

FIGS. 18A-B shows vaccine-induced serum HIV-1 antibody responses in CH01 germline precursor knock-in mice. Knock-in mice were immunized with CH848.D1305.10.19_D949V3 (19CV3) plus adjuvant (circles, n=6) or adjuvant alone (squares, n=3). FIG. 18A shows serum antibody binding to the CH848.D1305.10.19_D949V3 Env trimer used for immunization. Group mean values are shown. FIG. 18B shows serum antibody neutralization of HIV-1 infection of TZM-bl cells. Serum was tested for neutralization against three genetically distinct HIV-1 isolates from CRF AG, Glade A, and Glade C. Neutralization titers are shown as the reciprocal dilution of serum required to inhibit 50% of virus replication. The group geometric mean neutralization titer is indicated with a horizontal bar. Serum lacked neutralization of the negative control murine leukemia virus.

FIG. 19 shows CH848.D1305.10.19_D949V3 (19CV3) DS.SOSIP gp140 elicits V3 glycan directed binding antibodies in rhesus macaques. Serum antibodies were examined for binding to CH848 Env trimers with (WT) and without the N332 glycan (N332A) over the course of vaccination. Binding titers were higher for CH848 Env trimers with the N332 glycan present. This is significant because broadly neutralizing antibodies target the N332 glycan and require it for binding to Env trimers. Arrows indicate time of immunization. Mean and standard error are shown for the group of 3 macaques.

FIGS. 20A-B shows vaccination of rhesus macaques with CH848.D1305.10.19_D949V3 (19CV3) DS.SOSIP gp140 elicits glycan-dependent serum neutralizing antibodies. FIG. 20A shows serum neutralization of kifunensine-treated JR-FL or murine leukemia virus. Kifunensine treatment of virus results in Man₉GlcNAc₂ glycosylation of HIV-1 envelope. Neutralization of Man₉GlcNAc₂-enriched virus can suggest the presence of mannose-reactive neutralizing HIV-1 antibodies. DH270 bnAbs require Man₉GlcNAc₂-enrichment for neutralization early in their development, thus serum neutralization of Man₉GlcNAc₂-enriched JR-FL may indicate elicitation of precursors of DH270-like antibodies. FIG. 20B shows serum neutralization of a panel of autologous CH848 viruses and heterologous genetically distinct HIV-1 isolates. Neutralization of JRFL was dependent on Man₉GlcNAc₂-enrichment. Murine leukemia virus was used as a non-HIV negative control for neutralization. Neutralization titers are shown as reciprocal plasma dilution that inhibits 50% of virus replication (ID50). Each symbol represents an individual macaque. Horizontal bars show the group geometric mean (n=3).

FIGS. 21A-B show non-limiting embodiments for sequences of the invention comprising amino acid Arg327 (K327R). In the amino acid sequences (FIG. 21B), underlined is the signal peptide and the preceding four amino acids indicate the cloning site/kozak sequence (VDTA) neither of which that would not be part of the final recombinant protein.

FIGS. 22A-B show non-limiting embodiments of sequences of the invention comprising varying linkers between the envelope and ferritin proteins. In the amino acid sequences (FIG. 22B), underlined is the signal peptide and the preceding four amino acids indicate the cloning site/kozak sequence (VDTA) neither of which that would not be part of the final recombinant protein.

FIGS. 23A-B show non-limited embodiments of designs of 19CV3 sequences. In the amino acid sequences (FIG. 23B), underlined is the signal peptide and the preceding four amino acids indicate the cloning site/kozak sequence (VDTA) neither of which that would not be part of the final recombinant protein.

FIGS. 24 A-B show non-limited embodiments of designs of 19CV3 sequences. Amino acids H66A_A582T_L587A are referred to JS2 or “joe2” mutations. In the amino acid sequences (FIG. 24B), underlined is the signal peptide and the preceding four amino acids indicate the cloning site/kozak sequence (VDTA) neither of which that would not be part of the final recombinant protein.

FIGS. 25A-B show a summary of non-limiting embodiments of envelope designs of the invention.

FIG. 26 shows one embodiment of a design for the production of trimeric HIV-1 Env on ferritin nanoparticles.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

The invention provides methods of using these pan bnAb envelope immunogens.

In certain aspect, the invention provides compositions for immunizations to induce lineages of broad neutralizing antibodies. In certain embodiments, there is some variance in the immunization regimen; in some embodiments, the selection of HIV-1 envelopes may be grouped in various combinations of primes and boosts, either as nucleic acids, proteins, or combinations thereof. In certain embodiments the compositions are pharmaceutical compositions which are immunogenic. In certain embodiments, the compositions comprise amounts of envelopes which are therapeutic and/or immunogenic.

In one aspect the invention provides a composition for a prime boost immunization regimen comprising any one of the envelopes described herein, or any combination thereof wherein the envelope is a prime or boost immunogen. In certain embodiments the composition for a prime boost immunization regimen comprises one or more envelopes described herein.

In certain embodiments, the compositions contemplate nucleic acid, as DNA and/or RNA, or recombinant protein immunogens either alone or in any combination. In certain embodiments, the methods contemplate genetic, as DNA and/or RNA, immunization either alone or in combination with recombinant envelope protein(s).

mRNA

In some embodiments the antigens are nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

The envelope used in the compositions and methods of the invention can be a gp160, gp150, gp145, gp140, gp120, gp41, N-terminal deletion variants as described herein, cleavage resistant variants as described herein, or codon optimized sequences thereof. In certain embodiments the composition comprises envelopes as trimers. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as a particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 85%, 90%, 95% native like trimers.

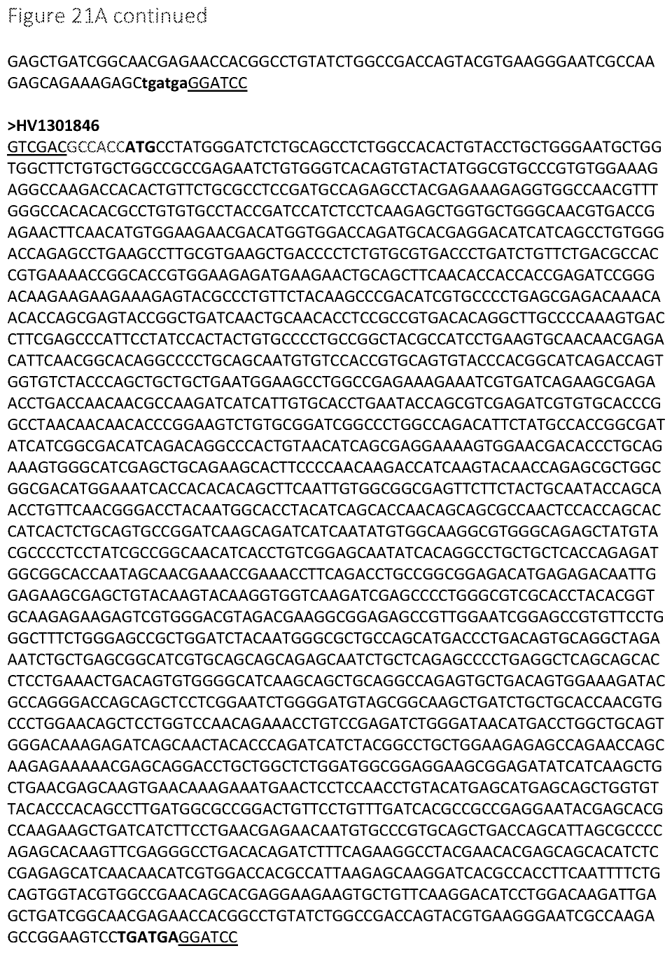

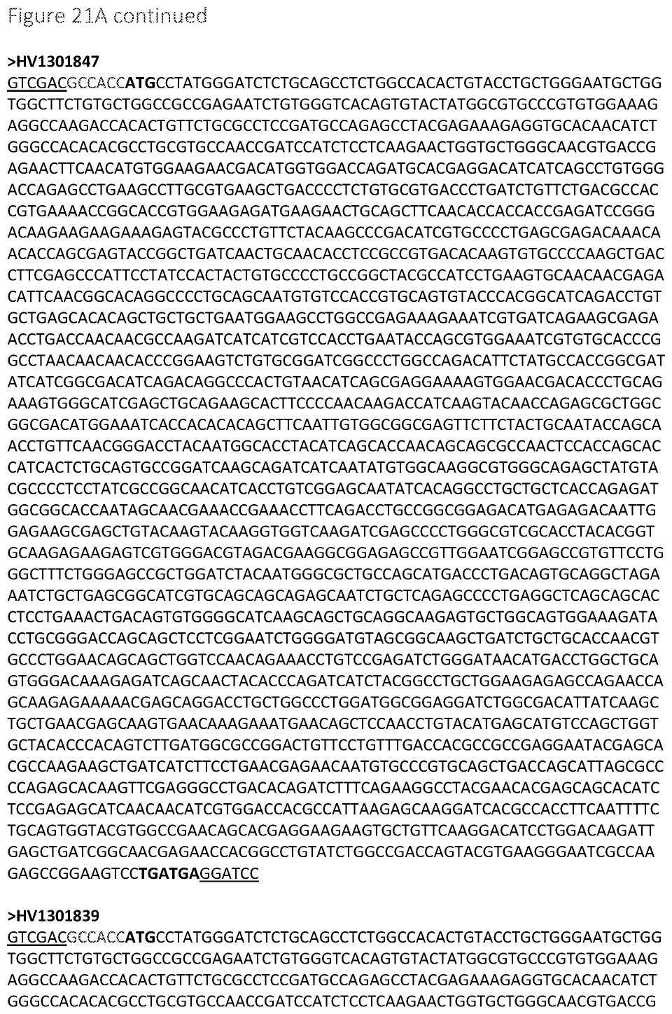

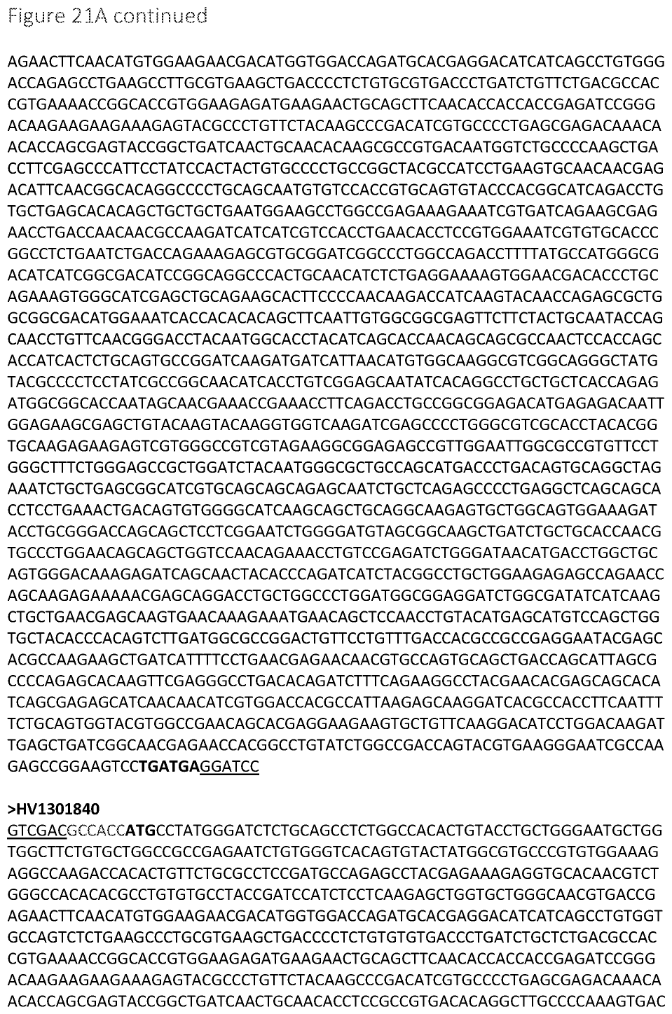

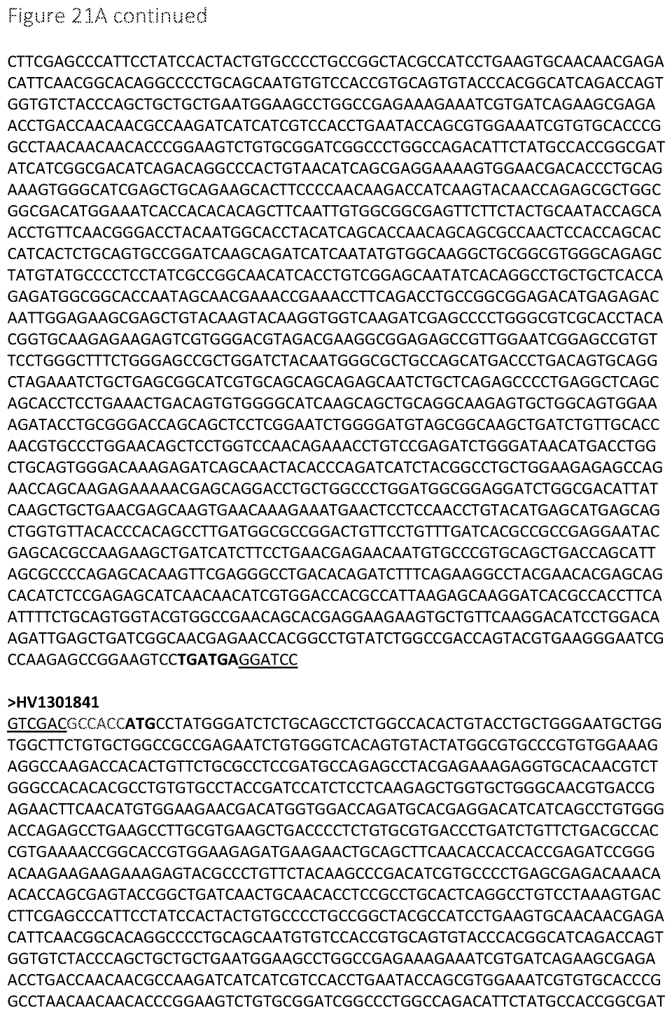

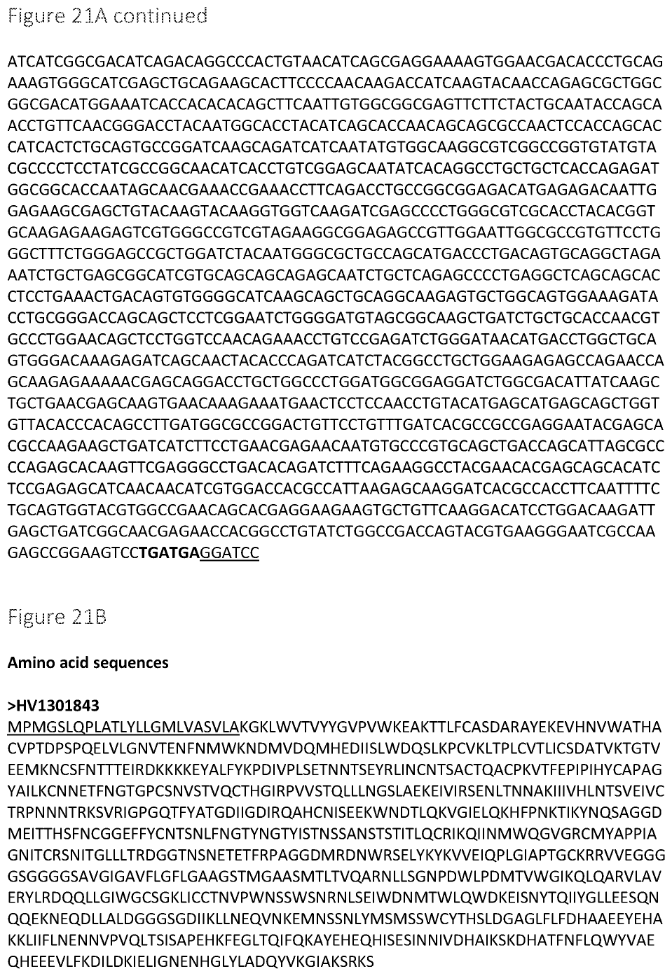

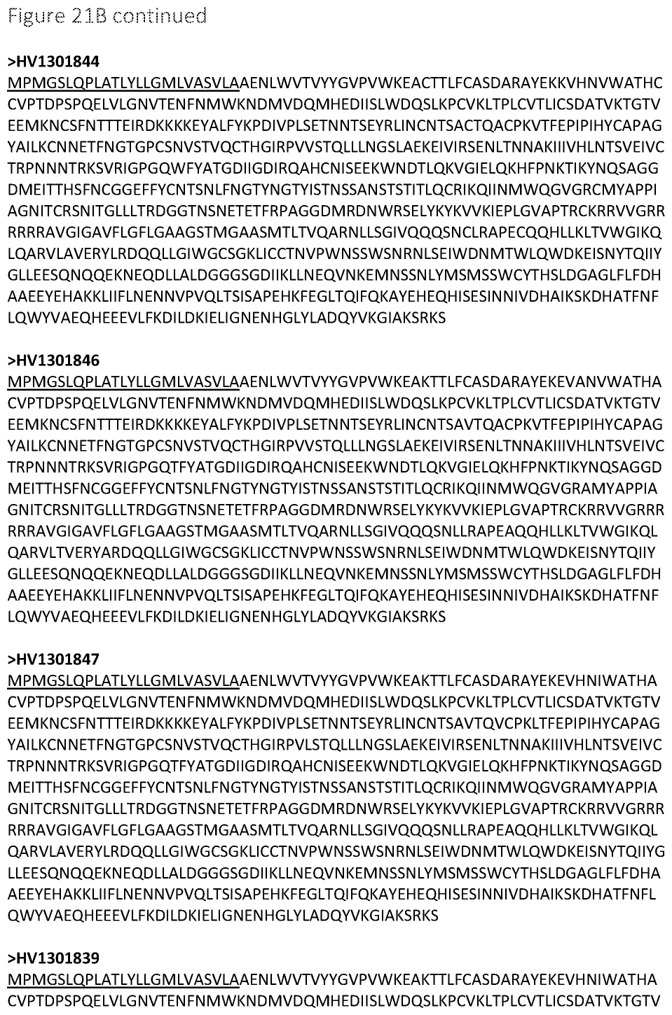

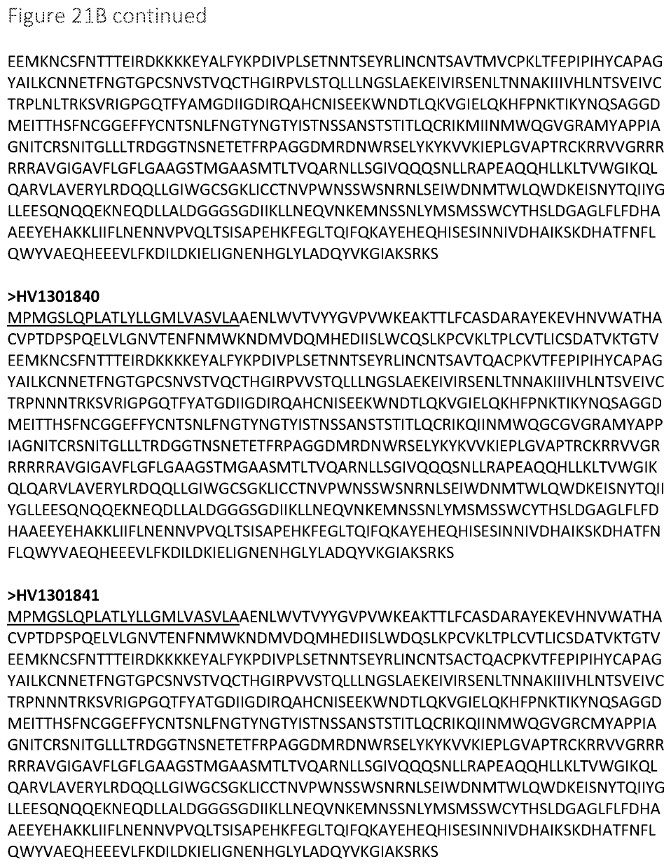

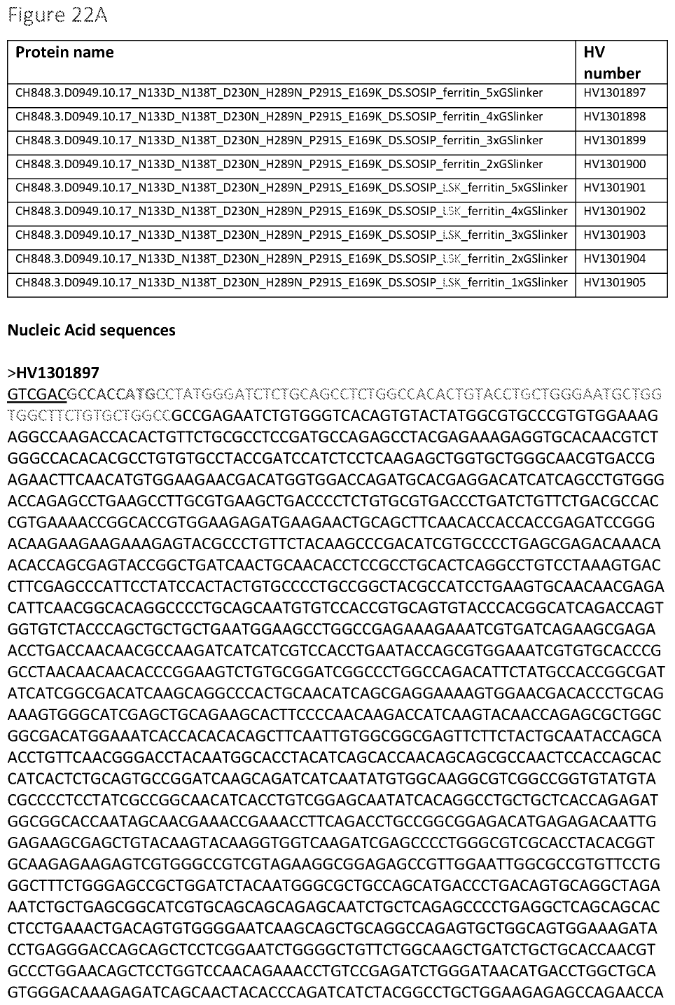

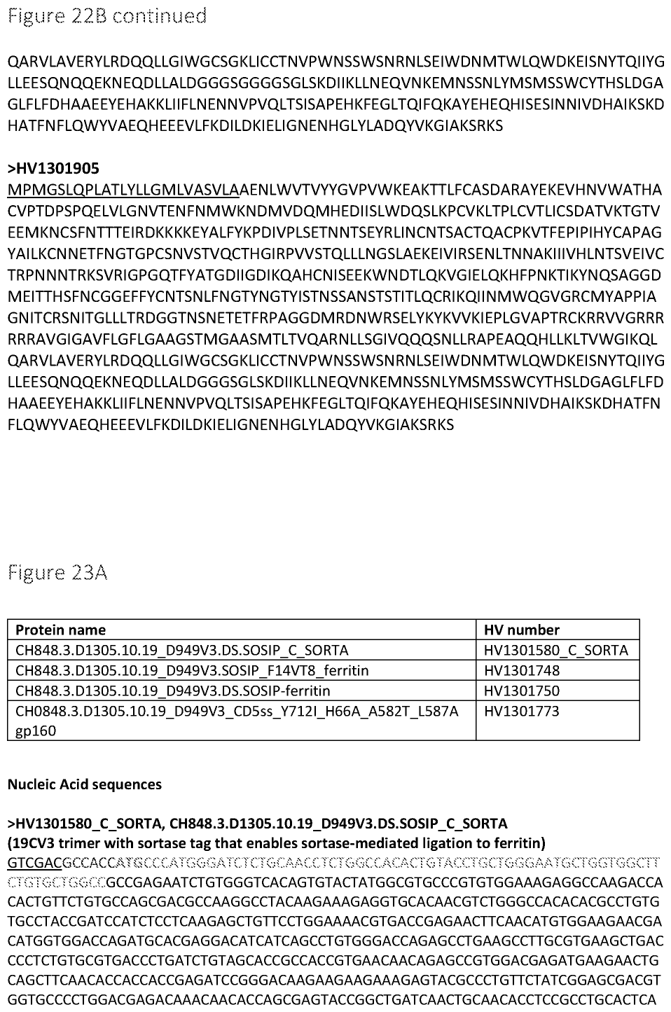

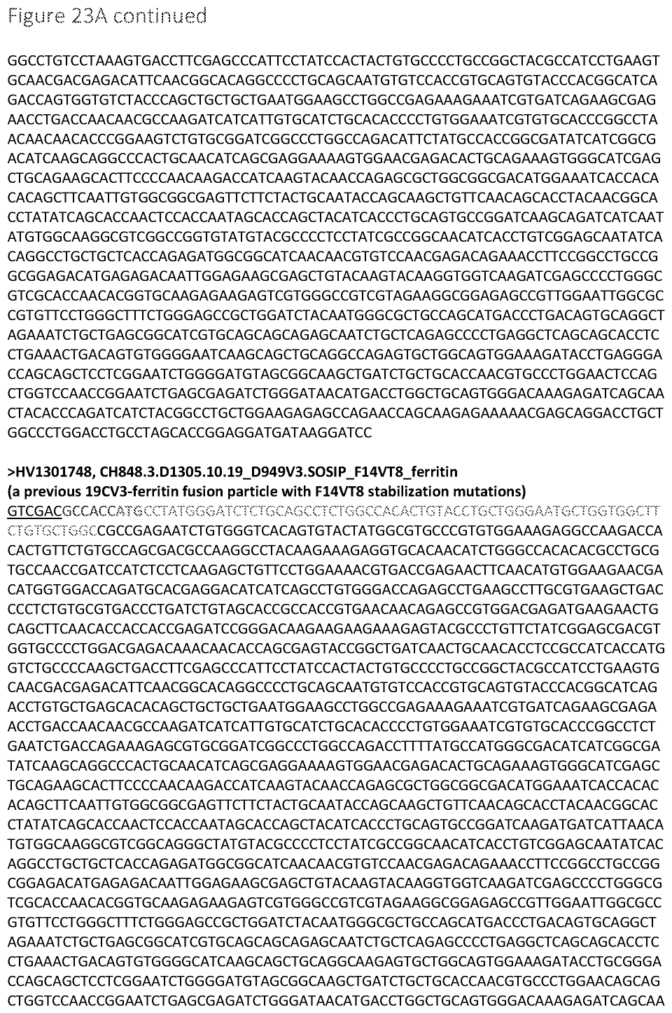

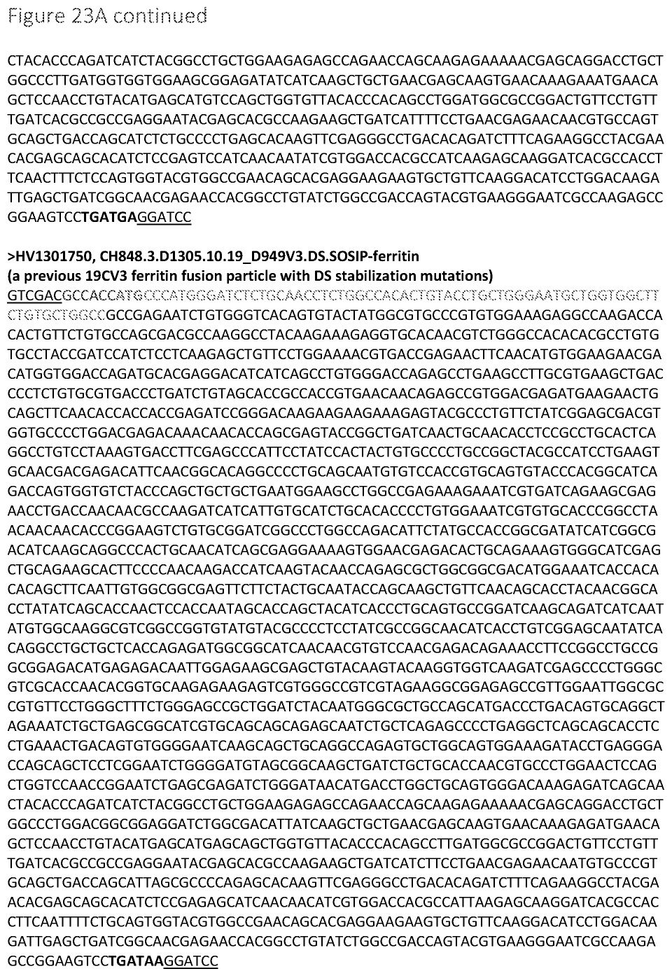

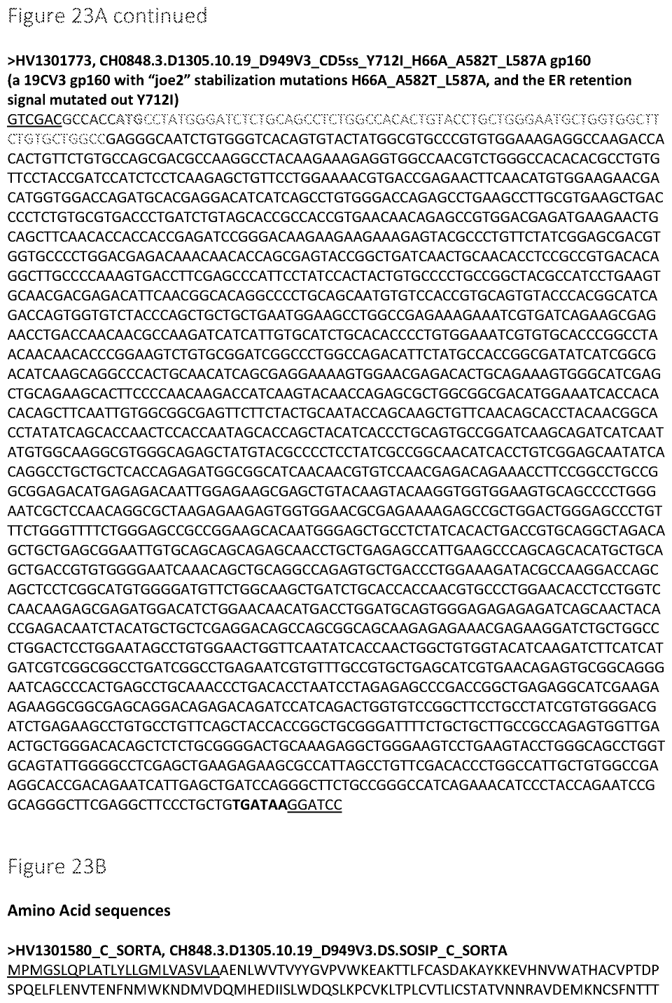

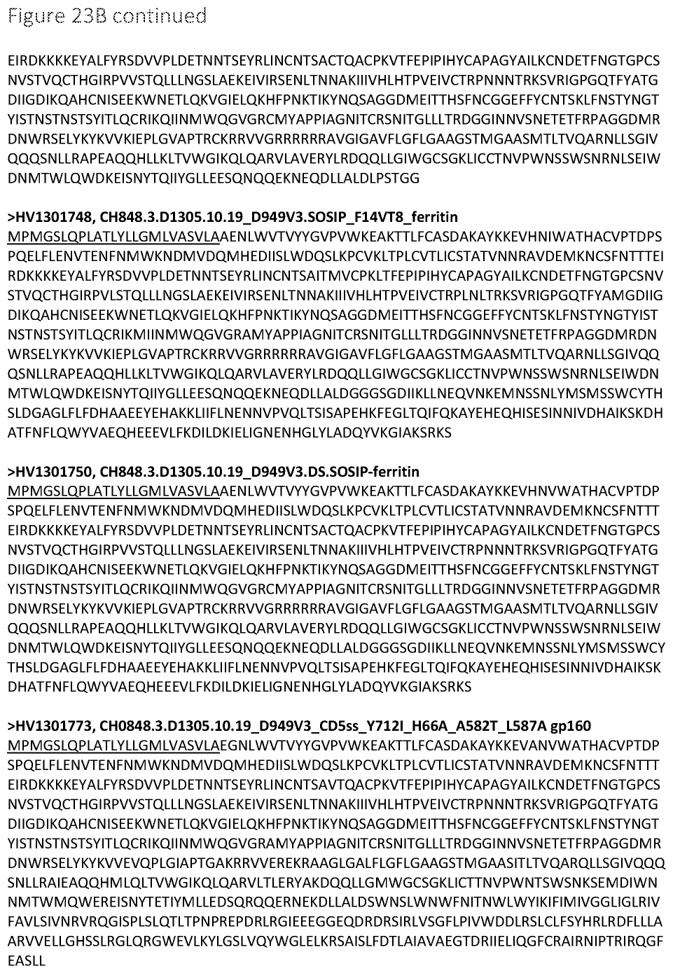

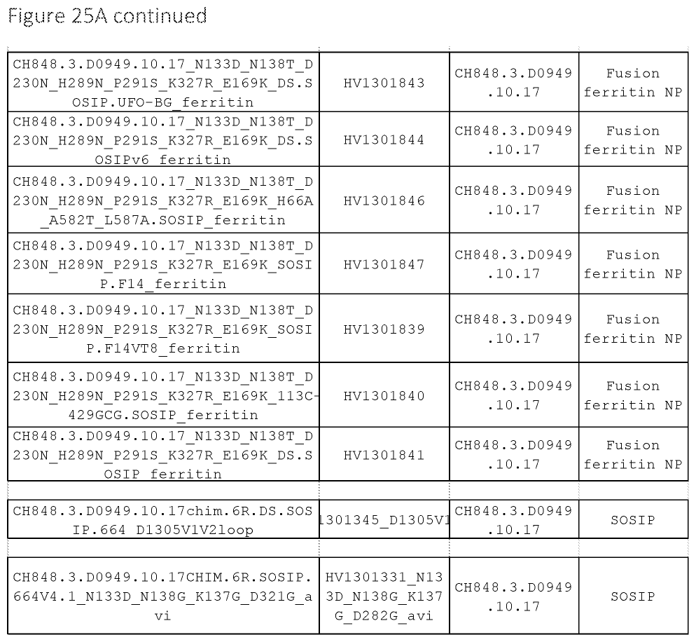

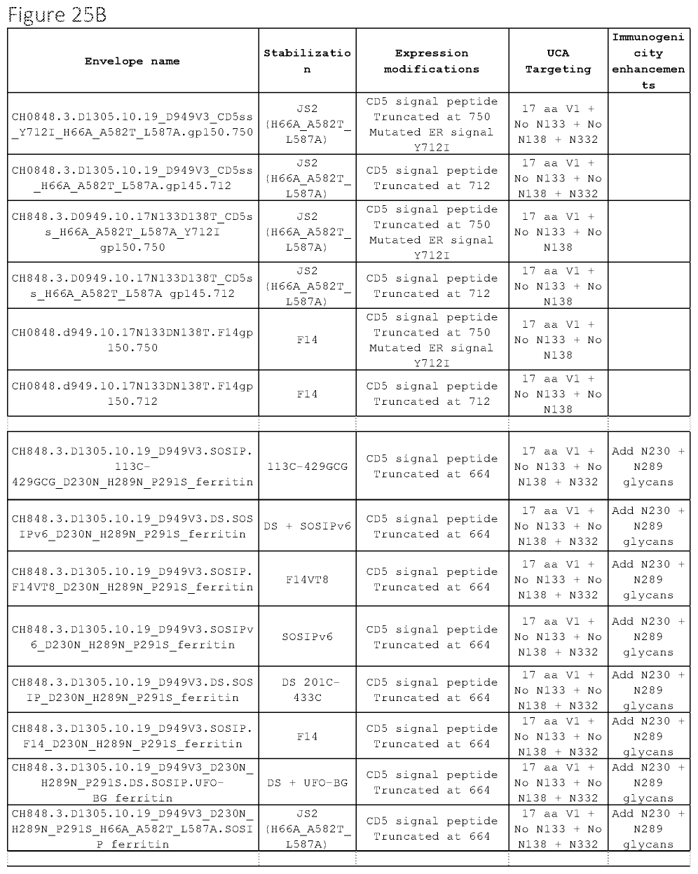

In certain embodiments the envelope is any of the forms of HIV-1 envelope. In certain embodiments the envelope is gp120, gp140, gp145 (i.e. with a transmembrane domain), or gp150. In certain embodiments, gp140 is designed to form a stable trimer. See Table 1, 2, FIGS. 21-25 for non-limiting examples of sequence designs. In certain embodiments envelope protomers form a trimer which is not a SOSIP timer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” and WO2017/151801 which content is herein incorporated by reference in its entirety. In certain embodiments the envelopes of the invention are engineered and comprise non-naturally occurring modifications.

In certain embodiments, the envelope is in a liposome. In certain embodiments the envelope comprises a transmembrane domain with a cytoplasmic tail, wherein the transmembrane domain is embedded in a liposome. In certain embodiments, the nucleic acid comprises a nucleic acid sequence which encodes a gp120, gp140, gp145, gp150, or gp160.

In certain embodiments, where the nucleic acids are operably linked to a promoter and inserted in a vector, the vector is any suitable vector. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, 3M052, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339).

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a stable pool of cells encoding any one of the envelopes of the invention suitable for recombinant expression.

In certain aspects, the invention provides a recombinant HIV-1 envelope polypeptide as described here, wherein the polypeptide is a non-naturally occurring protomer designed to form an envelope trimer. The invention also provides nucleic acids encoding these recombinant polypeptides. Non-limiting examples of amino acids and nucleic acid of such protomers are disclosed herein.

In certain aspects the invention provides a recombinant trimer comprising three identical protomers of an envelope. In certain aspects the invention provides an immunogenic composition comprising the recombinant trimer and a carrier, wherein the trimer comprises three identical protomers of an HIV-1 envelope as described herein. In certain aspects the invention provides an immunogenic composition comprising nucleic acid encoding these recombinant HIV-1 envelope and a carrier.

Sequences/Clones

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41—named as gp140ACFI (gp140CFI), gp140 Envs with the deletion of only the cleavage (C) site and fusion (F) domain—named as gp140ACF (gp140CF), gp140 Envs with the deletion of only the cleavage (C)—named gp140AC (gp140C) (See e.g. Liao et al. Virology 2006,353, 268-282), gp150s, gp41s, can be readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

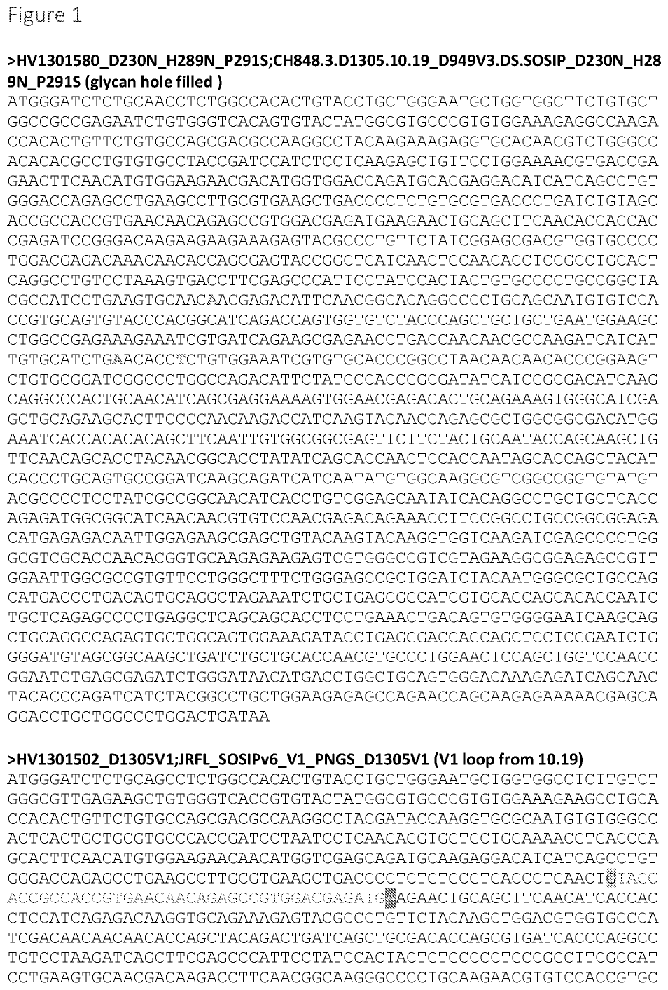

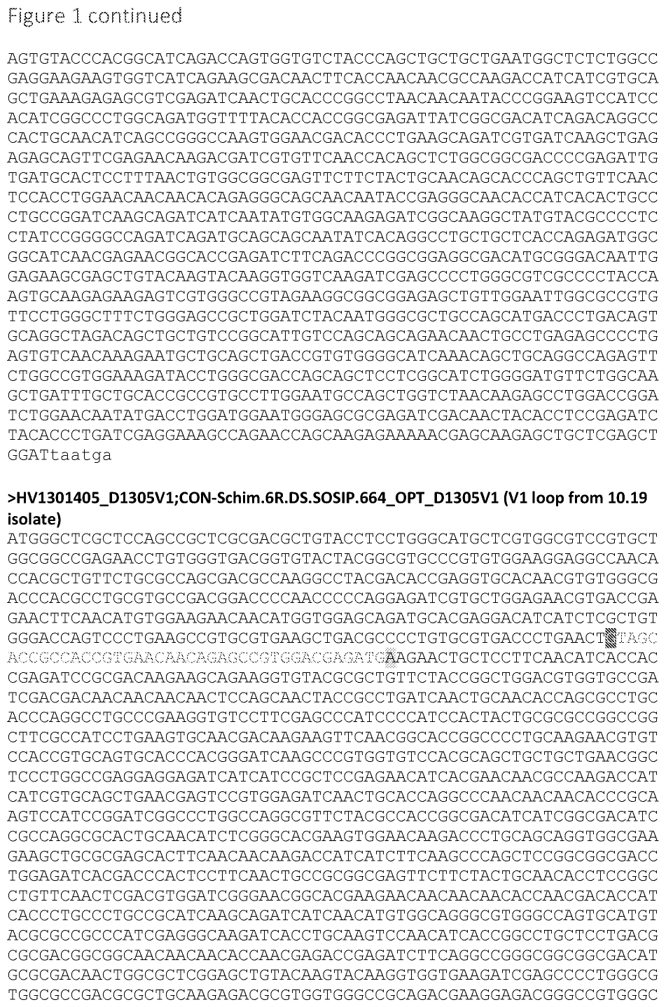

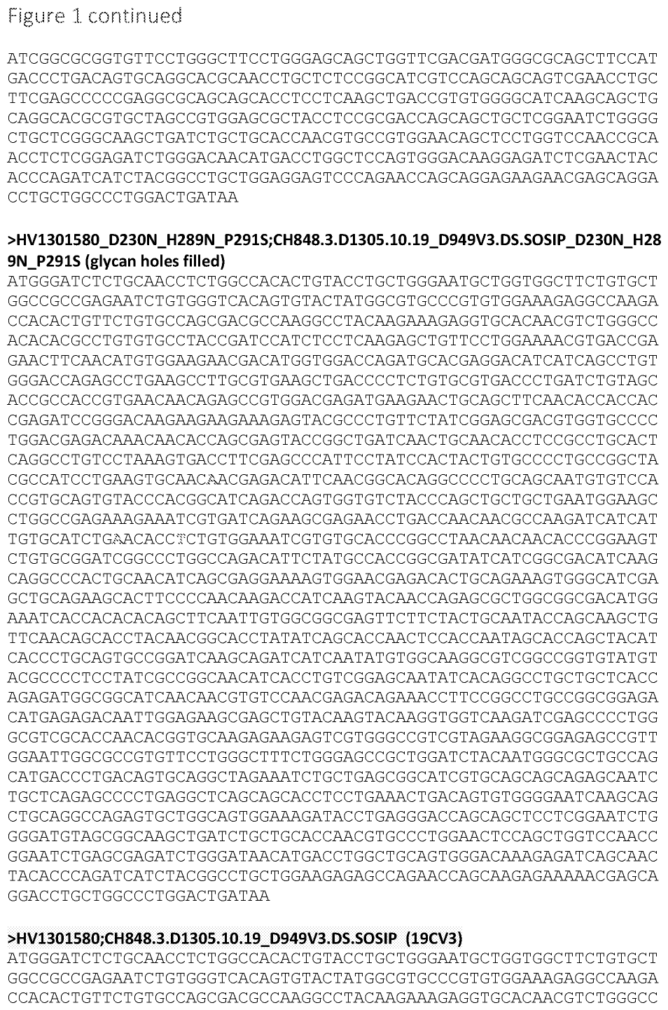

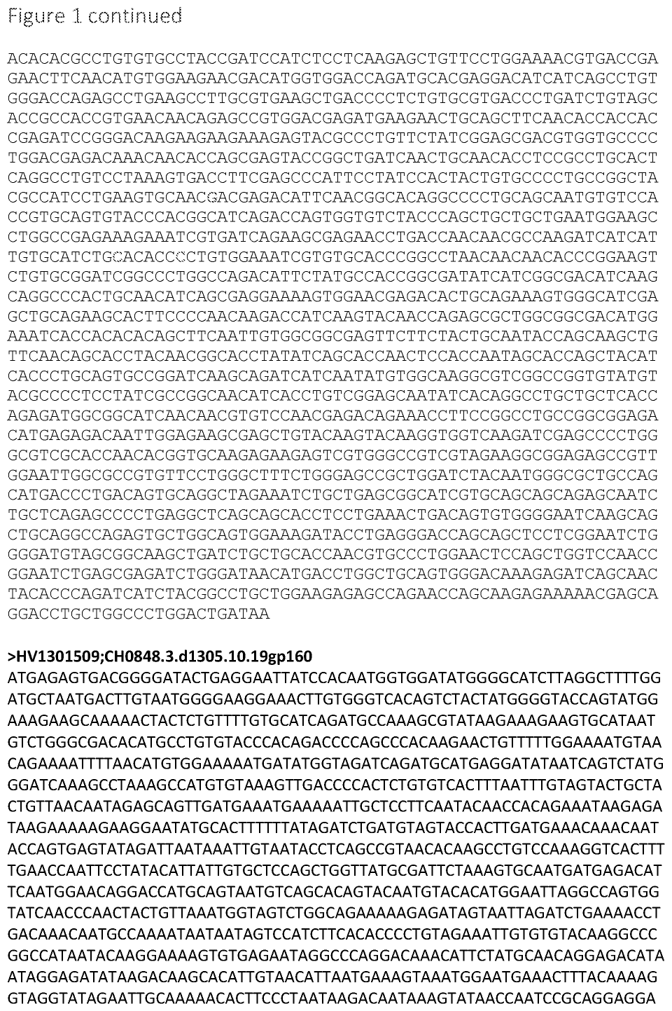

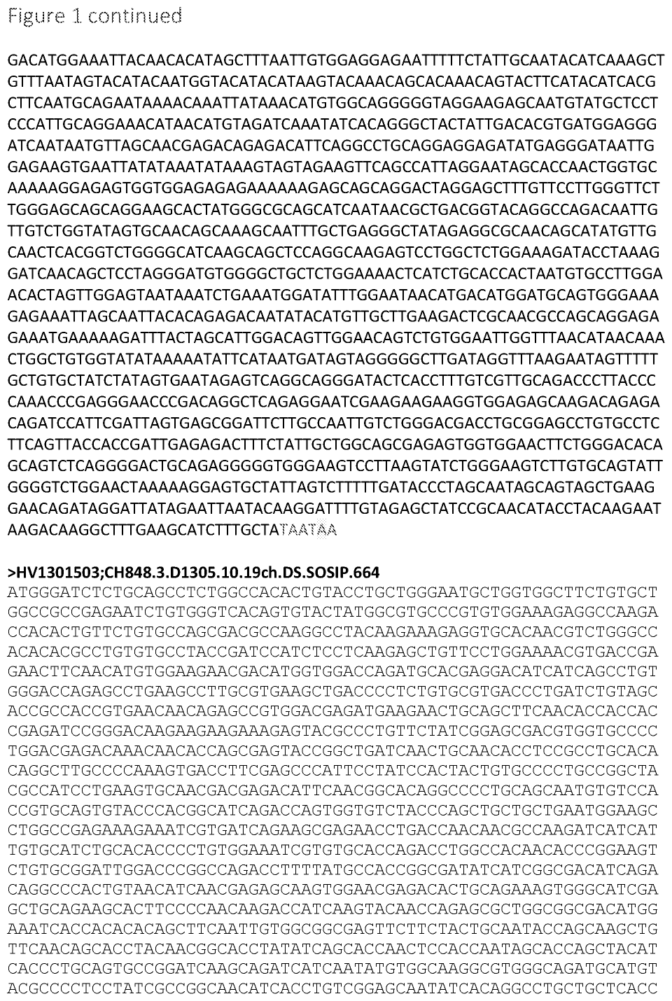

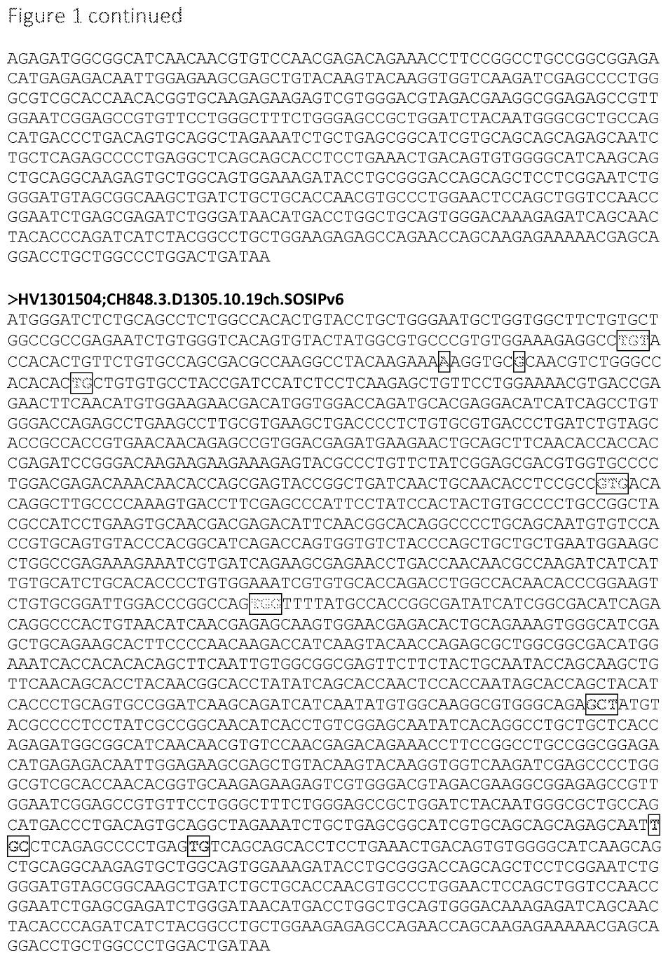

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR.” See Chakrabarti et al. Journal of Virology vol. 76, pp. 5357-5368 (2002) see for example FIG. 1, and second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleavage efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. April; 87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR is changed to ERVVEREKE, and is one example of an uncleaved gp140 form. Another example is the gp140C form which has the REKR site changed to SEKS. See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) at for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CXX, wherein X can be any amino acid) and “VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVHNVWATHACVPTDPNPQE . . . (rest of envelope sequence is indicated as “ . . . ”). In other embodiments, the delta N-design described for CH505 T/F envelope can be used to make delta N-designs of other envelopes. In certain embodiments, the invention relates generally to an HIV-1 envelope immunogen, gp160, gp120, or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the invention provides composition and methods which use a selection of Envs, as gp120s, gp140s cleaved and uncleaved, gp145s, gp150s and gp160s, stabilized and/or multimerized trimers, as proteins, DNAs, RNAs, or any combination thereof, administered as primes and boosts to elicit immune response. Envs as proteins could be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technology, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M. smegmatis) (Yu, J S et al. Clinical Vaccine Immunol. 14: 886-093,2007; ibid 13: 1204-11,2006), and recombinant vaccinia type of vectors (Santra S. Nature Med. 16: 324-8, 2010), for example but not limited to ALVAC, replicating (Kibler K V et al., PLoS One 6: e25674, 2011 Nov. 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See for example technologies developed by incellart.

mRNA

In some embodiments the antigens are nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, each content is incorporated by reference in its entirety. mRNAs delivered in LNP formulations have advantages over non-LNPs formulations. See US Pub 20180028645A1.

In certain aspects the invention contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence could be used. In some embodiments the leader sequence is the endogenous leader sequence. Most of the gp120 and gp160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA). Most of the chimeric designs include CD5 leader sequence. A skilled artisan appreciates that when used as immunogens, and for example when recombinantly produced, the amino acid sequences of these proteins do not comprise the leader peptide sequences.

The immunogenic envelopes can also be administered as a protein prime and/or boost alone or in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (μg) or milligram of a single immunogenic nucleic acid. Recombinant protein dose can range from a few μg micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to 3M052, alum, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen (Leroux-Roels et al., IABS Conference, April 2013). In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355 Foxo1 Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

Multimeric Envelopes

Presentation of antigens as particulates reduces the B cell receptor affinity necessary for signal transduction and expansion (see Baptista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See e.g. He et al. Nature Communications 7, Article number: 12041 (2016), doi:10.1038/ncomms12041; Bamrungsap et al. Nanomedicine, 2012, 7 (8), 1253-1271.

To improve the interaction between the naïve B cell receptor and immunogens, envelope designed can be created to wherein the envelope is presented on particles, e.g. but not limited to nanoparticle. In some embodiments, the HIV-1 Envelope trimer could be fused to ferritin. Ferritin protein self assembles into a small nanoparticle with three fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology 2015 12:82, DOI: 10.1186/s12977-015-0210-4.

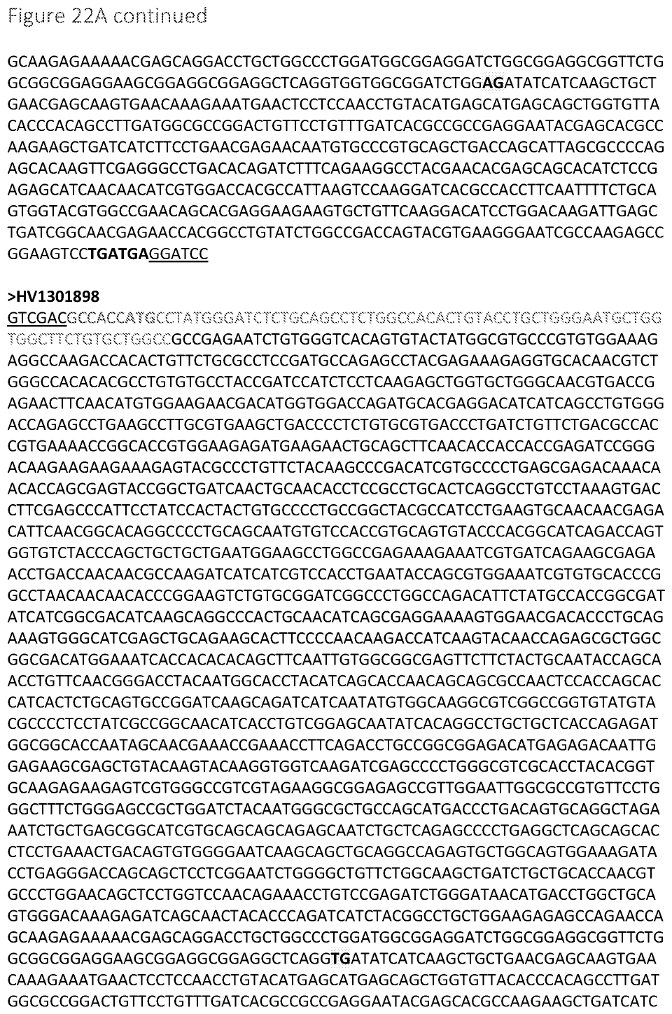

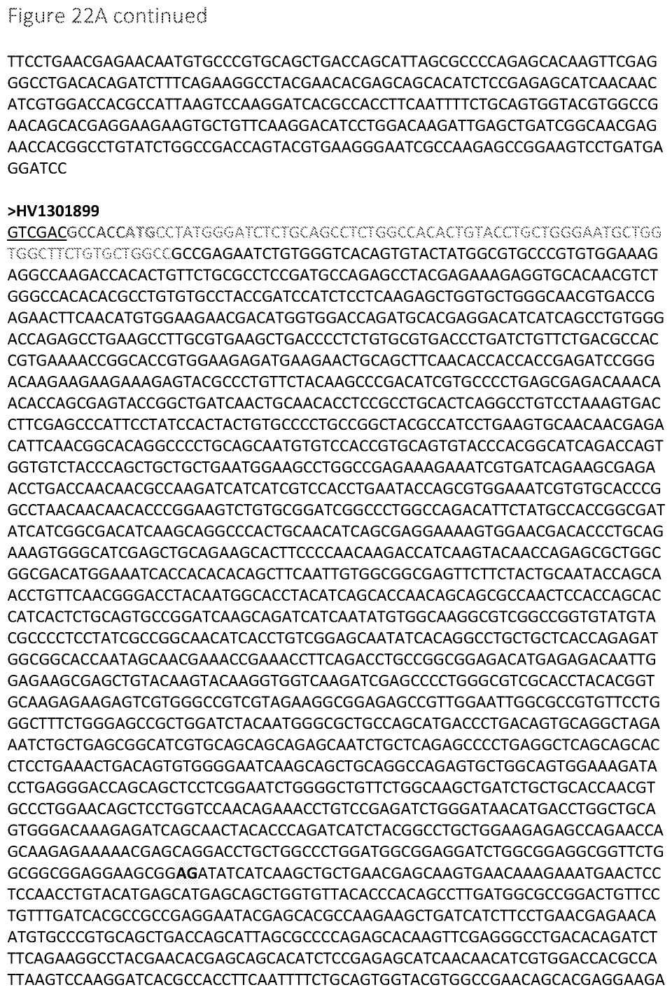

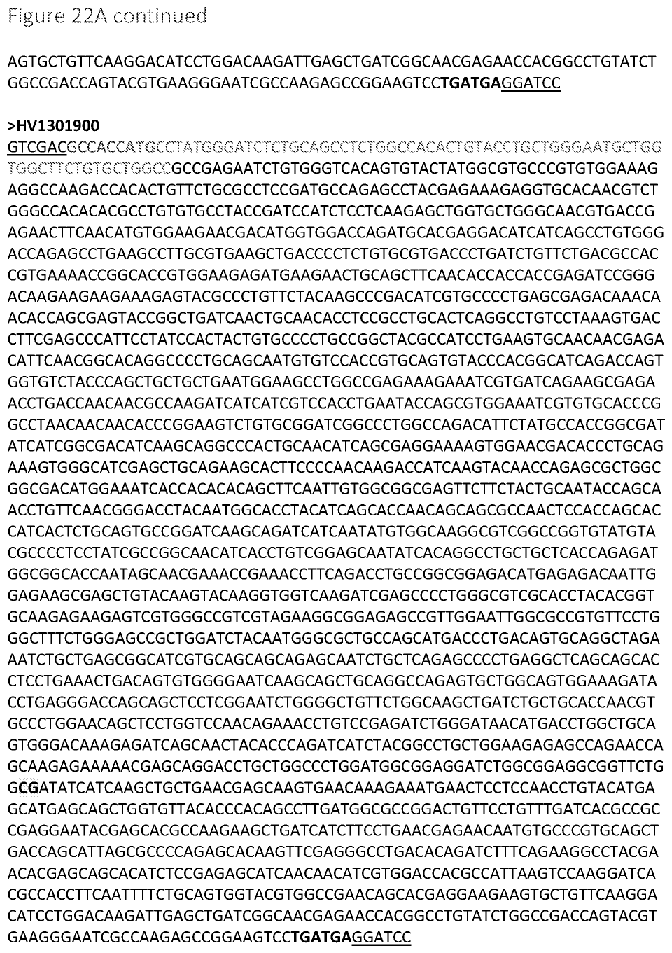

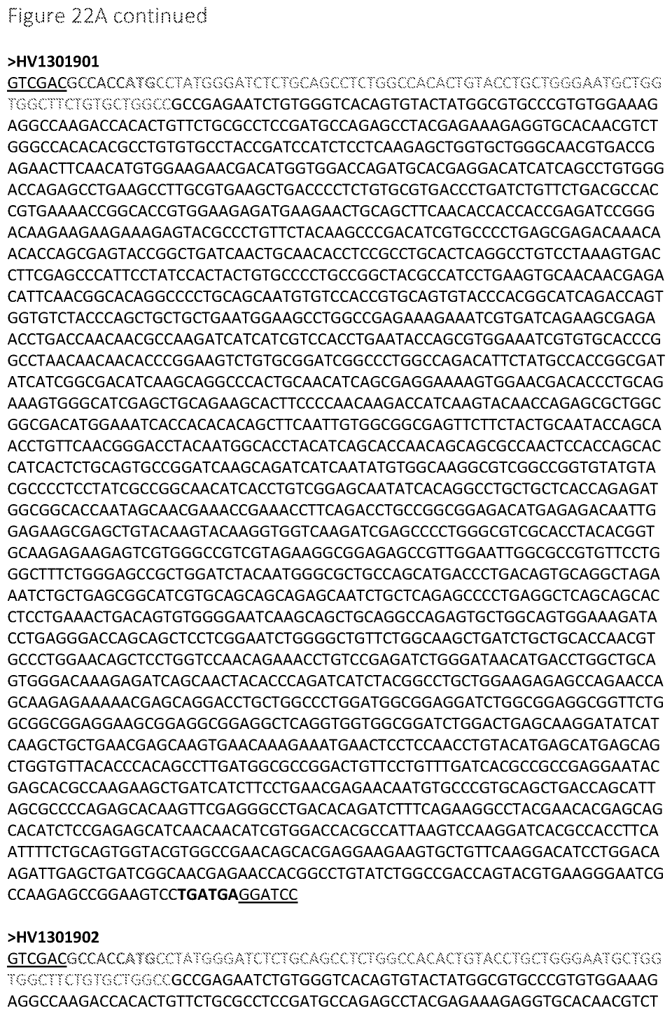

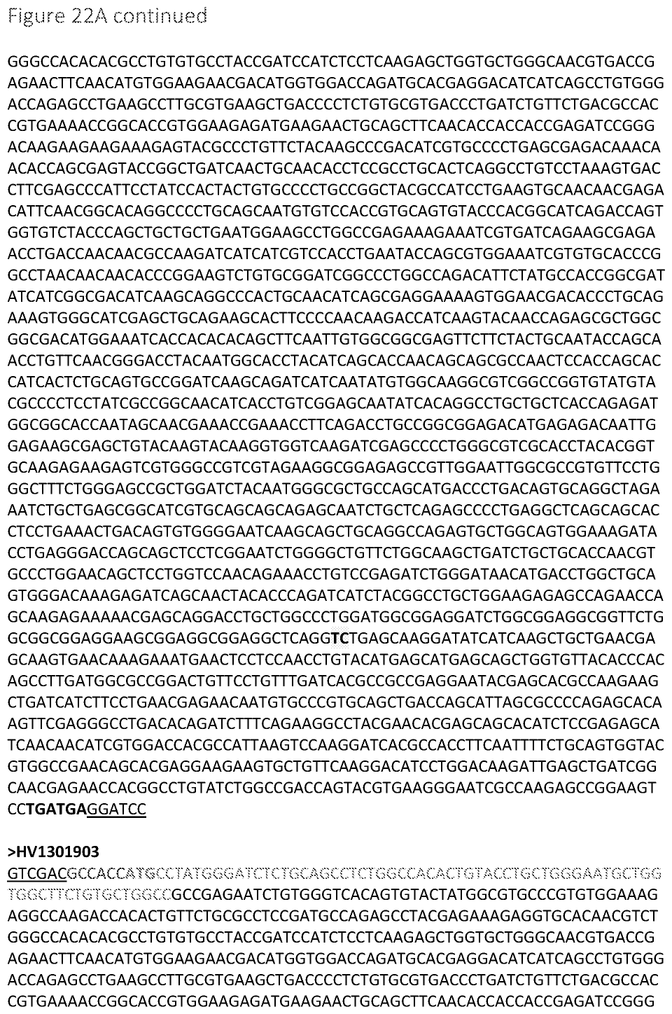

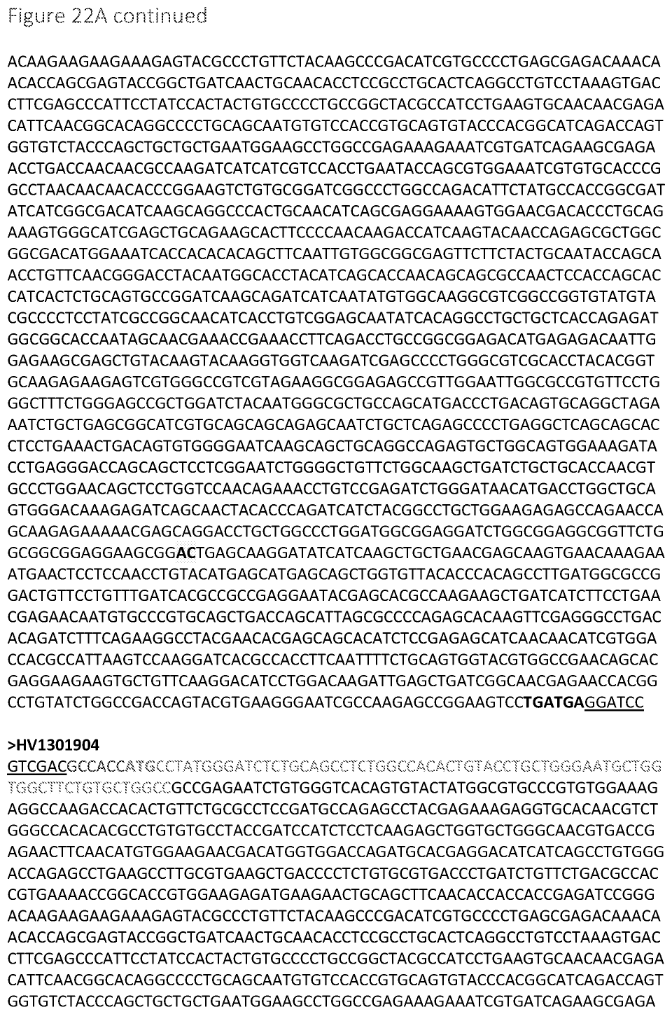

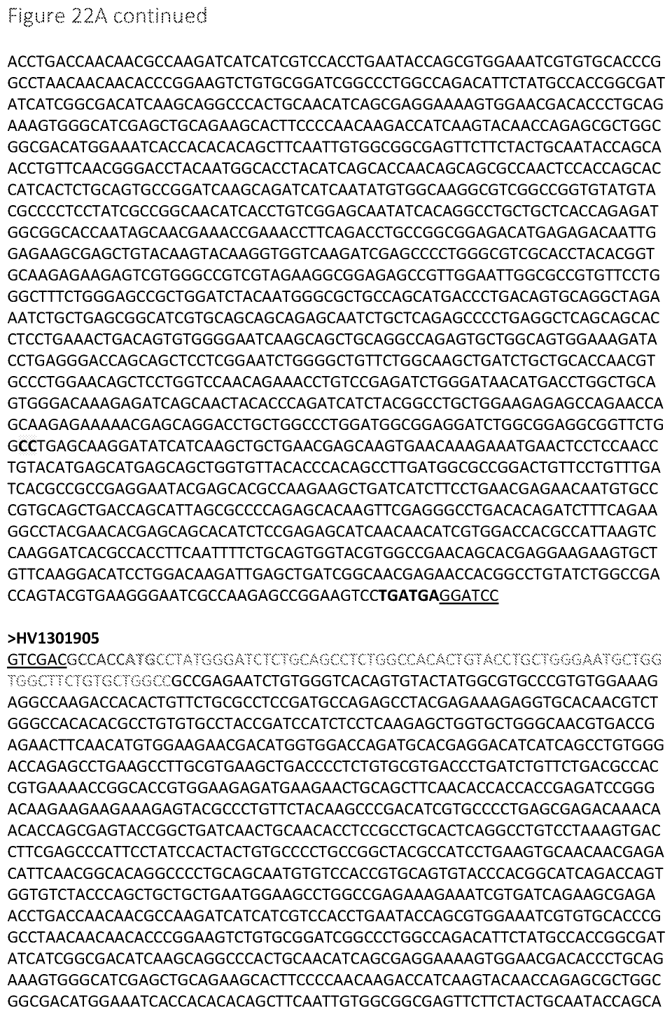

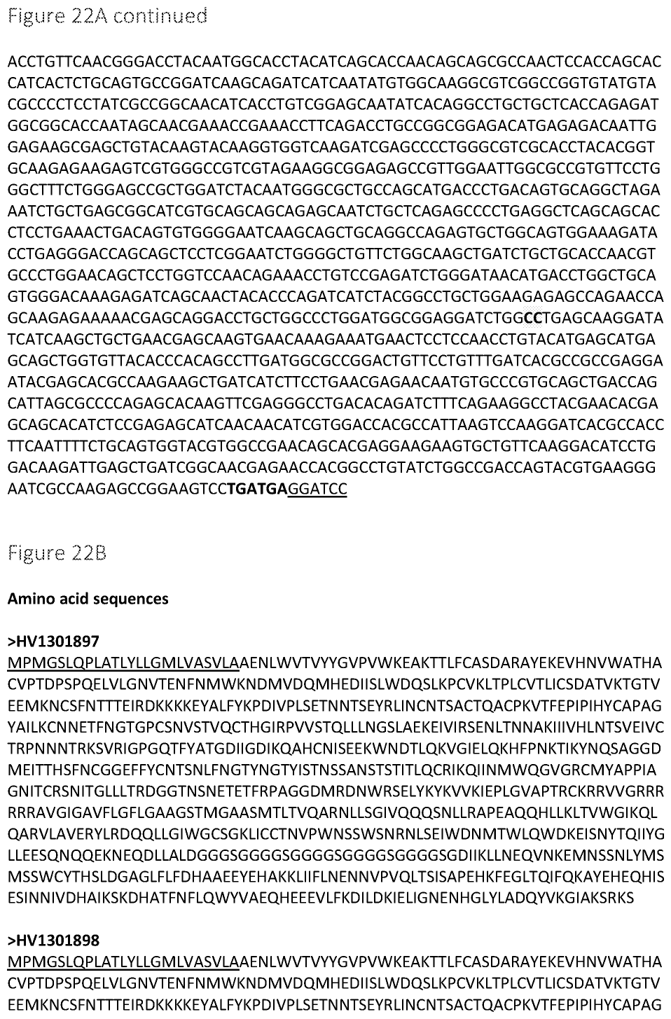

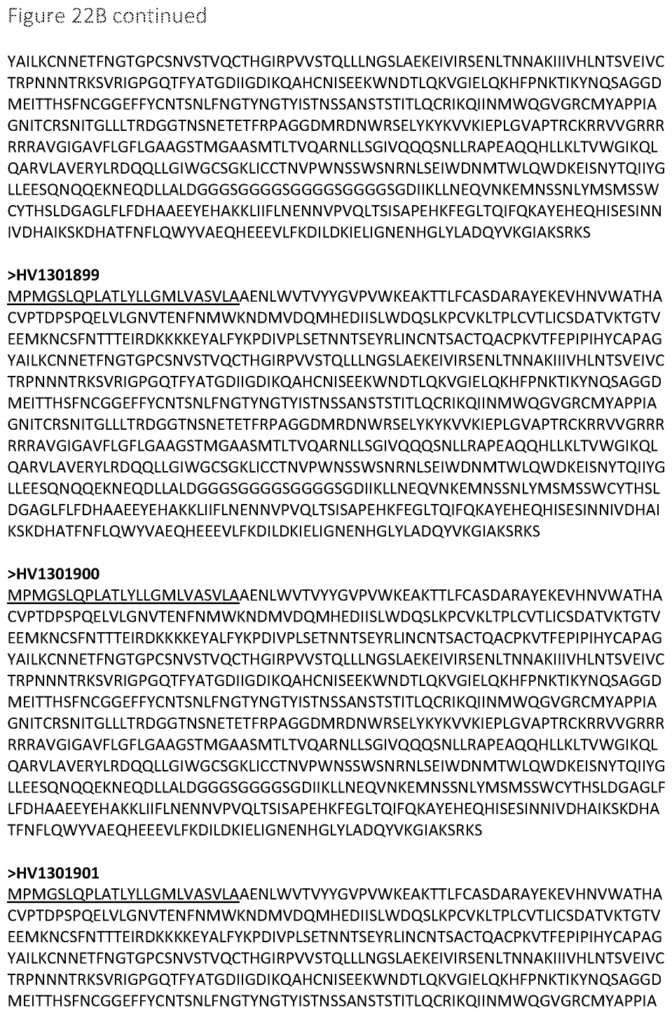

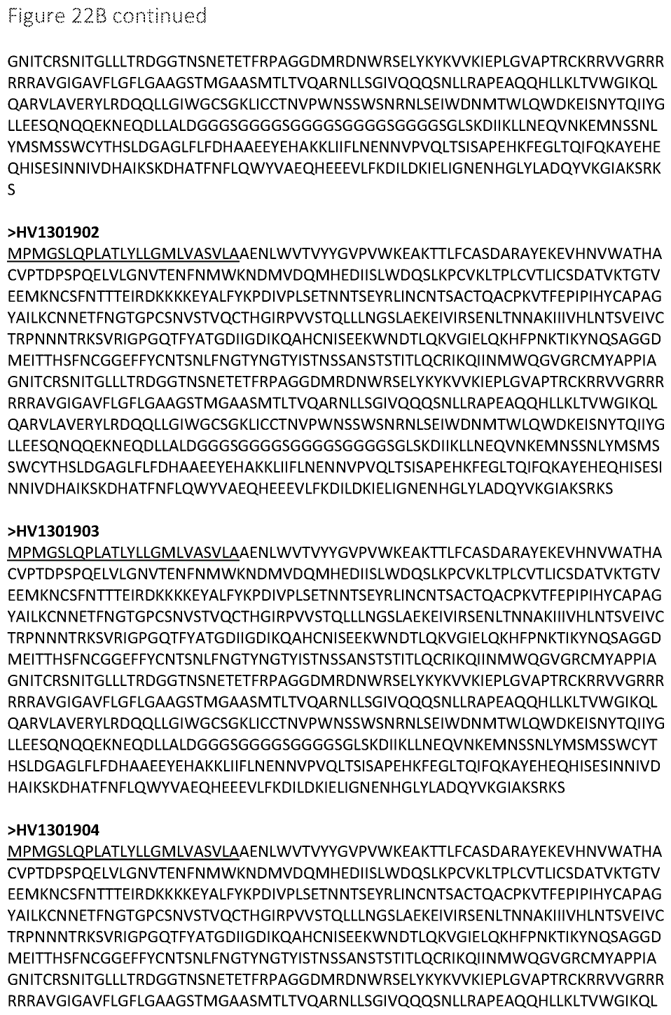

Ferritin nanoparticle linkers: The ability to form HIV-1 envelope ferritin nanoparticles relies self-assembly of 24 ferritin subunits into a single ferritin nanoparticle. The addition of a ferritin subunit to the c-terminus of HIV-1 envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically hindering the association of ferritin subunits. Thus, ferritin can be designed with elongated glycine-serine linkers to further distance the envelope from the ferritin subunit. To make sure that the glycine linker is attached to ferritin at the correct position, constructs can be created that attach at second amino acid position or the fifth amino acid position. The first four n-terminal amino acids of natural Helicobacter pylori ferritin are not needed for nanoparticle formation but may be critical for proper folding and oligomerization when appended to envelope. Thus, constructs can be designed with and without the leucine, serine, and lysine amino acids following the glycine-serine linker. The goal will be to find a linker length that is suitable for formation of envelope nanoparticles when ferritin is appended to most envelopes. For non-limiting embodiments, linker designs see FIGS. 22A-B.

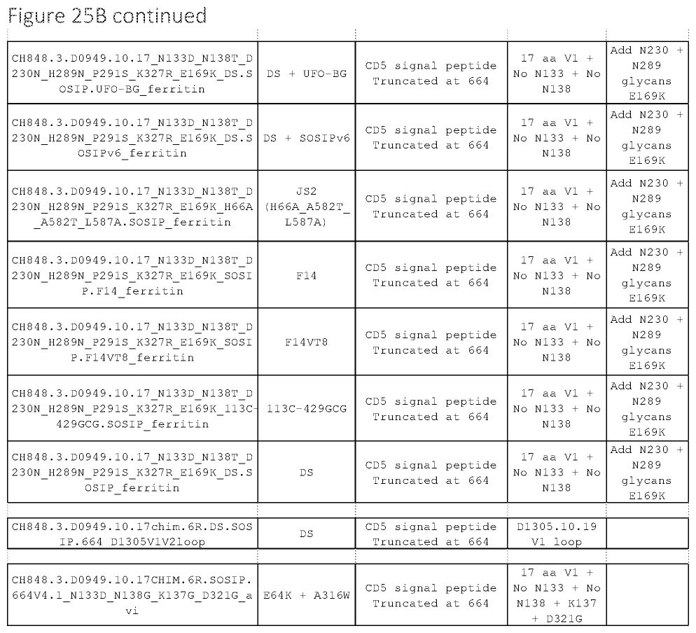

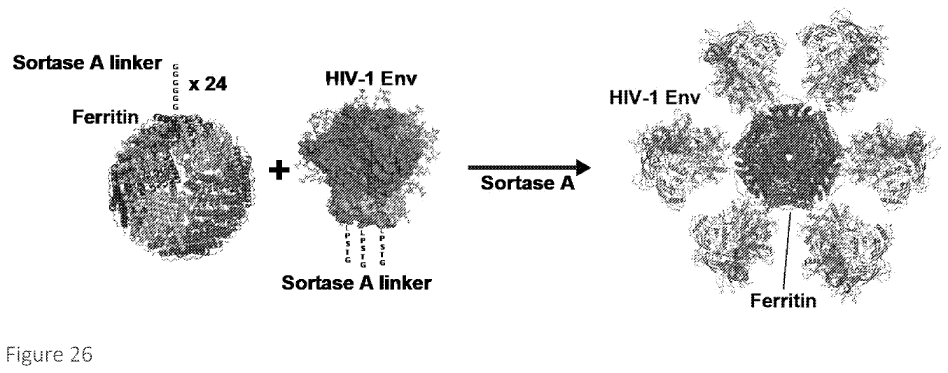

Another approach to multimerize expression constructs uses staphylococcus sortase A transpeptidase ligation to conjugate inventive envelope trimers to cholesterol. The trimers can then be embedded into liposomes via the conjugated cholesterol. To conjugate the trimer to cholesterol either a C-terminal LPXTG tag or a N-terminal pentaglycine repeat tag is added to the envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles. See FIG. 26.

The invention provides design of envelopes and trimer designs wherein the envelope comprises a linker which permits addition of a lipid, such as but not limited to cholesterol, via a sortase A reaction. See e.g. Tsukiji, S. and Nagamune, T. (2009), Sortase-Mediated Ligation: A Gift from Gram-Positive Bacteria to Protein Engineering. ChemBioChem, 10: 787-798. doi:10.1002/cbic.200800724; Proft, T. Sortase-mediated protein ligation: an emerging biotechnology tool for protein modification and immobilisation. Biotechnol Lett (2010) 32: 1. doi:10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35(8):4411-7; Pritz et al. J. Org. Chem. 2007, 72, 3909-3912.

The lipid modified envelopes and trimers could be formulated as liposomes. Any suitable liposome composition is contemplated.

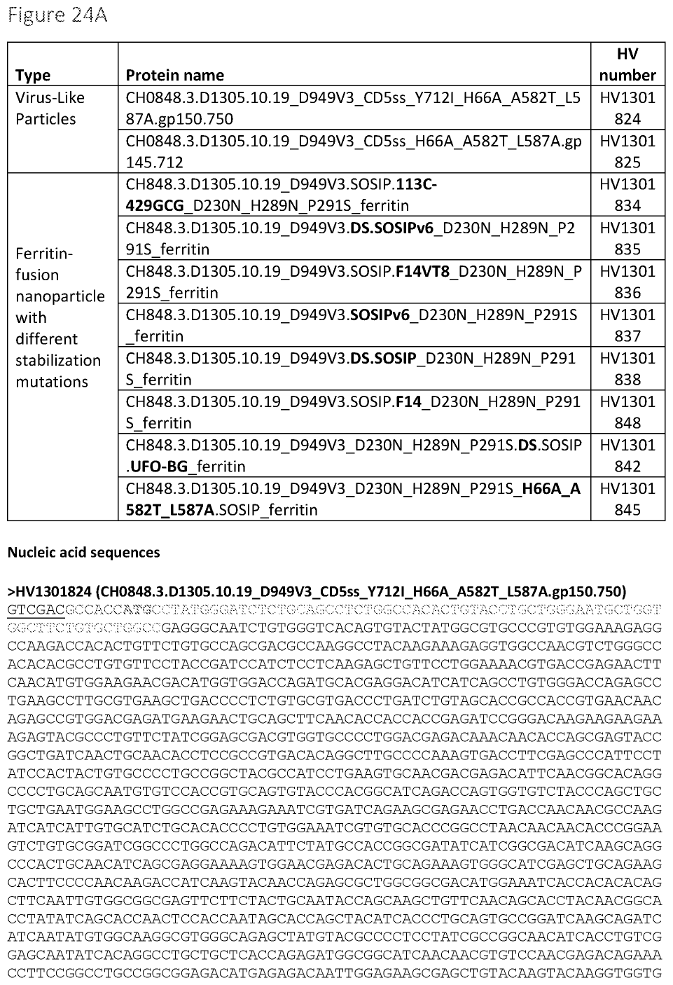

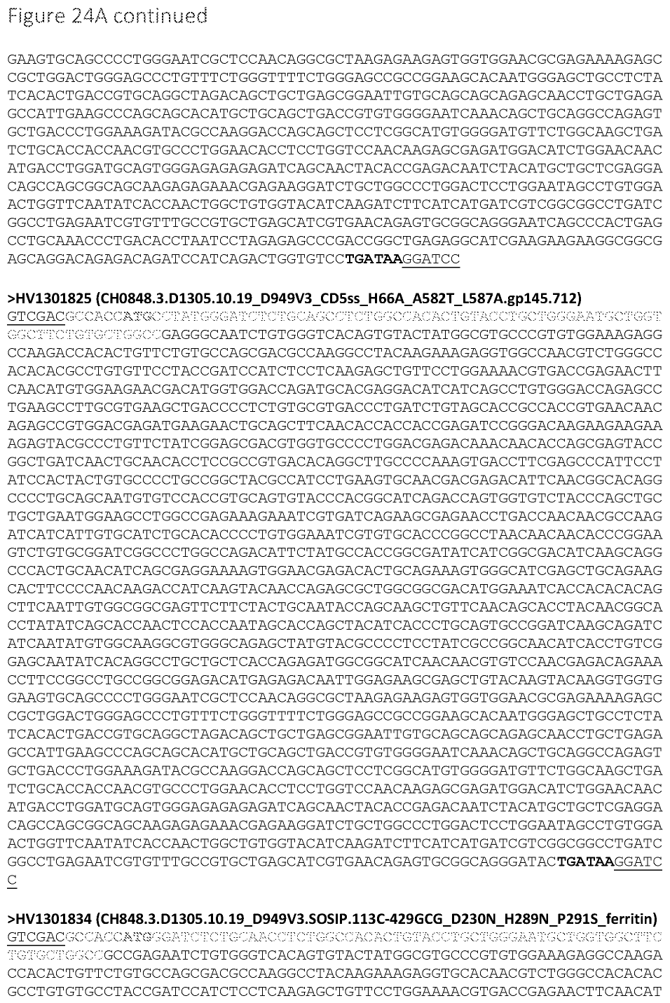

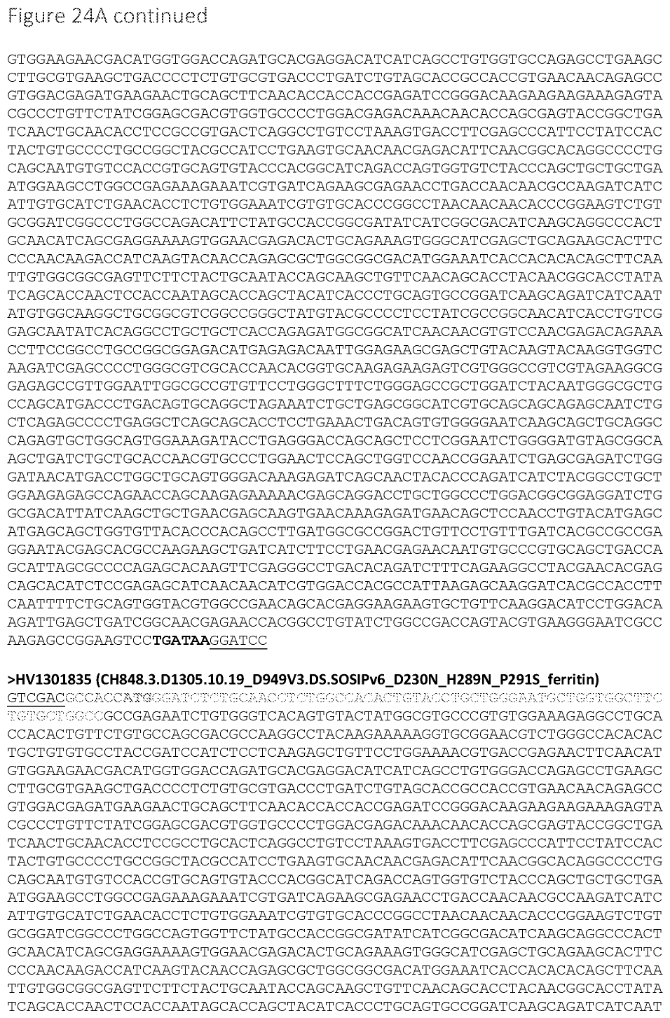

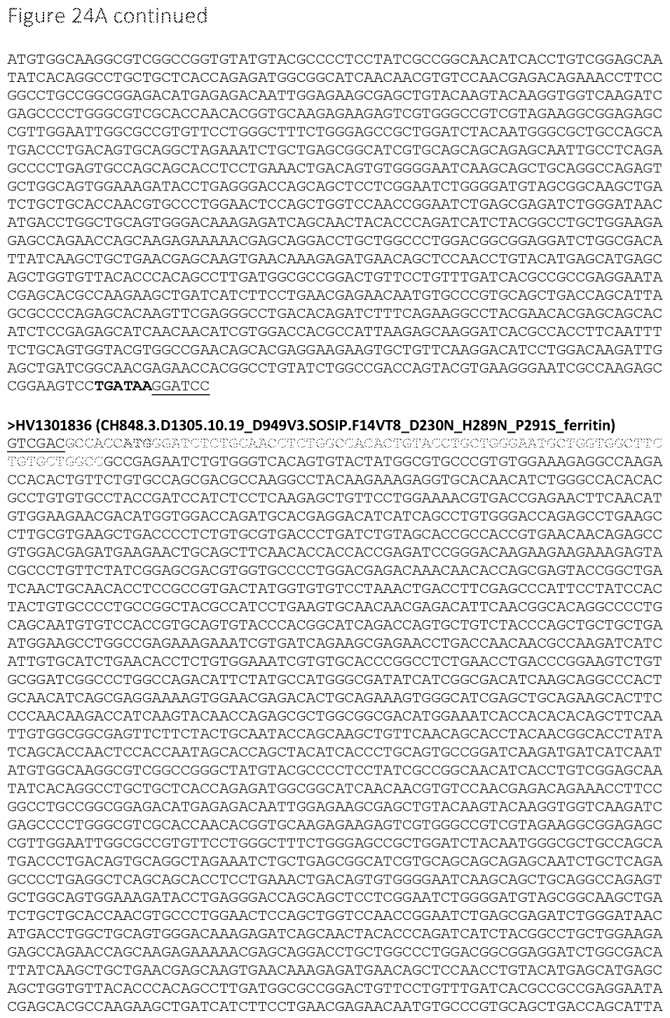

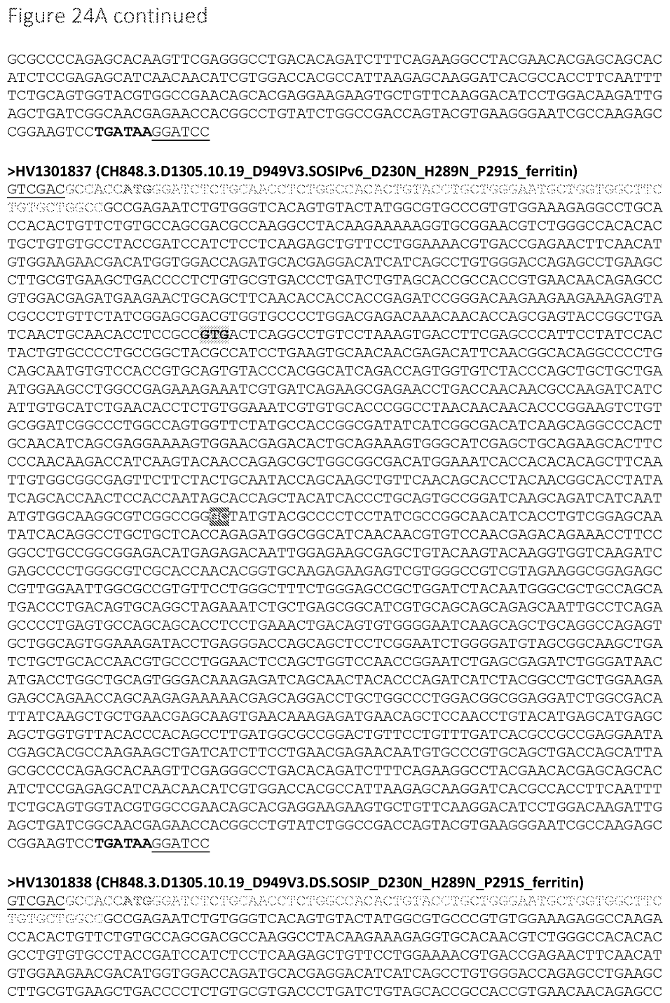

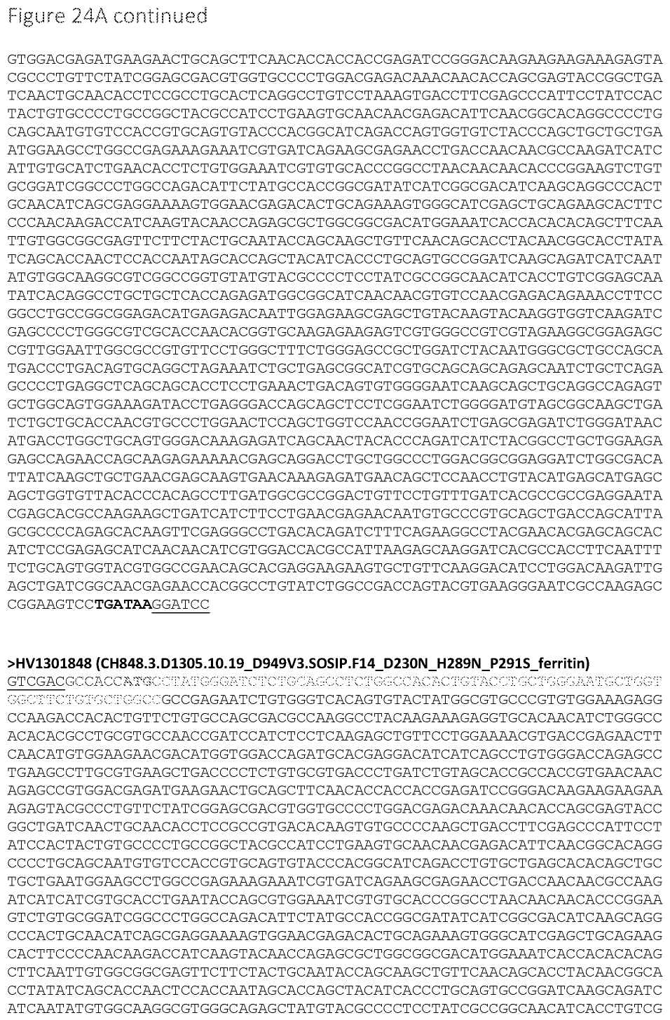

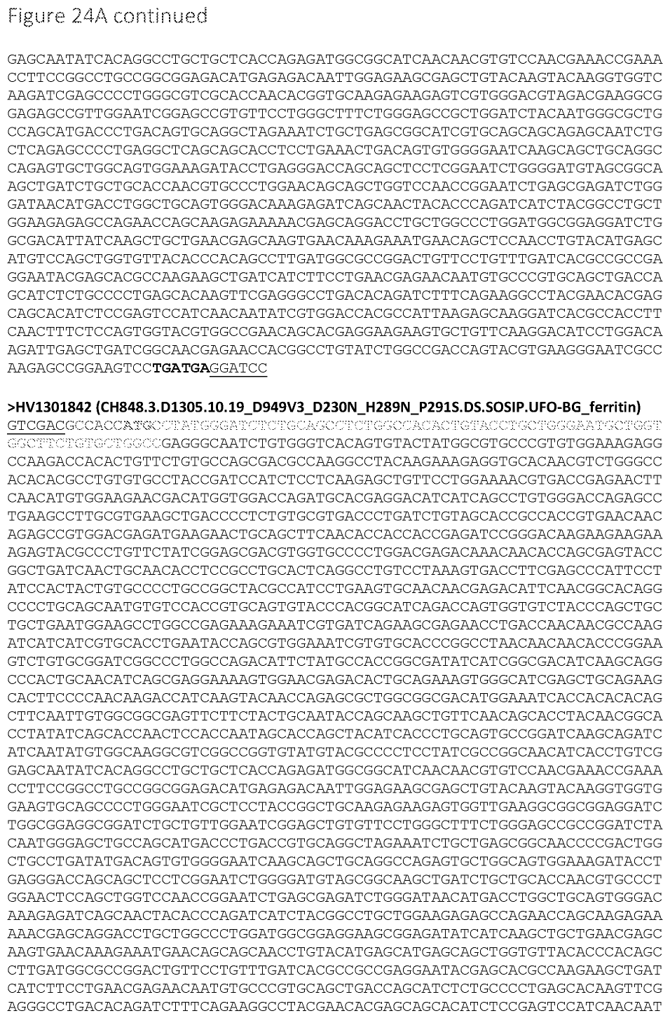

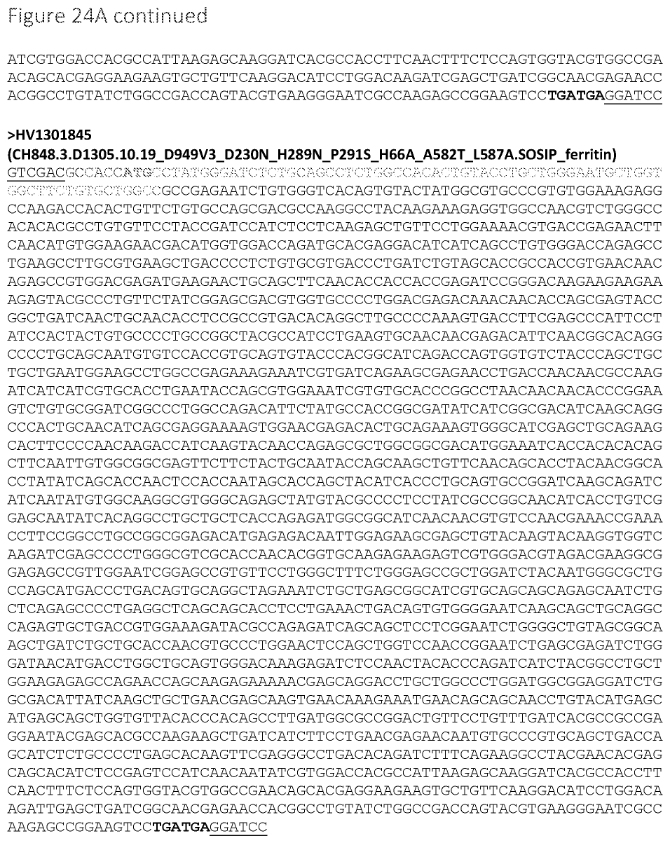

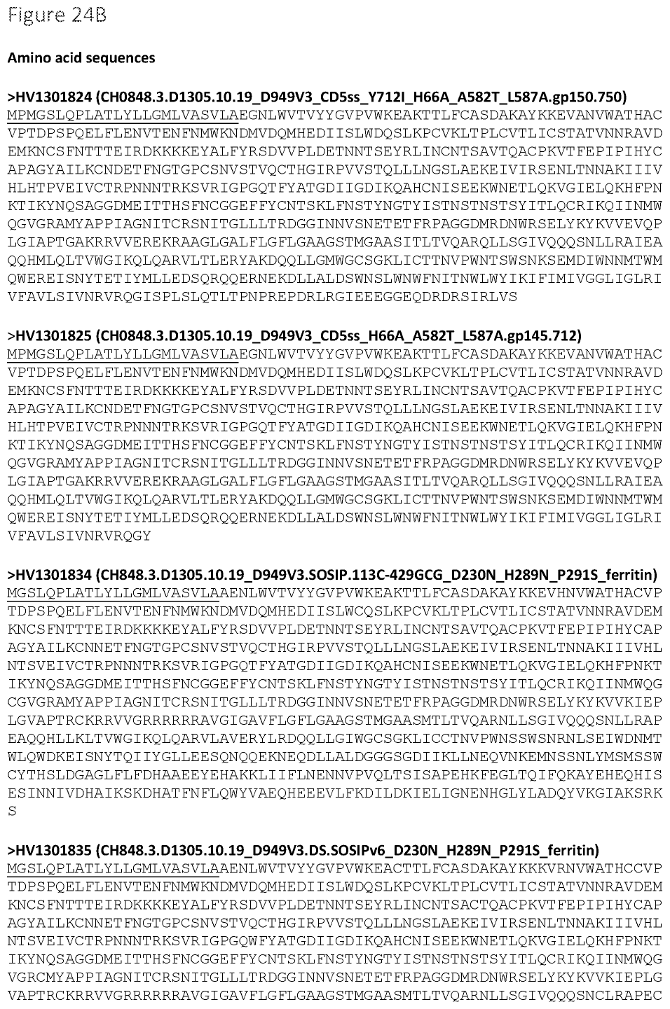

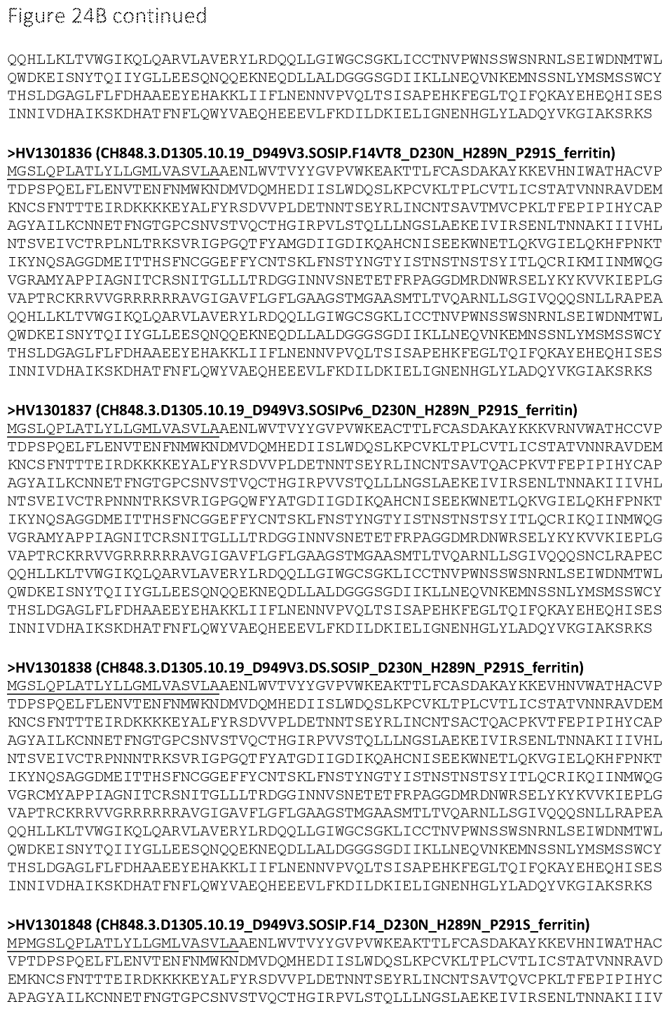

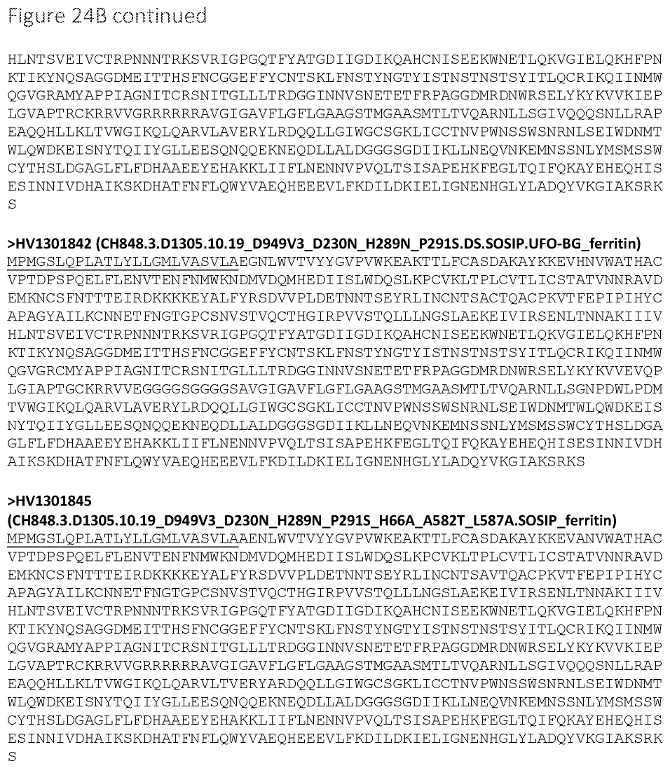

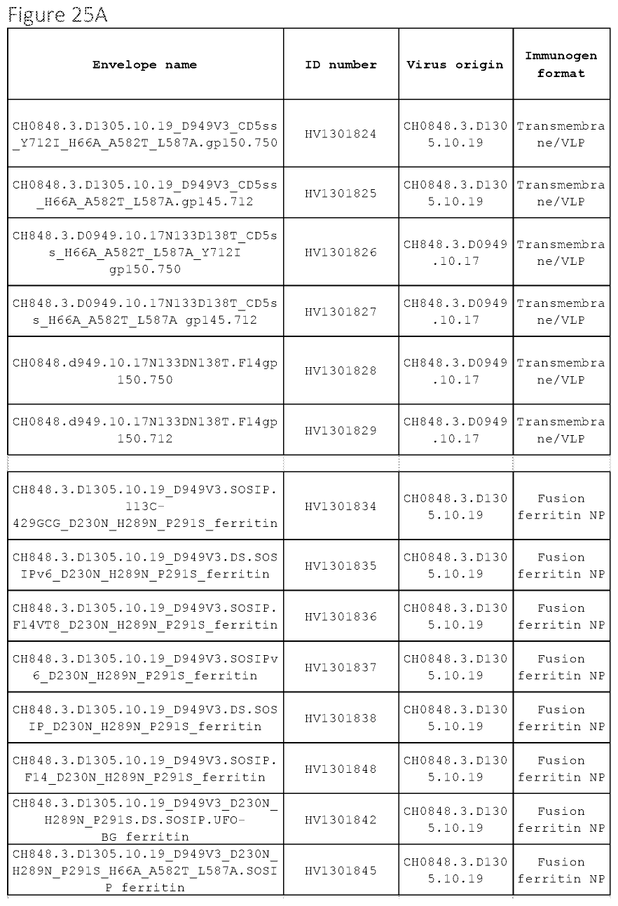

Non-limiting embodiments of envelope designs for use in sortase A reaction are shown in FIG. 24 B-D of WO2017/151801, incorporated by reference in its entirety.

Additional sortase linkers could be used so long as their position allows multimerization of the envelopes.

Table 1 shows a summary of sequences described herein.

| | Amino acid, | | |
|---|---|---|---|
| Name | nucleic acid | design | FIG./Note |
| HV1301580_D230N_H289N_P291S; | Nt | | 1 |
| CH848.3.D1305.10.19_D949V3.DS.SOSIP_D230N_H289N_P291S | aa | | 2 |
| (glycan hole filled ) | | | |
| >HV1301502_D1305V1; | Nt | | 1 |
| JRFL_SOSIPv6_V1_PNGS_D1305V1 (V1 loop from 10.19) | aa | | 2 |
| >HV1301405_D1305V1; | Nt | | 1 |
| CON-Schim.6R.DS.SOSIP.664_OPT_D1305V1 (V1 loop from 10.19 | aa | | 2 |
| isolate) | | | |
| >HV1301580_D230N_H289N_P291S; | Nt | | 1 |
| CH848.3.D1305.10.19_D949V3.DS.SOSIP_D230N_H289N_P291S | aa | | 2 |
| (glycan holes filled) | | | |
| >HV1301580; | Nt | 19CV3 | 1 |
| CH848.3.D1305.10.19_D949V3.DS.SOSIP (19CV3) | aa | | 2 |
| >HV1301509; | Nt | | 1 |
| CH0848.3.d1305.10.19gp160 | aa | | 2 |
| >HV1301503; | Nt | | 1 |
| CH848.3.D1305.10.19ch.DS.SOSIP.664 | aa | | 2 |
| >HV1301504; | Nt | | 1 |
| CH848.3.D1305.10.19ch.SOSIPv6 | aa | | 2 |
| >HV1301580_C_SORTA; | Aa | | 3 |
| CH848.3.D1305.10.19_D949V3.DS.SOSIP_C_SORTA | nt | | 3 |

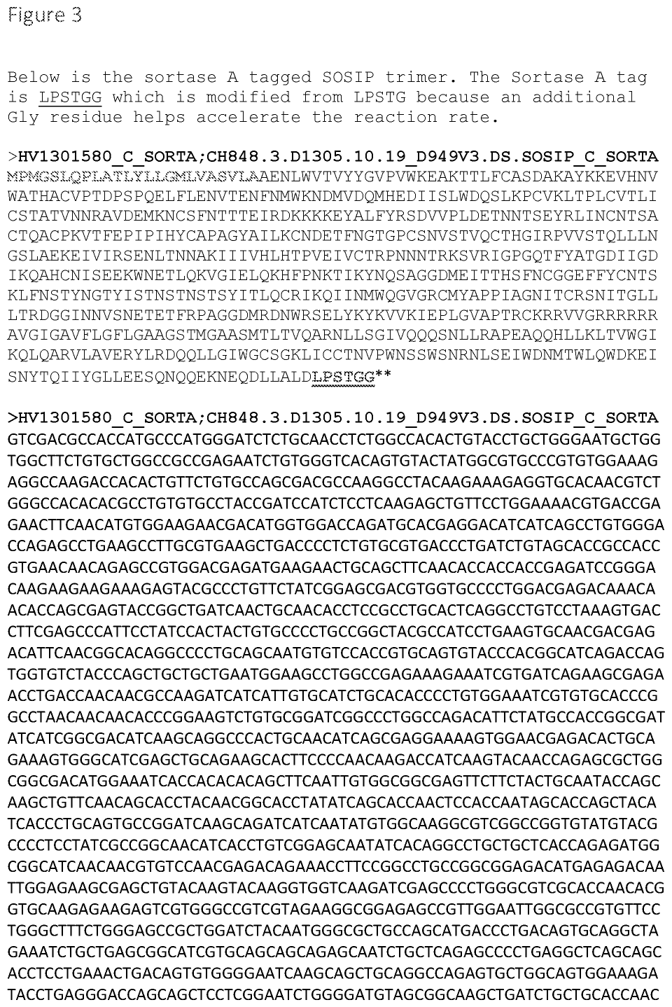

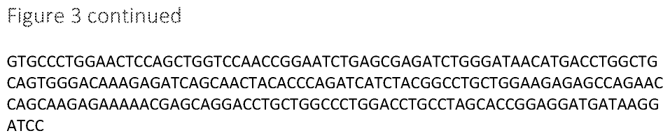

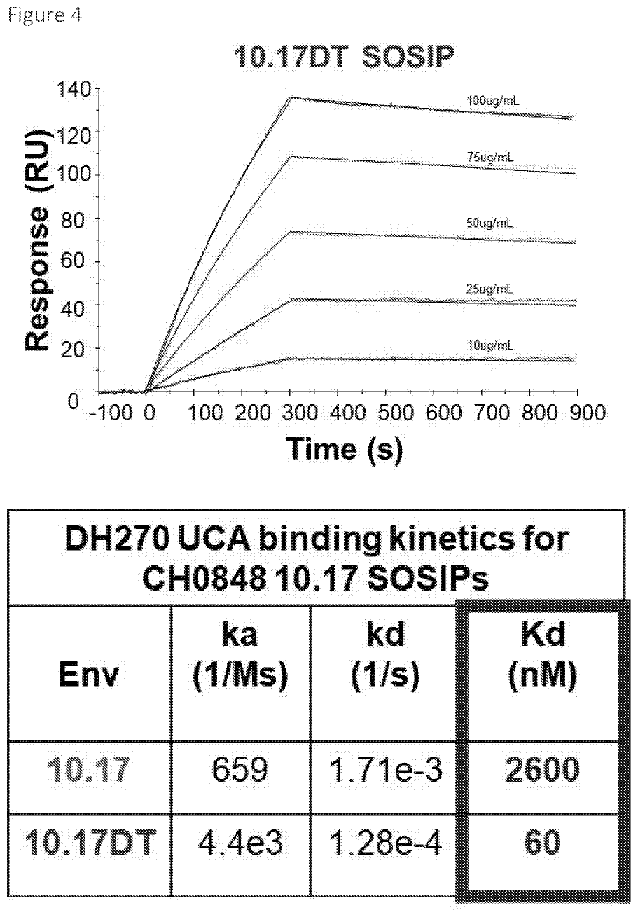

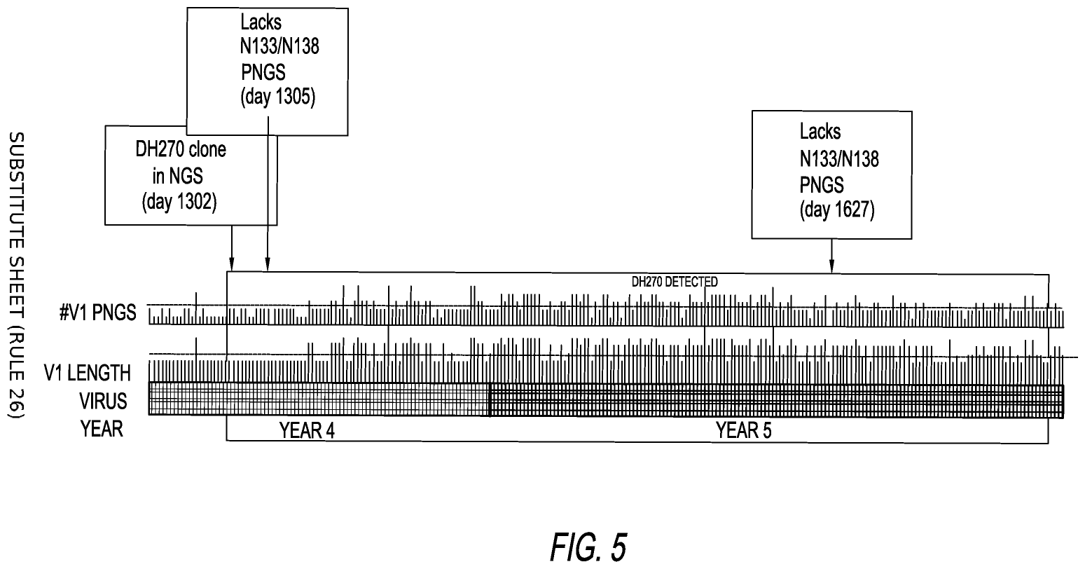

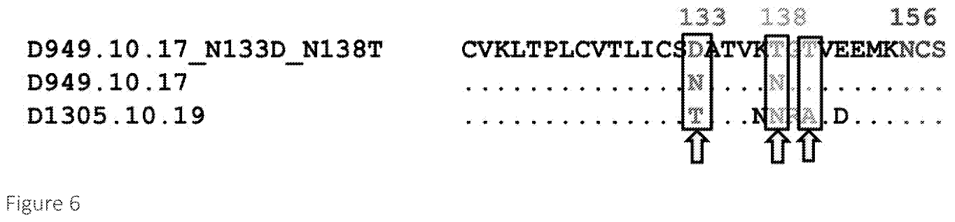

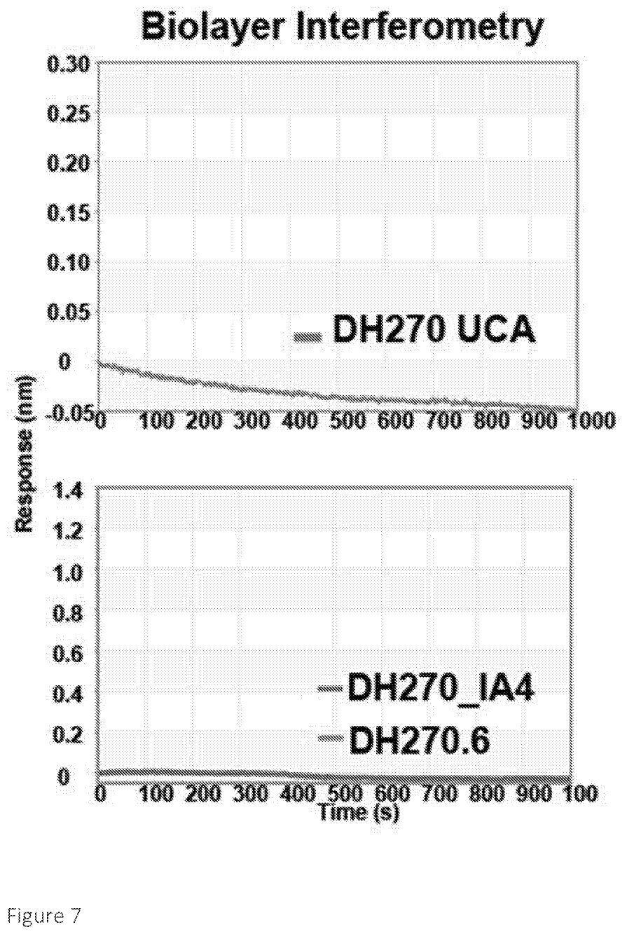

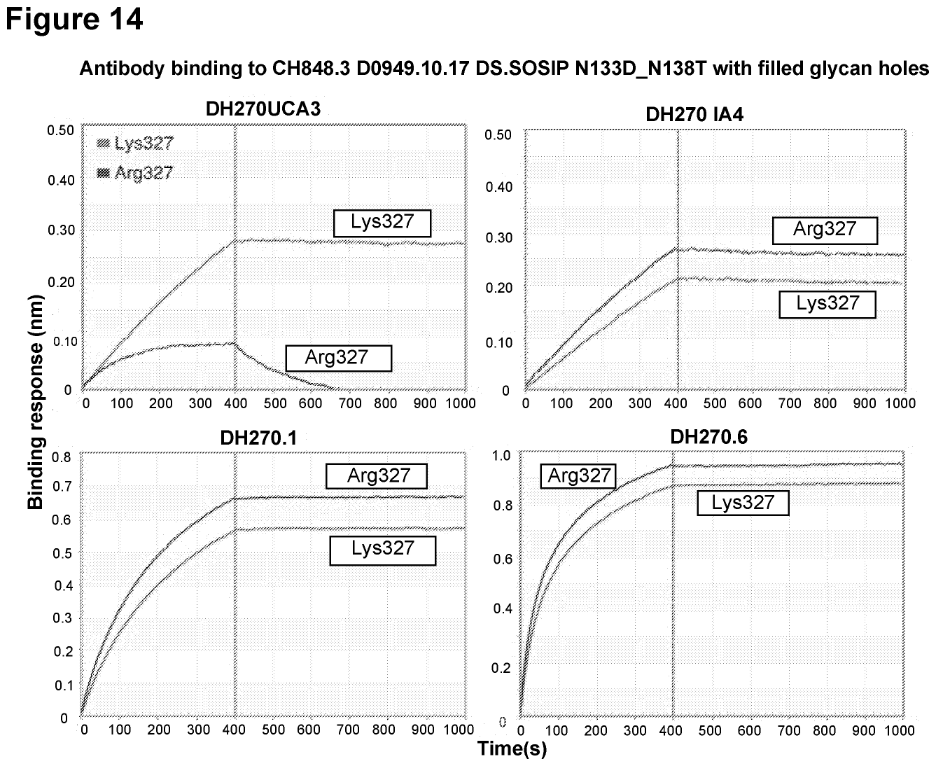

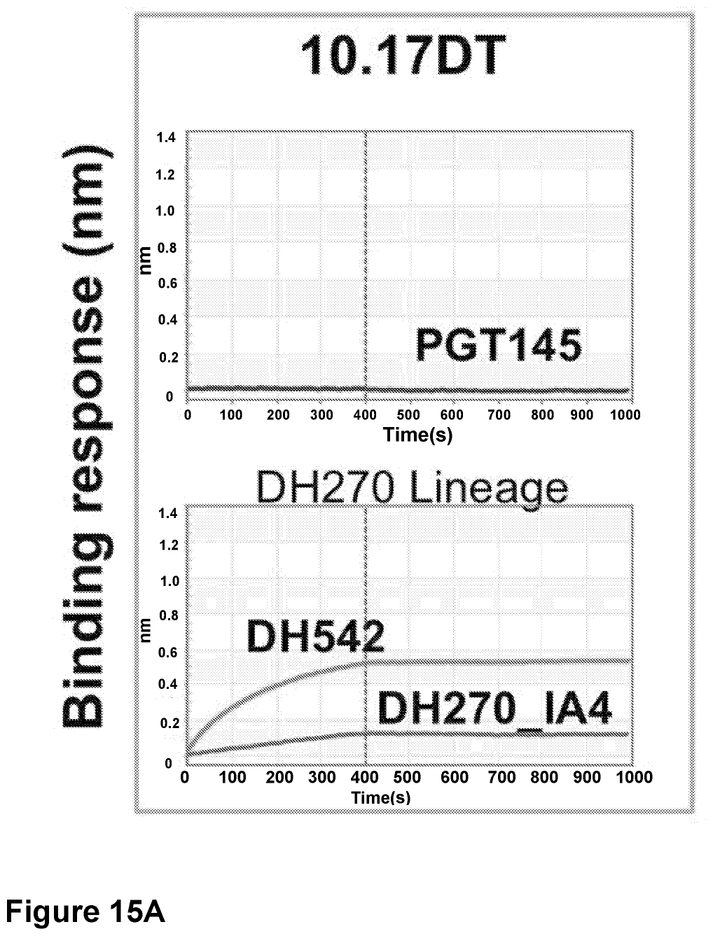

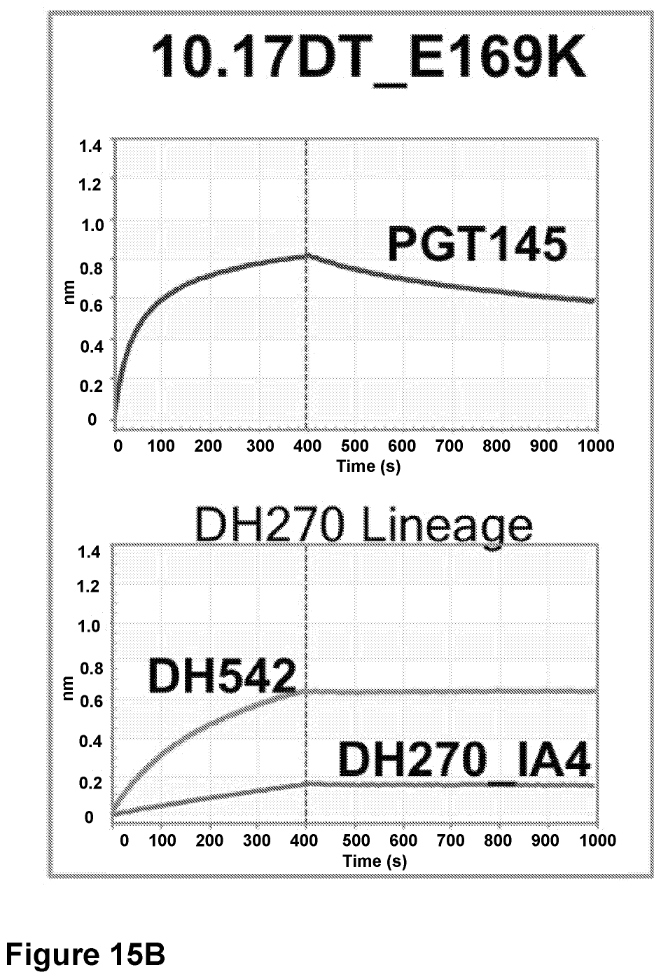

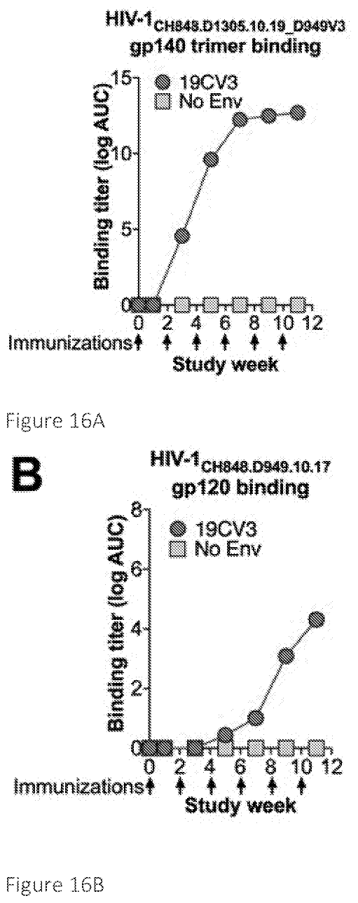

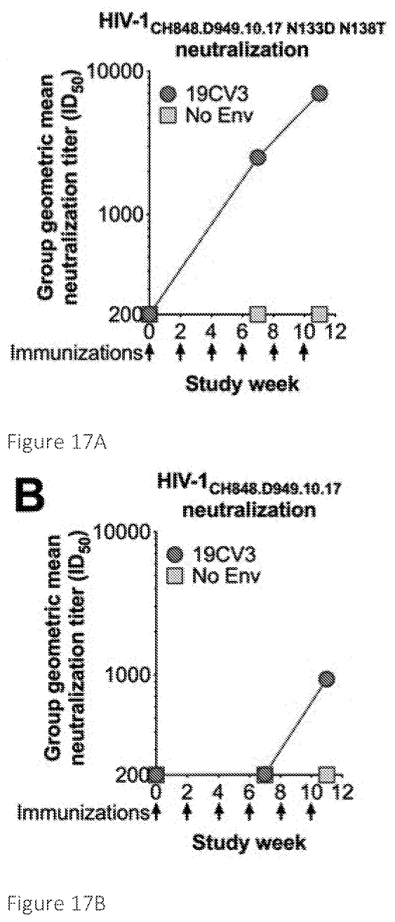

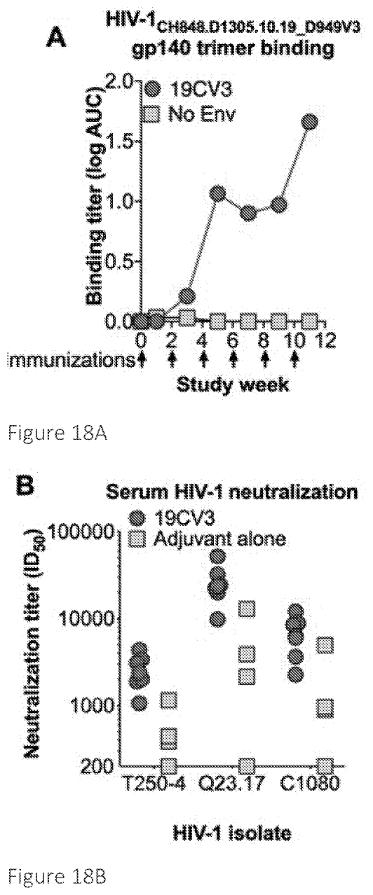

Table 2 shows a summary of modifications to envelopes described herein

| | FIG./SEQ ID | | V3 glycosylation | UCA and other |
|---|---|---|---|---|
| Envelope | No | V1 region | sites | Ab binding |
| 10.17 | DU4918 | 17aa | N301 and N332 | |
| 10.17 DT | DU4918 | 17aa N133D | N301 and N332 | DH270UCA |
| | | N138T | | |
| | | effectively lacks | | |
| | | glycosylation | | |
| | | sites | | |
| 10.19 | FIG. 1 | 17aa V1 region | No glycosylation | CH01 UCA |
| | | lacks N133 and | sites at N295, | |
| | | N138 | N301, N332 | |
| | | glycosylation | | |
| | | sites | | |
| 10.19 plus | FIG. 1, FIG. 3 | 17aa V1 region | Add V3 regions | CH01 UCA |
| V3 loop of | | lacks N133 and | from 10.17 has | DH270UCA |
| 10.17 | | N138 | five aa difference | VRC26 UCA |
| (19CV3) | | glycosylation | from 10.19 | |
| | | sites | | |
| 10.19 env | FIG. 14 | | At least changes | |
| based with | | | #2, 4, 5, and/or | |
| fewer than | | | “GDIR” sequence | |
| five aa | | | | |
| changes | | | | |
| compared | | | | |
| to 19CV3; | | | | |
| “GDIR/K” | FIG. 21 | | | |
| Ferritin | FIG. 22 | | | |
| Linker | | | | |
| E169K | FIG. 21 | | | |
| Glycan | FIG. 21, 22, 24 | | | |
| whole filled | | | | |

DH270 light chain binds to N301 glycan. In some embodiments, a N301 gly site is used (e.g. change #2 in row 5 of Table 2, supra).
DH270 heavy chain binds to N332 glycan. In some embodiments, a N332 gly site is used (e.g. changes #4 and #5 in row 5 of Table 2, supra).

V3 glycan Abs bind GDIR. In some embodiments, a change #3 to “GDIR” is needed (e.g. “GDIR” sequence in row 5 of Table 2, supra).

GDIR/K motif: V3-glycan broadly neutralizing antibodies typically contact the c-terminal end of the third variable region on HIV-1 envelope. There are four amino acids, Gly324, Asp325, Ile326, and Arg327, bound by V3-glycan neutralizing antibodies. While Arg327 is highly conserved among HIV-1 isolates, Lys327 also occurs at this site. The CH848.3.D0949.10.17 isolate naturally encodes the less common Lys327. In contrast to CH848.3.D0949.10.17 with the Lys327, the precursor antibody of the DH270 V3-glycan broadly neutralizing antibody lineage barely binds to CH848.3.D0949.10.17 encoding Arg327. Thus, Arg327 is critical for the precursor to bind and the lineage of neutralizing antibodies to begin maturation. However, somatically mutating antibodies on the path to developing neutralization breadth bind better to Env encoding Arg327. See FIG. 14. Thus, Env must encode Lys327 to initiate DH270 lineage development. However, to best interact with affinity maturing DH270 lineage members the Env should encode Arg327. Thus, a plausible vaccine regimen to initiate and select for developing bnAbs would include a priming immunogen encoding, Lys327 and a boosting immunogen encoding Arg327. The Arg327 boosting immunogen would optimally target the affinity maturing DH270 lineage members, while not optimally binding the DH270 antibodies that lack affinity maturation. Non-limiting embodiments of vaccination regimens could include: priming with CH848.3.D0949.10.17 based envelope design also with Lys327, followed by administering of CH848.3.D0949.10.17 based envelope design with Arg327. Non-limiting embodiments of vaccination regimens could include: priming with 19CV3 based envelope design also with Lys327, followed by administering of CH848.3.D0949.10.17 based envelope design with Arg327.

E169K modification: One approach to designing a protective HIV-1 vaccine is to elicit broadly neutralizing antibodies (bnAbs). However, bnAbs against two or more epitopes will likely need to be elicited to prevent HIV-1 escape. Thus, optimal HIV-1 immunogens should be antigenic for multiple bnAbs in order to elicit bnAbs to more than one epitope. The CH848.D949.10.17 HIV-1 isolate was antigenic for V3-glycan antibodies but lacked binding to V1V2-glycan antibodies. Not all viruses from the CH848 individual lacked binding to V1V2-glycan antibodies. For example, the CH848.D1305.10.19 isolate bound well to V1V2-glycan antibody PGT145. We compared the sequence of CH848.D949.10.17 and CH848.D1305.10.19 in the region that is contacted by V1V2-glycan antibodies in crystal structures (McLellan J S, Pancera M, Carrico C, Gorman J, Julien J P, Khayat R, et al. Structure of HIV-1 gp120 V1/V2 domain with broadly neutralizing antibody PG9. Nature. 2011; 480(7377):336-43). Interestingly, the CH848.D949.10.17 and CH848.D1305.10.19 differed in sequence at a known contact site for V1V2-glycan antibodies—position 169 (Doria-Rose N A, Georgiev I, O'Dell S, Chuang G Y, Staupe R P, McLellan J S, et al. A short segment of the HIV-1 gp120 V1N2 region is a major determinant of resistance to V1N2 neutralizing antibodies. J Virol. 2012; 86(15):8319-23). It has been previously shown that mutation of lysine at position 169 eliminates binding to V1V2-glycan antibody PG9 (Doria-Rose N A, Georgiev I, O'Dell S, Chuang G Y, Staupe R P, McLellan J S, et al. A short segment of the HIV-1 gp120 V1N2 region is a major determinant of resistance to V1N2 neutralizing antibodies. J Virol. 2012; 86(15):8319-23). CH848.D1305.10.19 sequence encoded a lysine at position 169 whereas CH848.D949.10.17 sequence encoded a glutamate. Thus, we changed the glutamate (E) to lysine (K) at position 169 of CH848.D949.10.17. This single change in CH848.D949.10.17 enabled V1V2-glycan antibody binding to the envelope. Thus, the E169K adds the V1V2-glycan epitope to the other bnAb epitopes present on CH848.D949.10.17-based envelopes. Overall, the result of the E169K is a CH848.D949.10.17 envelope capable of eliciting more different types of bnAbs.

The invention contemplates any other design, e.g. stabilized trimer, of the sequences described here in. For non-limiting embodiments of additional stabilized trimers see WO2014/042669 (DU4061), WO2017/151801 (DU4716), WO2017/152146 (DU4918) and WO2018/161049 (DU4918), all of which are incorporated by reference in their entirety, and F14 and/or VT8 designs.

F14NT8 designs mutations are listed below (HXB2 numbering) with a brief explanation for each. All were originally placed in BG505 SOSIP. They were then screened via BLI of small scale transfection supernatants. From the BLI data F14, F15 and VT8 were expressed, purified, and screened for CD4 binding and triggering.

These sets of mutations were then put into CH848 10.17 DT and CH505 M5 SOSIP (F14, VT8, and F14+VT8) in addition to a BG505 SOSIP F14+VT8.

Full Set->Pack the BMS-626529 binding site and lock the layers in place

The set of mutations referred to as F1 are V68I, S115V, A204L, V208L, V255W, N377L, M426W, M434W, and H66S.

Elimination* of N377L, M426W, and M434W may avoid over-packing the area. N377 may be important for folding as it is not totally buried. “Elimination” means that an F2 construct includes all F1 mutations except N337L, M426W, and M434W.

The set of mutations referred to as F2 are: V68I, S115V, A204L, V208L, V255W, and H66S

Elimination of S115V may be done if adding a V may be too large for the area where S115 resides.

The set of mutations referred to as F3 are: V68I, A204V, V208L, V255L, and H66S.

Elimination of A204V may be done if adding a V may be too large for the packed region where A204 resides. (Adding E causes opening of the apex.)

The set of mutations referred to as F4 are: V68I, S115V, V208L, V255L, and H66S.

Retention of N377L may be used for the minimal set. The above tested the effect of N377L elimination from the full set and whether N377L stabilizes.

The set of mutations referred to as F5 are: V68I, S115V, A204L, V208L, V255W, N377L, and H66S.

Addition of W69L to minimal set may be done as previous work suggests aromatic residues in position 69 are destabilizing and is tested here.

The set of mutations referred to as F6 are: V68I, S115V, A204L, V208L, V255L, and W69L.

Using W69V instead of W69L may be done to test whether side chain length alters potential stabilizing effect.

The set of mutations referred to as F7 are: V68I, S115V, A204L, V208L, V255L, and W69V.

Using W69A instead of W69L/V may be done to further test whether side chain length alters potential stabilizing effect.

The set of mutations referred to as F8 are: V68I, S115V, A204L, V255L, V208L, and W69A.

Reintroduction of M426W may be done to test a minimally reduced set and the effect of M's.

The set of mutations referred to as F9 are: V68I, S115V, A204L, V208L, V255W, N377L, M426W, and H66S.

Reintroduction of M434W may be done to test a minimally reduced set and the effect of M's.

The set of mutations referred to as F10 are: V68I, S115V, A204L, V208L, V255W, N377L, M434W, and H66S.

Introduction of additional H72P mutation may be done to test if P can favor loop turn stabilizing TRP69 Loop in the W bound state.

The set of mutations referred to as F11 are: V68I, S115V, A204V, V208L, V255L, H72P, and H66S.

Testing minimal set with H66K rather than S may be done if the charge is a better solution to polar switch.

The set of mutations referred to as F12 are: V68I, S115V, V208L, V255L, and H66K.

Elimination of H66S from F1 may be done though H66 may be important for loop configuration.

The set of mutations referred to as F13 are: V68I, S115V, A204L, V208L, V255W, N377L, M426W, and M434W.

The Minimal Set 2 may include the elimination of H66S and swapping of S115V for A204V; H66 could be important for loop and A204 my better stabilize that S115V.

The set of mutations referred to as F14 are: V68I, A204V, V208L, and V255L.

Minimal Set 3 may include adding N377L to test for further stabilization.

The set of mutations referred to as F15 are: V68I, A204L, V208L, V255W, and N377L.

V3 Lock—Full Set

The set of mutations referred to as VT1 are: Y177F, T320L, D180A, Q422L, Y435F, Q203M, E381L, R298M, N302L, and N300L.

Elimination of R298M and E381L may be used to determine whether these two are stabilizing rather than destabilizing.

The set of mutations referred to as VT2 are: Y177F, T320L, D180A, Q422L, Y435F, Q203M, N302L, and N300L.

Elimination of E381L may be used to determine whether this residue is required to stabilize R298.

The set of mutations referred to as VT3 are: Y177F, T320L, D180A, Q422L, Y435F, Q203M, R298M, N302L, and N300L.

Elimination of R298M may be used to determine whether this reside stabilizes E381.

The set of mutations referred to as VT4 are: Y177F, T320L, D180A, Q422L, Y435F, Q203M, E381L, N302L, and N300L.

Retention of Y177F and Y435F may stabilize interior through H-bonding.

The set of mutations referred to as VT5 are: T320L, D180A, Q422L, Q203M, E381L, R298M, N302L, and N300L.

Retention of Y177F and Y435F while eliminating R298M and E381L mutations may be a minimal set avoiding possible problems from charged pair mutations.

The set of mutations referred to as VT6 are: T320L, D180A, Q422L, Q203M, N302L, N300L.

The Dennis Burton Set is a control for comparison.

The set of mutations referred to as VT7 are: R298A, N302F, R304V, A319Y, and T320M.

Elimination of D180A may be done as D180 appears to be destabilizing but may be stabilizing.

The set of mutations referred to as VT8 are: T320M, Q422M, Q203M, N302L, and N300L.

Addition of S174V may be done as S174 is on the periphery but may be stabilizing with a hydrophobe.

The set of mutations referred to as VT9 are: T320M, Q422M, Q203M, N302L, N300L, and S174V.

The Peter Kwong Set (DS-SOSIP.4mut) is an additional control set.

The set of mutations referred to as VT10 are: I201C, A443C, L154M, N300M, N302M, and T320L.

*In the above description, “elimination” means that F#N construct includes all F#N−1 mutations except the mutations identified as eliminated. In some embodiments, “retention” means the identified mutation is included.

Subsets of the mutations within a set are also contemplated. In a non-limiting embodiment, the mutations in Set F14 could be further parsed out to determine if there are fewer mutations or combinations of fewer mutations than in Set 14 which provide stabilization of the trimer.

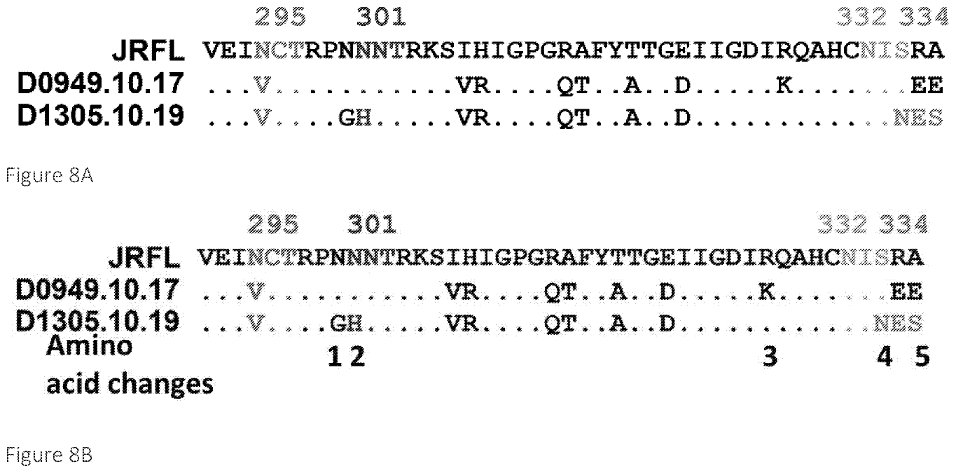

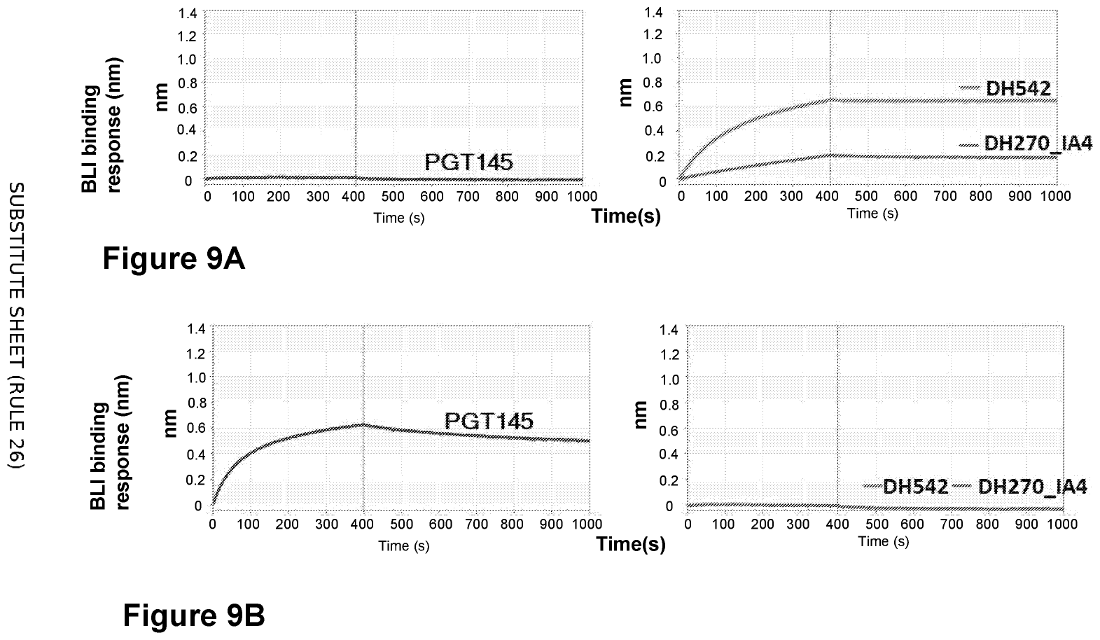

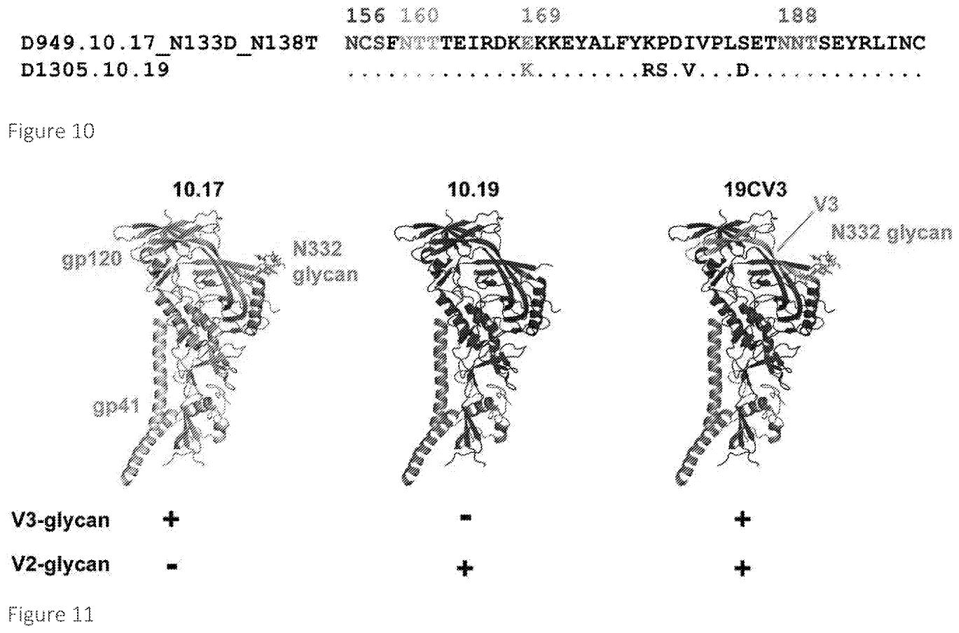

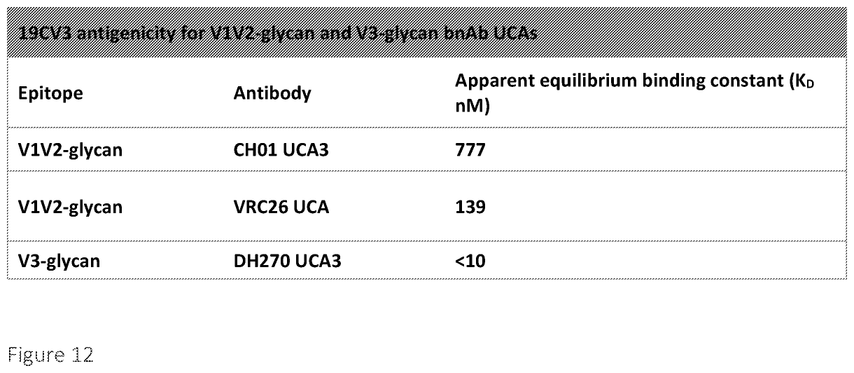

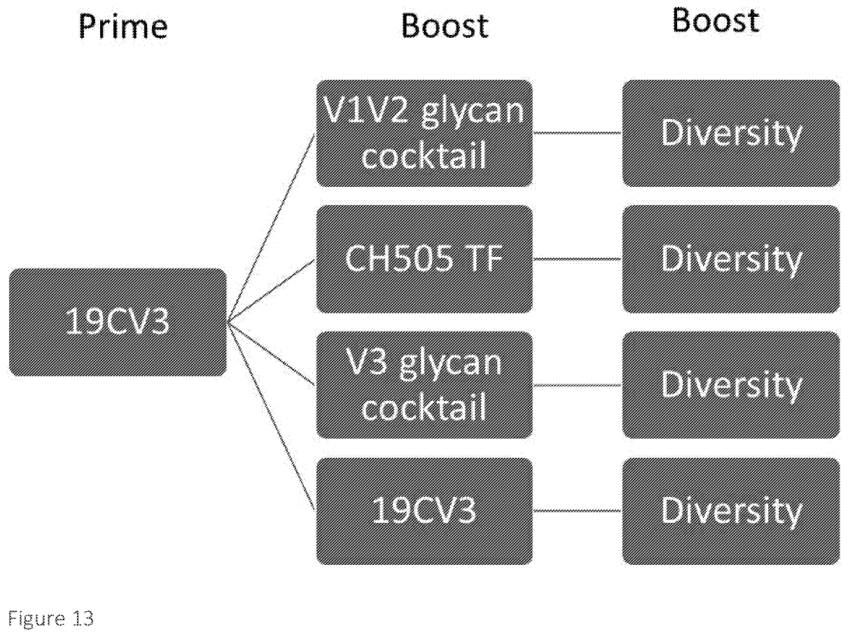

In certain embodiments the invention provides an envelope comprising 17aa V1 region without N133 and N138 glycosylation, and N301 and N332 glycosylation sites, and further comprising “GDIR” motif see Ex. 1 FIG. 8B, wherein the envelope binds to UCAs of V1V2 Abs and V3 Abs.

### Example 1: Pan-bnAb-Engaging Immunogens

This example describes design of HIV-1 envelopes antigenic for cross-epitope bnAb UCAs.

The discovery of broadly neutralizing antibodies (bnAbs) in HIV-1 infected individuals has provided evidence that the human immune system can target highly conserved epitopes on HIV-1 envelope. However, bnAbs have not been reproducibly induced with a vaccine in primates. One approach to improve the induction of bnAbs is to specifically design immunogens that bind to the precursor B cell that gives rise to the bnAb. While highly affinity matured HIV-1 bnAbs react with many Envelope proteins, their precursors bind only to select Envs. Currently, immunogens exist that can bind to a single bnAb precursor. These Envs have the disadvantage of relying on a single bnAb precursor to be present in most individuals. If the bnAb precursor antibody is not present in that individual, then the vaccine will not have the intended effect of inducing a specific type of antibody response. To improve the chances that an individual has the bnAb precursor that can engage the vaccine immunogen, we created a vaccine immunogen that can bind to multiple bnAb precursors. We designed the immunogen to interact with bnAbs precursors that interact with the first and second variable loop and glycans proximal to this loop—an epitope called V1V2-glycan. Secondly, the immunogen was also designed to interact with a bnAb precursor that bound to the third variable region and surrounding glycans on HIV-1 envelope—the V3-glycan site.

The immunogen was designed by creating a chimera of two HIV-1 envelope sequences that were derived from the HIV-1 infected individual CH0848 (See WO/2017152146 and WO/2018161049). The first Env CH0848.3.D0949.10.17 is antigenic for V3-glycan antibodies and was selected because it had a short first variable region in Env and bound to a V3-glycan antibody that possessed only 5 mutations (Bonsignori et al STM 2017). We modified this Env by removing glycosylation sites at 133 and 138 and found V3-glycan antibodies bound better to the Env when the glycosylation site was removed. These two glycosylation sites were identified as inhibitory in a neutralization screen where glycosylation sites on Env were removed to determine which glycans were required for neutralization by V3-glycan antibodies. For the CH0848.3.D0949.10.17 envelope we removed the glycosylation by substituting asparagine for amino acids that normally occur at positions 133 and 138 in other viruses. This glycan-modified Env bound with low nanomolar affinity to the V3-glycan bnAb precursor DH270 UCA3. To determine if a similar Env may have been present in the infected individual and could have potentially initiated the V3-glycan lineage in vivo, we screened all of the autologous virus sequences isolated from the infected individual CH0848 for viruses with a 17 amino acid variable region 1 and no glycans within the variable region except at position 156. We identified two sequences, with these characteristics. The first sequence CH0848.3.D1305.10.19 was produced as a recombinant protein. In biolayer interferometry assays it did not bind to V3-glycan antibodies. We created a pseudovirus expressing this Env and also found that V3 glycan antibodies did not neutralize it. However, we found that V1V2-glycan antibodies could bind to the recombinant protein. This was in contrast to CH0848.3.D0949.10.17 which lacked binding to V1V2-glycan bnAbs and precursors but was antigenic for V3-glycan antibodies. We inspected the sequences of the V1V2 and V3 regions and found that CH0848.3.D1305.10.19 lacked three glycans at positions 295, 301, and 332 usually bound by V3-glycan antibodies. To restore these V3 proximal glycosylation sites in CH0848.3.D1305.10.19 we used the V3 sequence of CH0848.3.D0949.10.17—the new envelope referenced as 19CV3. The modification of the CH0848.3.D1305.10.19 sequence to 19CV3 resulted in the addition of glycosylation sites at positions 301 and 332. We again made a recombinant protein of the chimeric envelope and found it bound to V1V2-glycan bnAbs as well as V3-glycan bnAbs—a combination of the phenotypes of the two parental envelopes. We next tested the binding of the bnAb precursors for V1V2 and V3-glycan sites. We found that 19CV3 bout to the bnAb precursor for two V1V2 glycan bnAb, CHO1 and VRC26, and V3 glycan Ab DH270.

With reference to CH0848 10.17DT SOSIP sequence see WO2018/161049, incorporated by reference in its entirety.

For non-limiting examples of hole-filled CH848 703010848.3.d0949.10.17envelopes, see WO/2017152146 and WO2018/161049, inter alia without limitation, FIGS. 44A-D and paragraph [0091], incorporated by reference in its entirety.

The immunogens of the invention can be delivered by any suitable mechanism.

In non-limiting embodiments, theses could be Adeno-associated virus (AAV) vectors. Characteristics of AAVs may include:
Being non-replicating viral vectors;
Providing sustained expression of the immunogen;
The ability to transduce dendritic cells, which present transgene(immunogen) in complex with MHCII to naïve T cells;
Constant antigen production which could lead to improved clonal persistence, enhanced germinal center reactions, and higher somatic mutation; and
Can be used a multivalent mixture to mimic chronic HIV-1 infection.

In certain embodiments, the immunogens could be multimerized.

Any of the inventive envelope designs could be tested functionally in any suitable assay. Non-limiting assays including analysis of antigenicity or immunogenicity.

### Example 2 Animal Study

19CV3 SOSIP trimer was used to immunize non-human primates.

Design of NHP Study Using 19CV3

| | Animal | | | Binding | Neutralizing |
|---|---|---|---|---|---|
| Study # | Model | Synopsis | Adjuvant | antibody | antibody |
| NHP158 | Rhesus | 4X 19CV3 every 4 | GLA-SE | TBD | TBD |
| | | weeks | | | |

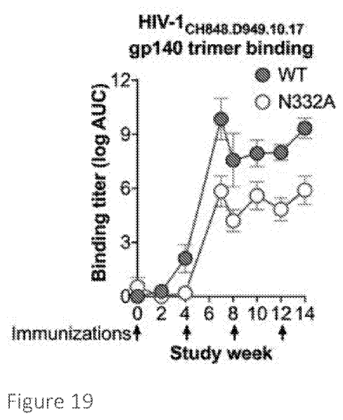

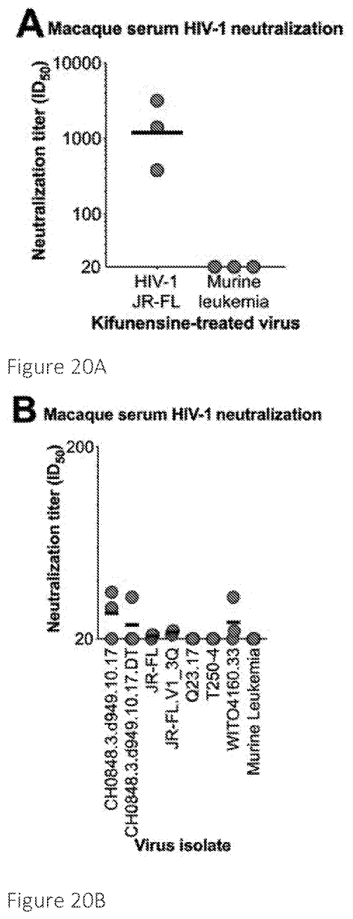

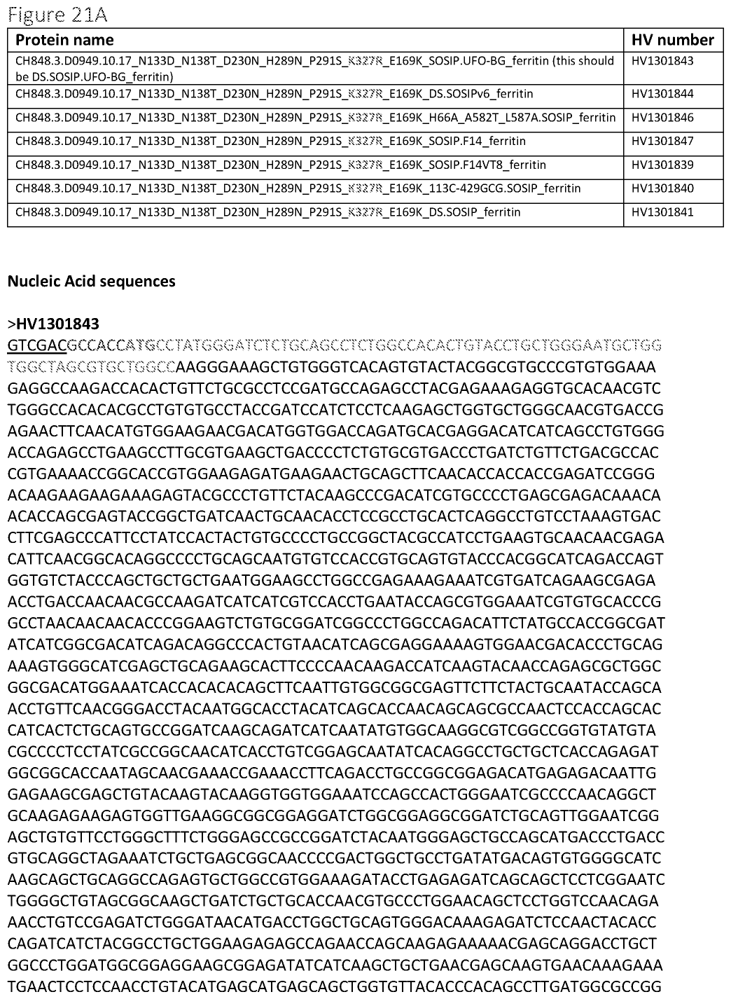

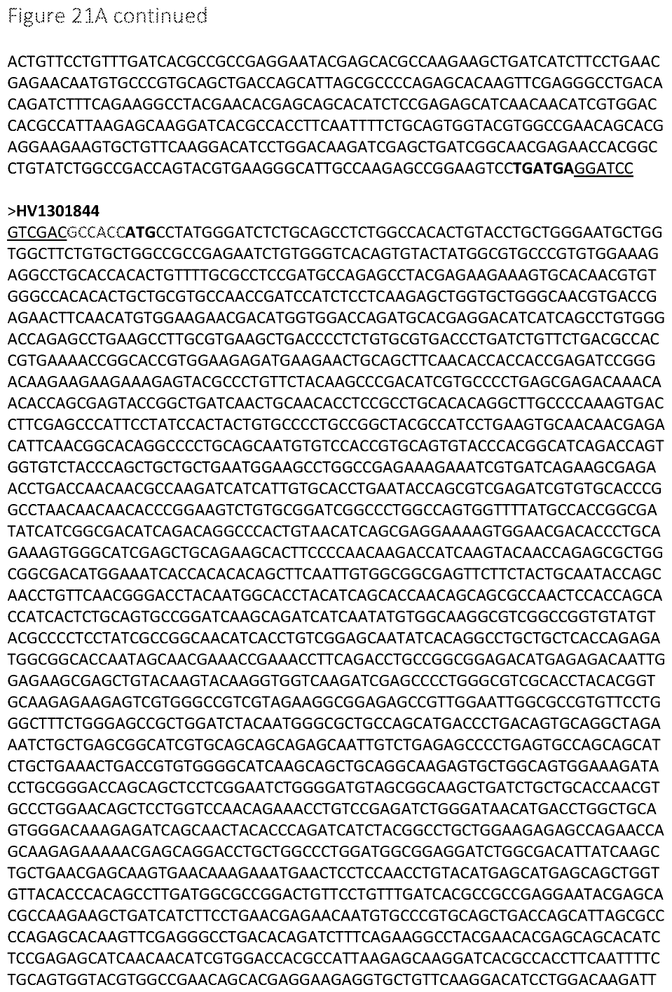

FIGS. 19-20 show data from NHP study #158.

What is claimed is:

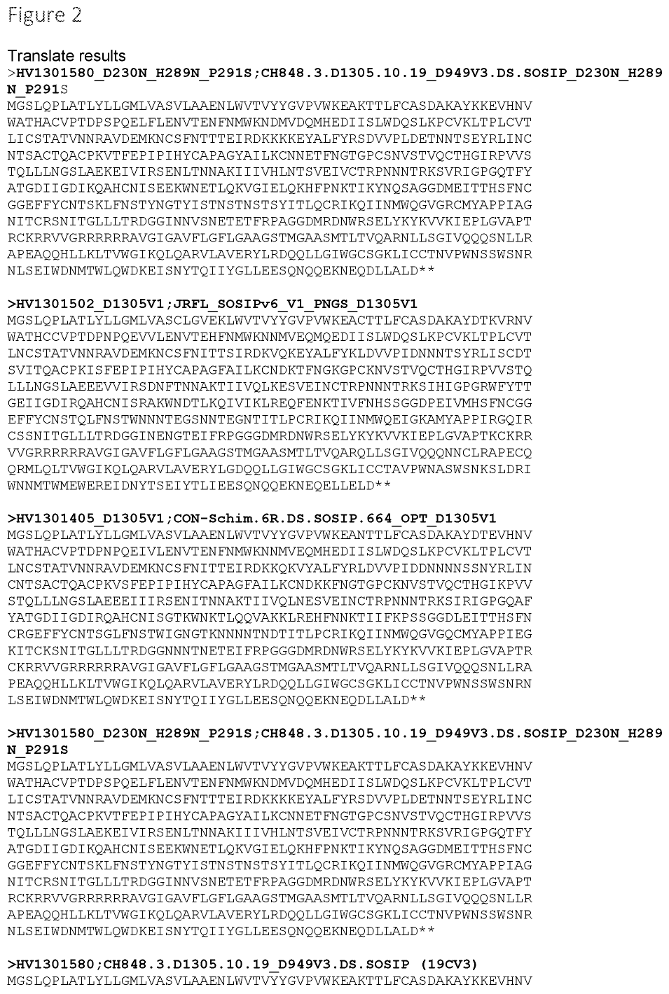

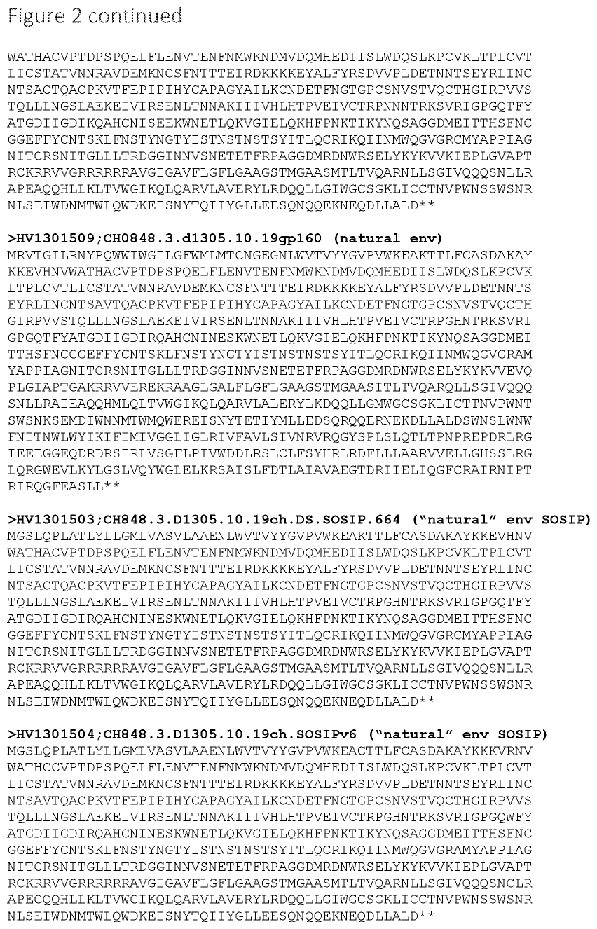

1. A recombinant HIV-1 envelope selected from the envelopes listed in FIG. 1, FIG. 2, FIG. 3, or FIGS. 21-25.

2. A composition comprising the envelope of claim 1 and a carrier, wherein the envelope is a protomer comprised in a trimer.

3. The composition of claim 2 wherein the envelope is the envelope is comprised in a stable trimer.

4. A composition comprising a nanoparticle and a carrier, wherein the nanoparticle comprises any one of the envelopes of claim 1.

5. The composition of claim 4, wherein the nanoparticle is ferritin self-assembling nanoparticle.

6. A composition comprising a nanoparticle and a carrier, wherein the nanoparticle comprises any one of the trimers of claim 2 or 3.

7. The composition of claim 6 wherein the nanoparticle is ferritin self-assembling nanoparticle.

8. The composition of claim 7 wherein the nanoparticle comprises multimers of trimers.

9. The composition of claim 7 wherein the nanoparticle comprises 1-8 trimers.

10. A method of inducing an immune response in a subject comprising administering an immunogenic composition comprising any one of the recombinant envelopes the preceding claims or compositions of the preceding claims.

11. The method of claim 10 wherein the composition is administered as a prime.

12. The method of claim 10 wherein the composition is administered as a boost.

13. A nucleic acid encoding any of the recombinant envelopes of the preceding claims.

14. A composition comprising the nucleic acid of claim 13 and a carrier.

15. A method of inducing an immune response in a subject comprising administering an immunogenic composition comprising the nucleic acid of claim 13 or the composition of claim 14.


SEQUENCE LISTING (96 sequences)

SEQ ID NO: 1 - protein, 4 aa, Artificial Sequence
  Description of Artificial Sequence Synthetic peptide
GDIR

SEQ ID NO: 2 - protein, 4 aa, Artificial Sequence
  Description of Artificial Sequence Synthetic peptide
GDIK

SEQ ID NO: 3 - DNA, 1947 bp, Artificial Sequence
  Description of Artificial Sequence Synthetic polynucleotide
atgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 4 - DNA, 1932 bp, Artificial Sequence
  Description of Artificial Sequence Synthetic polynucleotide
atgggatctctgcagcctctggccacactgtacctgctgggaatgctggtggcctcttgtctgggcgttgagaagctgtgggtcaccgtgtactatggcgtgcccgtgtggaaagaagcctgcaccacactgttctgtgccagcgacgccaaggcctacgataccaaggtgcgcaatgtgtgggccactcactgctgcgtgcccaccgatcctaatcctcaagaggtggtgctggaaaacgtgaccgagcacttcaacatgtggaagaacaacatggtcgagcagatgcaagaggacatcatcagcctgtgggaccagagcctgaagccttgcgtgaagctgacccctctgtgcgtgaccctgaactgtagcaccgccaccgtgaacaacagagccgtggacgagatgaagaactgcagcttcaacatcaccacctccatcagagacaaggtgcagaaagagtacgccctgttctacaagctggacgtggtgcccatcgacaacaacaacaccagctacagactgatcagctgcgacaccagcgtgatcacccaggcctgtcctaagatcagcttcgagcccattcctatccactactgtgcccctgccggcttcgccatcctgaagtgcaacgacaagaccttcaacggcaagggcccctgcaagaacgtgtccaccgtgcagtgtacccacggcatcagaccagtggtgtctacccagctgctgctgaatggctctctggccgaggaagaagtggtcatcagaagcgacaacttcaccaacaacgccaagaccatcatcgtgcagctgaaagagagcgtcgagatcaactgcacccggcctaacaacaatacccggaagtccatccacatcggccctggcagatggttttacaccaccggcgagattatcggcgacatcagacaggcccactgcaacatcagccgggccaagtggaacgacaccctgaagcagatcgtgatcaagctgagagagcagttcgagaacaagacgatcgtgttcaaccacagctctggcggcgaccccgagattgtgatgcactcctttaactgtggcggcgagttcttctactgcaacagcacccagctgttcaactccacctggaacaacaacacagagggcagcaacaataccgagggcaacaccatcacactgccctgccggatcaagcagatcatcaatatgtggcaagagatcggcaaggctatgtacgcccctcctatccggggccagatcagatgcagcagcaatatcacaggcctgctgctcaccagagatggcggcatcaacgagaacggcaccgagatcttcagacccggcggaggcgacatgcgggacaattggagaagcgagctgtacaagtacaaggtggtcaagatcgagcccctgggcgtcgcccctaccaagtgcaagagaagagtcgtgggccgtagaaggcggcggagagctgttggaattggcgccgtgttcctgggctttctgggagccgctggatctacaatgggcgctgccagcatgaccctgacagtgcaggctagacagctgctgtccggcattgtccagcagcagaacaactgcctgagagcccctgagtgtcaacaaagaatgctgcagctgaccgtgtggggcatcaaacagctgcaggccagagttctggccgtggaaagatacctgggcgaccagcagctcctcggcatctggggatgttctggcaagctgatttgctgcaccgccgtgccttggaatgccagctggtctaacaagagcctggaccggatctggaacaatatgacctggatggaatgggagcgcgagatcgacaactacacctccgagatctacaccctgatcgaggaaagccagaaccagcaagagaaaaacgagcaagagctgctcgagctggattaatga

SEQ ID NO: 5 - DNA, 1944 bp, Artificial Sequence
  Description of Artificial Sequence Synthetic polynucleotide
atgggctcgctccagccgctcgcgacgctgtacctcctgggcatgctcgtggcgtccgtgctggcggccgagaacctgtgggtgacggtgtactacggcgtgcccgtgtggaaggaggccaacaccacgctgttctgcgccagcgacgccaaggcctacgacaccgaggtgcacaacgtgtgggcgacccacgcctgcgtgccgacggaccccaacccccaggagatcgtgctggagaacgtgaccgagaacttcaacatgtggaagaacaacatggtggagcagatgcacgaggacatcatctcgctgtgggaccagtccctgaagccgtgcgtgaagctgacgcccctgtgcgtgaccctgaactgtagcaccgccaccgtgaacaacagagccgtggacgagatgaagaactgctccttcaacatcaccaccgagatccgcgacaagaagcagaaggtgtacgcgctgttctaccggctggacgtggtgccgatcgacgacaacaacaacaactccagcaactaccgcctgatcaactgcaacaccagcgcctgcacccaggcctgcccgaaggtgtccttcgagcccatccccatccactactgcgcgccggccggcttcgccatcctgaagtgcaacgacaagaagttcaacggcaccggcccctgcaagaacgtgtccaccgtgcagtgcacccacgggatcaagcccgtggtgtccacgcagctgctgctgaacggctccctggccgaggaggagatcatcatccgctccgagaacatcacgaacaacgccaagaccatcatcgtgcagctgaacgagtccgtggagatcaactgcaccaggcccaacaacaacacccgcaagtccatccggatcggccctggccaggcgttctacgccaccggcgacatcatcggcgacatccgccaggcgcactgcaacatctcgggcacgaagtggaacaagaccctgcagcaggtggcgaagaagctgcgcgagcacttcaacaacaagaccatcatcttcaagcccagctccggcggcgacctggagatcacgacccactccttcaactgccgcggcgagttcttctactgcaacacctccggcctgttcaactcgacgtggatcgggaacggcacgaagaacaacaacaacaccaacgacaccatcaccctgccctgccgcatcaagcagatcatcaacatgtggcagggcgtgggccagtgcatgtacgcgccgcccatcgagggcaagatcacctgcaagtccaacatcaccggcctgctcctgacgcgcgacggcggcaacaacaacaccaacgagaccgagatcttcaggccgggcggcggcgacatgcgcgacaactggcgctcggagctgtacaagtacaaggtggtgaagatcgagcccctgggcgtggcgccgacgcgctgcaagagacgcgtggtgggccgcagacgaaggagacgggccgtgggcatcggcgcggtgttcctgggcttcctgggagcagctggttcgacgatgggcgcagcttccatgaccctgacagtgcaggcacgcaacctgctctccggcatcgtccagcagcagtcgaacctgcttcgagcccccgaggcgcagcagcacctcctcaagctgaccgtgtggggcatcaagcagctgcaggcacgcgtgctagccgtggagcgctacctccgcgaccagcagctgctcggaatctggggctgctcgggcaagctgatctgctgcaccaacgtgccgtggaacagctcctggtccaaccgcaacctctcggagatctgggacaacatgacctggctccagtgggacaaggagatctcgaactacacccagatcatctacggcctgctggaggagtcccagaaccagcaggagaagaacgagcaggacctgctggccctggactgataa

SEQ ID NO: 6 - DNA, 1947 bp, Artificial Sequence
  Description of Artificial Sequence Synthetic polynucleotide
atgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 7 - DNA, 2559 bp, Artificial Sequence
  Description of Artificial Sequence Synthetic polynucleotide
atgagagtgacggggatactgaggaattatccacaatggtggatatggggcatcttaggcttttggatgctaatgacttgtaatggggaaggaaacttgtgggtcacagtctactatggggtaccagtatggaaagaagcaaaaactactctgttttgtgcatcagatgccaaagcgtataagaaagaagtgcataatgtctgggcgacacatgcctgtgtacccacagaccccagcccacaagaactgtttttggaaaatgtaacagaaaattttaacatgtggaaaaatgatatggtagatcagatgcatgaggatataatcagtctatgggatcaaagcctaaagccatgtgtaaagttgaccccactctgtgtcactttaatttgtagtactgctactgttaacaatagagcagttgatgaaatgaaaaattgctccttcaatacaaccacagaaataagagataagaaaaagaaggaatatgcacttttttatagatctgatgtagtaccacttgatgaaacaaacaataccagtgagtatagattaataaattgtaatacctcagccgtaacacaagcctgtccaaaggtcacttttgaaccaattcctatacattattgtgctccagctggttatgcgattctaaagtgcaatgatgagacattcaatggaacaggaccatgcagtaatgtcagcacagtacaatgtacacatggaattaggccagtggtatcaacccaactactgttaaatggtagtctggcagaaaaagagatagtaattagatctgaaaacctgacaaacaatgccaaaataataatagtccatcttcacacccctgtagaaattgtgtgtacaaggcccggccataatacaaggaaaagtgtgagaataggcccaggacaaacattctatgcaacaggagacataataggagatataagacaagcacattgtaacattaatgaaagtaaatggaatgaaactttacaaaaggtaggtatagaattgcaaaaacacttccctaataagacaataaagtataaccaatccgcaggaggagacatggaaattacaacacatagctttaattgtggaggagaatttttctattgcaatacatcaaagctgtttaatagtacatacaatggtacatacataagtacaaacagcacaaacagtacttcatacatcacgcttcaatgcagaataaaacaaattataaacatgtggcagggggtaggaagagcaatgtatgctcctcccattgcaggaaacataacatgtagatcaaatatcacagggctactattgacacgtgatggagggatcaataatgttagcaacgagacagagacattcaggcctgcaggaggagatatgagggataattggagaagtgaattatataaatataaagtagtagaagttcagccattaggaatagcaccaactggtgcaaaaaggagagtggtggagagagaaaaaagagcagcaggactaggagctttgttccttgggttcttgggagcagcaggaagcactatgggcgcagcatcaataacgctgacggtacaggccagacaattgttgtctggtatagtgcaacagcaaagcaatttgctgagggctatagaggcgcaacagcatatgttgcaactcacggtctggggcatcaagcagctccaggcaagagtcctggctctggaaagatacctaaaggatcaacagctcctagggatgtggggctgctctggaaaactcatctgcaccactaatgtgccttggaacactagttggagtaataaatctgaaatggatatttggaataacatgacatggatgcagtgggaaagagaaattagcaattacacagagacaatatacatgttgcttgaagactcgcaacgccagcaggagagaaatgaaaaagatttactagcattggacagttggaacagtctgtggaattggtttaacataacaaactggctgtggtatataaaaatattcataatgatagtagggggcttgataggtttaagaatagtttttgctgtgctatctatagtgaatagagtcaggcagggatactcacctttgtcgttgcagacccttaccccaaacccgagggaacccgacaggctcagaggaatcgaagaagaaggtggagagcaagacagagacagatccattcgattagtgagcggattcttgccaattgtctgggacgacctgcggagcctgtgcctcttcagttaccaccgattgagagactttctattgctggcagcgagagtggtggaacttctgggacacagcagtctcaggggactgcagagggggtgggaagtccttaagtatctgggaagtcttgtgcagtattggggtctggaactaaaaaggagtgctattagtctttttgataccctagcaatagcagtagctgaaggaacagataggattatagaattaatacaaggattttgtagagctatccgcaacatacctacaagaataagacaaggctttgaagcatctttgctataataa

SEQ ID NO: 8 - DNA, 1947 bp, Artificial Sequence
  Description of Artificial Sequence Synthetic polynucleotide
atgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcaccagacctggccacaacacccggaagtctgtgcggattggacccggccagaccttttatgccaccggcgatatcatcggcgacatcagacaggcccactgtaacatcaacgagagcaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagatgcatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 9 - DNA, 1947 bp, Artificial Sequence
  Description of Artificial Sequence Synthetic polynucleotide
atgggatctctgcagcctctggccacactgtacctgctgggaatgctggtggcttctgtgctggccgccgagaatctgtgggtcacagtgtactatggcgtgcccgtgtggaaagaggcctgtaccacactgttctgtgccagcgacgccaaggcctacaagaaaaaggtgcgcaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcaccagacctggccacaacacccggaagtctgtgcggattggacccggccagtggttttatgccaccggcgatatcatcggcgacatcagacaggcccactgtaacatcaacgagagcaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaattgcctcagagcccctgagtgtcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggactgataa

SEQ ID NO: 10 - protein, 647 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 11 - protein, 642 aa, Artificial Sequence
  Description of Artificial Sequence Synthetic polypeptide
MGSLQPLATLYLLGMLVASCLGVEKLWVTVYYGVPVWKEACTTLFCASDAKAYDTKVRNVWATHCCVPTDPNPQEVVLENVTEHFNMWKNNMVEQMQEDIISLWDQSLKPCVKLTPLCVTLNCSTATVNNRAVDEMKNCSFNITTSIRDKVQKEYALFYKLDVVPIDNNNTSYRLISCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRWFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEIGKAMYAPPIRGQIRCSSNITGLLLTRDGGINENGTEIFRPGGGDMRDNWRSELYKYKVVKIEPLGVAPTKCKRRVVGRRRRRRAVGIGAVFLGFLGAAGSTMGAASMTLTVQARQLLSGIVQQQNNCLRAPECQQRMLQLTVWGIKQLQARVLAVERYLGDQQLLGIWGCSGKLICCTAVPWNASWSNKSLDRIWNNMTWMEWEREIDNYTSEIYTLIEESQNQQEKNEQELLELD

SEQ ID NO: 12 - protein, 646 aa, Artificial Sequence
  Description of Artificial Sequence Synthetic polypeptide
MGSLQPLATLYLLGMLVASVLAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCSTATVNNRAVDEMKNCSFNITTEIRDKKQKVYALFYRLDVVPIDDNNNNSSNYRLINCNTSACTQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQCMYAPPIEGKITCKSNITGLLLTRDGGNNNTNETEIFRPGGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 13 - protein, 647 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 14 - protein, 851 aa, Artificial Sequence
  Description of Artificial Sequence Synthetic polypeptide
MRVTGILRNYPQWWIWGILGFWMLMTCNGEGNLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLALERYLKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGYSPLSLQTLTPNPREPDRLRGIEEEGGEQDRDRSIRLVSGFLPIVWDDLRSLCLFSYHRLRDFLLLAARVVELLGHSSLRGLQRGWEVLKYLGSLVQYWGLELKRSAISLFDTLAIAVAEGTDRIIELIQGFCRAIRNIPTRIRQGFEASLL

SEQ ID NO: 15 - protein, 647 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQTFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 16 - protein, 647 aa, Artificial Sequence
  Description of Artificial Sequence Synthetic polypeptide
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPGHNTRKSVRIGPGQWFYATGDIIGDIRQAHCNINESKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALD

SEQ ID NO: 17 - protein, 6 aa, Artificial Sequence
  Description of Artificial Sequence Synthetic peptide
LPSTGG

SEQ ID NO: 18 - protein, 655 aa, Artificial Sequence
  Description of Artificial Sequence Synthetic polypeptide
MPMGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDLPSTGG

SEQ ID NO: 19 - DNA, 1989 bp, Artificial Sequence
  Description of Artificial Sequence Synthetic polynucleotide
gtcgacgccaccatgcccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacctgcctagcaccggaggatgataaggatcc

SEQ ID NO: 20 - protein, 30 aa, Artificial Sequence
  Description of Artificial Sequence Synthetic polypeptide
CVKLTPLCVTLICSDATVKTGTVEEMKNCS

SEQ ID NO: 21 - protein, 30 aa, Artificial Sequence
  Description of Artificial Sequence Synthetic polypeptide
CVKLTPLCVTLICSNATVKNGTVEEMKNCS

SEQ ID NO: 22 - protein, 30 aa, Artificial Sequence
  Description of Artificial Sequence Synthetic polypeptide
CVKLTPLCVTLICSTATVNNRAVDEMKNCS

SEQ ID NO: 23 - protein, 44 aa, Artificial Sequence
  Description of Artificial Sequence Synthetic polypeptide
VEINCTRPNNNTRKSIHIGPGRAFYTTGEIIGDIRQAHCNISRA

SEQ ID NO: 24 - protein, 44 aa, Artificial Sequence
  Description of Artificial Sequence Synthetic polypeptide
VEIVCTRPNNNTRKSVRIGPGQTFYATGDIIGDIKQAHCNISEE

SEQ ID NO: 25 - protein, 44 aa, Artificial Sequence
  Description of Artificial Sequence Synthetic polypeptide
VEIVCTRPGHNTRKSVRIGPGQTFYATGDIIGDIRQAHCNINES

SEQ ID NO: 26 - protein, 43 aa, Artificial Sequence
  Description of Artificial Sequence Synthetic polypeptide
NCSFNTTTEIRDKEKKEYALFYKPDIVPLSETNNTSEYRLINC

SEQ ID NO: 27 - protein, 43 aa, Artificial Sequence
  Description of Artificial Sequence Synthetic polypeptide
NCSFNTTTEIRDKKKKEYALFYRSDVVPLDETNNTSEYRLINC

SEQ ID NO: 28 - DNA, 2445 bp, Artificial Sequence
  Description of Artificial Sequence Synthetic polynucleotide
gtcgacgccaccatgcctatgggatctctgcagcctctggccacactgtacctgctgggaatgctggtggctagcgtgctggccaagggaaagctgtgggtcacagtgtactacggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtggaaatccagccactgggaatcgccccaacaggctgcaagagaagagtggttgaaggcggcggaggatctggcggaggcggatctgcagttggaatcggagctgtgttcctgggctttctgggagccgccggatctacaatgggagctgccagcatgaccctgaccgtgcaggctagaaatctgctgagcggcaaccccgactggctgcctgatatgacagtgtggggcatcaagcagctgcaggccagagtgctggccgtggaaagatacctgagagatcagcagctcctcggaatctggggctgtagcggcaagctgatctgctgcaccaacgtgccctggaacagctcctggtccaacagaaacctgtccgagatctgggataacatgacctggctgcagtgggacaaagagatctccaactacacccagatcatctacggcctgctggaagagagccagaaccagcaagagaaaaacgagcaggacctgctggccctggatggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagatcgagctgatcggcaacgagaaccacggcctgtatctggccgaccagtacgtgaagggcattgccaagagccggaagtcctgatgaggatcc

SEQ ID NO: 29 - DNA, 2472 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggcctgcaccacactgttttgcgcctccgatgccagagcctacgagaagaaagtgcacaacgtgtgggccacacactgctgcgtgccaaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcacctgaataccagcgtcgagatcgtgtgcacccggcctaacaacaacacccggaagtctgtgcggatcggccctggccagtggttttatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgtctgagagcccctgagtgccagcagcatctgctgaaactgaccgtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaggtgctgttcaaggacatcctggacaagattgagctgatcggcaacgagaaccacggcctgtatctggccgaccagtacgtgaagggaatcgccaagagcagaaagagctgatgaggatcc

SEQ ID NO: 30 - DNA, 2472 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtggccaacgtttgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcacctgaataccagcgtcgagatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggccagagtgctgacagtggaaagatacgccagggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggctctggatggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 31 - DNA, 2472 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaagtgtgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtgggcagagctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagctccaacctgtacatgagcatgtccagctggtgctacacccacagtcttgatggcgccggactgttcctgtttgaccacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 32 - DNA, 2472 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacatctgggccacacacgcctgcgtgccaaccgatccatctcctcaagaactggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacaagcgccgtgacaatggtctgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtccacctgaacacctccgtggaaatcgtgtgcacccggcctctgaatctgaccagaaagagcgtgcggatcggccctggccagaccttttatgccatgggcgacatcatcggcgacatccggcaggcccactgcaacatctctgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagatgatcattaacatgtggcaaggcgtcggcagggctatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgatatcatcaagctgctgaacgagcaagtgaacaaagaaatgaacagctccaacctgtacatgagcatgtccagctggtgctacacccacagtcttgatggcgccggactgttcctgtttgaccacgccgccgaggaatacgagcacgccaagaagctgatcattttcctgaacgagaacaacgtgccagtgcagctgaccagcattagcgccccagagcacaagttcgagggcctgacacagatctttcagaaggcctacgaacacgagcagcacatcagcgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 33 - DNA, 2478 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtggtgccagtctctgaagccctgcgtgaagctgacccctctgtgtgtgaccctgatctgctctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggctgcggcgtgggcagagctatgtatgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgttgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 34 - DNA, 2472 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcagacaggcccactgtaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 35 - protein, 807 aa, Artificial Sequence
  Description of Artificial Sequence Synthetic polypeptide
MPMGSLQPLATLYLLGMLVASVLAKGKLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVEIQPLGIAPTGCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 36 - protein, 816 aa, Artificial Sequence
  Description of Artificial Sequence Synthetic polypeptide
MPMGSLQPLATLYLLGMLVASVLAAENLWVTVYYGVPVWKEACTTLFCASDARAYEKKVHNVWATHCCVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 37 - protein, 816 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVANVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLTVERYARDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 38 - protein, 816 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 39 - protein, 816 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNIWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKMIINMWQGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 40 - protein, 818 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWCQSLKPCVKLTPLCVTLICSDATVKTGTVEEMKNCSFNTTTEIRDKKKKEYALFYKPDIVPLSETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGCGVGRAMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 41 - protein, 816 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIRQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 42 - DNA, 2532 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtggtggcggatctggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 43 - DNA, 2517 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtgatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 44 - DNA, 2502 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggagatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 45 - DNA, 2487 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcgatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 46 - DNA, 2541 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtggtggcggatctggactgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 47 - DNA, 2526 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggaggcggaggctcaggtctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 48 - DNA, 2511 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcggcggaggaagcggactgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 49 - DNA, 2496 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcggaggcggttctggcctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 50 - DNA, 2481 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgcgcctccgatgccagagcctacgagaaagaggtgcacaacgtctgggccacacacgcctgtgtgcctaccgatccatctcctcaagagctggtgctgggcaacgtgaccgagaacttcaacatgtggaagaacgacatggtggaccagatgcacgaggacatcatcagcctgtgggaccagagcctgaagccttgcgtgaagctgacccctctgtgcgtgaccctgatctgttctgacgccaccgtgaaaaccggcaccgtggaagagatgaagaactgcagcttcaacaccaccaccgagatccgggacaagaagaagaaagagtacgccctgttctacaagcccgacatcgtgcccctgagcgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtccacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgacaccctgcagaaagtgggcatcgagctgcagaagcacttccccaacaagaccatcaagtacaaccagagcgctggcggcgacatggaaatcaccacacacagcttcaattgtggcggcgagttcttctactgcaataccagcaacctgttcaacgggacctacaatggcacctacatcagcaccaacagcagcgccaactccaccagcaccatcactctgcagtgccggatcaagcagatcatcaatatgtggcaaggcgtcggccggtgtatgtacgcccctcctatcgccggcaacatcacctgtcggagcaatatcacaggcctgctgctcaccagagatggcggcaccaatagcaacgaaaccgaaaccttcagacctgccggcggagacatgagagacaattggagaagcgagctgtacaagtacaaggtggtcaagatcgagcccctgggcgtcgcacctacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggctgttctggcaagctgatctgctgcaccaacgtgccctggaacagctcctggtccaacagaaacctgtccgagatctgggataacatgacctggctgcagtgggacaaagagatcagcaactacacccagatcatctacggcctgctggaagagagccagaaccagcaagagaaaaacgagcaggacctgctggccctggatggcggaggatctggcctgagcaaggatatcatcaagctgctgaacgagcaagtgaacaaagaaatgaactcctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagtccaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 51 - protein, 836 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 52 - protein, 831 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 53 - protein, 826 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 54 - protein, 821 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 55 - protein, 839 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 56 - protein, 834 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 57 - protein, 829 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 58 - protein, 824 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 59 - protein, 819 aa, Artificial Sequence
  Description of Artificial Sequence Synthetic polypeptide
MPMGSLQPLATLYLLGMLVASVLAAENLWVTVYYGVPVWKEAKTTLFCASDARAYEKEVHNVWATHACVPTDPSPQELVLGNVTENFNMWKNDMVDQMHEDIISLWDQSLKPCVKLTPLCVTLICSDATVKTGTVEEMKNCSFNTTTEIRDKKKKEYALFYKPDIVPLSETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNDTLQKVGIELQKHFPNKTIKYNQSAGGDMEITTHSFNCGGEFFYCNTSNLFNGTYNGTYISTNSSANSTSTITLQCRIKQIINMWQGVGRCMYAPPIAGNITCRSNITGLLLTRDGGTNSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 60 - DNA, 2475 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacatctgggccacacacgcctgcgtgccaaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccatcaccatggtctgccccaagctgaccttcgagcccattcctatccactactgtgcccctgccggctacgccatcctgaagtgcaacgacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcattgtgcatctgcacacccctgtggaaatcgtgtgcacccggcctctgaatctgaccagaaagagcgtgcggatcggccctggccagaccttttatgccatgggcgacatcatcggcgatatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagatgatcattaacatgtggcaaggcgtcggcagggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggcatcaagcagctgcaggcaagagtgctggcagtggaaagatacctgcgggaccagcagctcctcggaatctggggatgtagcggcaagctgatctgctgcaccaacgtgccctggaacagcagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggcccttgatggtggtggaagcggagatatcatcaagctgctgaacgagcaagtgaacaaagaaatgaacagctccaacctgtacatgagcatgtccagctggtgttacacccacagcctggatggcgccggactgttcctgtttgatcacgccgccgaggaatacgagcacgccaagaagctgatcattttcctgaacgagaacaacgtgccagtgcagctgaccagcatctctgcccctgagcacaagttcgagggcctgacacagatctttcagaaggcctacgaacacgagcagcacatctccgagtccatcaacaatatcgtggaccacgccatcaagagcaaggatcacgccaccttcaactttctccagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 61 - DNA, 2475 bp, Artificial Sequence
  Description of Artificial Sequence Synthetic polynucleotide
gtcgacgccaccatgcccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 62 - DNA, 2562 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtggaagtgcagcccctgggaatcgctccaacaggcgctaagagaagagtggtggaacgcgagaaaagagccgctggactgggagccctgtttctgggttttctgggagccgccggaagcacaatgggagctgcctctatcacactgaccgtgcaggctagacagctgctgagcggaattgtgcagcagcagagcaacctgctgagagccattgaagcccagcagcacatgctgcagctgaccgtgtggggaatcaaacagctgcaggccagagtgctgaccctggaaagatacgccaaggaccagcagctcctcggcatgtggggatgttctggcaagctgatctgcaccaccaacgtgccctggaacacctcctggtccaacaagagcgagatggacatctggaacaacatgacctggatgcagtgggagagagagatcagcaactacaccgagacaatctacatgctgctcgaggacagccagcggcagcaagagagaaacgagaaggatctgctggccctggactcctggaatagcctgtggaactggttcaatatcaccaactggctgtggtacatcaagatcttcatcatgatcgtcggcggcctgatcggcctgagaatcgtgtttgccgtgctgagcatcgtgaacagagtgcggcagggaatcagcccactgagcctgcaaaccctgacacctaatcctagagagcccgaccggctgagaggcatcgaagaagaaggcggcgagcaggacagagacagatccatcagactggtgtccggcttcctgcctatcgtgtgggacgatctgagaagcctgtgcctgttcagctaccaccggctgcgggattttctgctgcttgccgccagagtggttgaactgctgggacacagctctctgcggggactgcaaagaggctgggaagtcctgaagtacctgggcagcctggtgcagtattggggcctcgagctgaagagaagcgccattagcctgttcgacaccctggccattgctgtggccgaaggcaccgacagaatcattgagctgatccagggcttctgccgggccatcagaaacatccctaccagaatccggcagggcttcgaggcttccctgctgtgataaggatcc

SEQ ID NO: 63 - protein, 655 aa, Artificial Sequence; identical to SEQ ID NO: 18
  Description of Artificial Sequence Synthetic polypeptide

SEQ ID NO: 64 - protein, 817 aa, Artificial Sequence
  Description of Artificial Sequence Synthetic polypeptide
MPMGSLQPLATLYLLGMLVASVLAAENLWVTVYYGVPVWKEAKTTLFCASDAKAYKKEVHNIWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAITMVCPKLTFEPIPIHYCAPAGYAILKCNDETFNGTGPCSNVSTVQCTHGIRPVLSTQLLLNGSLAEKEIVIRSENLTNNAKIIIVHLHTPVEIVCTRPLNLTRKSVRIGPGQTFYAMGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKMIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 65 - protein, 817 aa, Artificial Sequence
  Description of Artificial Sequence Synthetic polypeptide
MPMGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 66 - protein, 846 aa, Artificial Sequence
  Description of Artificial Sequence Synthetic polypeptide
MPMGSLQPLATLYLLGMLVASVLAEGNLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLTLERYAKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGISPLSLQTLTPNPREPDRLRGIEEEGGEQDRDRSIRLVSGFLPIVWDDLRSLCLFSYHRLRDFLLLAARVVELLGHSSLRGLQRGWEVLKYLGSLVQYWGLELKRSAISLFDTLAIAVAEGTDRIIELIQGFCRAIRNIPTRIRQGFEASLL

SEQ ID NO: 67 - DNA, 2223 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtggaagtgcagcccctgggaatcgctccaacaggcgctaagagaagagtggtggaacgcgagaaaagagccgctggactgggagccctgtttctgggttttctgggagccgccggaagcacaatgggagctgcctctatcacactgaccgtgcaggctagacagctgctgagcggaattgtgcagcagcagagcaacctgctgagagccattgaagcccagcagcacatgctgcagctgaccgtgtggggaatcaaacagctgcaggccagagtgctgaccctggaaagatacgccaaggaccagcagctcctcggcatgtggggatgttctggcaagctgatctgcaccaccaacgtgccctggaacacctcctggtccaacaagagcgagatggacatctggaacaacatgacctggatgcagtgggagagagagatcagcaactacaccgagacaatctacatgctgctcgaggacagccagcggcagcaagagagaaacgagaaggatctgctggccctggactcctggaatagcctgtggaactggttcaatatcaccaactggctgtggtacatcaagatcttcatcatgatcgtcggcggcctgatcggcctgagaatcgtgtttgccgtgctgagcatcgtgaacagagtgcggcagggaatcagcccactgagcctgcaaaccctgacacctaatcctagagagcccgaccggctgagaggcatcgaagaagaaggcggcgagcaggacagagacagatccatcagactggtgtcctgataaggatcc

SEQ ID NO: 68 - DNA, 2109 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacgacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgcacacccctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtggaagtgcagcccctgggaatcgctccaacaggcgctaagagaagagtggtggaacgcgagaaaagagccgctggactgggagccctgtttctgggttttctgggagccgccggaagcacaatgggagctgcctctatcacactgaccgtgcaggctagacagctgctgagcggaattgtgcagcagcagagcaacctgctgagagccattgaagcccagcagcacatgctgcagctgaccgtgtggggaatcaaacagctgcaggccagagtgctgaccctggaaagatacgccaaggaccagcagctcctcggcatgtggggatgttctggcaagctgatctgcaccaccaacgtgccctggaacacctcctggtccaacaagagcgagatggacatctggaacaacatgacctggatgcagtgggagagagagatcagcaactacaccgagacaatctacatgctgctcgaggacagccagcggcagcaagagagaaacgagaaggatctgctggccctggactcctggaatagcctgtggaactggttcaatatcaccaactggctgtggtacatcaagatcttcatcatgatcgtcggcggcctgatcggcctgagaatcgtgtttgccgtgctgagcatcgtgaacagagtgcggcagggatactgataaggatcc

SEQ ID NO: 69 - DNA, 2475 bp, Artificial Sequence
  Description of Artificial Sequence Synthetic polynucleotide
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtggtgccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggctgcggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 70 - DNA, 2469 bp, Artificial Sequence
  Description of Artificial Sequence Synthetic polynucleotide
gtcgacgccaccatgggatctctgcaacctctggccacactgtacctgctgggaatgctggtggcttctgtgctggccgccgagaatctgtgggtcacagtgtactatggcgtgcccgtgtggaaagaggcctgcaccacactgttctgtgccagcgacgccaaggcctacaagaaaaaggtgcggaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagtggttctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgcctcagagcccctgagtgccagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 71 - DNA, 2469 bp, Artificial Sequence
  Description of Artificial Sequence Synthetic polynucleotide
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacatctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactatggtgtgtcctaaactgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtgctgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctctgaacctgacccggaagtctgtgcggatcggccctggccagacattctatgccatgggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagatgatcatcaatatgtggcaaggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 72 - DNA, 2469 bp, Artificial Sequence
  Description of Artificial Sequence Synthetic polynucleotide
gtcgacgccaccatgggatctctgcaacctctggccacactgtacctgctgggaatgctggtggcttctgtgctggccgccgagaatctgtgggtcacagtgtactatggcgtgcccgtgtggaaagaggcctgcaccacactgttctgtgccagcgacgccaaggcctacaagaaaaaggtgcggaacgtctgggccacacactgctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagtggttctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccgggctatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaattgcctcagagcccctgagtgccagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 73 - DNA, 2469 bp, Artificial Sequence
  Description of Artificial Sequence Synthetic polynucleotide
gtcgacgccaccatgggatctctgcaacctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcattgtgcatctgaacacctctgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgagacagaaaccttccggcctgccggcggagacatgagagacaattggagaagcgagctgtacaagtacaaggtggtcaagatcgagcccctgggcgtcgcaccaacacggtgcaagagaagagtcgtgggccgtcgtagaaggcggagagccgttggaattggcgccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggacggcggaggatctggcgacattatcaagctgctgaacgagcaagtgaacaaagagatgaacagctccaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcattagcgccccagagcacaagttcgagggcctgacacagatctttcagaaggcctacgaacacgagcagcacatctccgagagcatcaacaacatcgtggaccacgccattaagagcaaggatcacgccaccttcaattttctgcagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgataaggatcc

SEQ ID NO: 74 - DNA, 2475 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtgcacaacatctgggccacacacgcctgcgtgccaaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaagtgtgccccaagctgaccttcgagcccattcctatccactactgtgcccctgccggctacgccatcctgaagtgcaacaacgagacattcaacggcacaggcccctgcagcaatgtgtccaccgtgcagtgtacccacggcatcagacctgtgctgagcacacagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctggcagtggaaagatacctgagggaccagcagctcctcggaatctggggatgtagcggcaagctgatctgctgcaccaacgtgccctggaacagcagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagctccaacctgtacatgagcatgtccagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 75 - DNA, 2448 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgagggcaatctgtgggtcacagtgtactatggcgtgcccgtgtggaaagaggccaagaccacactgttctgtgccagcgacgccaaggcctacaagaaagaggtgcacaacgtctgggccacacacgcctgtgtgcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgcctgcactcaggcctgtcctaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtcggccggtgtatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtggaagtgcagcccctgggaatcgctcctaccggctgcaagagaagagtggttgaaggcggcggaggatctggcggaggcggatctgctgttggaatcggagctgtgttcctgggctttctgggagccgccggatctacaatgggagctgccagcatgaccctgaccgtgcaggctagaaatctgctgagcggcaaccccgactggctgcctgatatgacagtgtggggaatcaagcagctgcaggccagagtgctggcagtggaaagatacctgagggaccagcagctcctcggaatctggggatgtagcggcaagctgatctgctgcaccaacgtgccctggaactccagctggtccaaccggaatctgagcgagatctgggataacatgacctggctgcagtgggacaaagagatcagcaactacacccagatcatctacggcctgctggaagagagccagaaccagcaagagaaaaacgagcaggacctgctggccctggatggcggaggaagcggagatatcatcaagctgctgaacgagcaagtgaacaaagaaatgaacagcagcaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagatcgagctgatcggcaacgagaaccacggcctgtatctggccgaccagtacgtgaagggaatcgccaagagccggaagtcctgatgaggatcc

SEQ ID NO: 76 - DNA, 2475 bp, Artificial Sequence
  Description of Artificial Sequence Synthetic polynucleotide
gtcgacgccaccatgcctatgggatctctgcagcctctggccacactgtacctgctgggaatgctggtggcttctgtgctggccgccgagaatctgtgggtcacagtgtactatggcgtgcccgtgtggaaagaggccaagaccacactgttctgtgccagcgacgccaaggcctacaagaaagaggtggccaacgtctgggccacacacgcctgtgttcctaccgatccatctcctcaagagctgttcctggaaaacgtgaccgagaacttcaacatgtggaagaacgacatggtggaccagatgcacgaggacatcatcagcctgtgggaccagagcctgaagccttgcgtgaagctgacccctctgtgcgtgaccctgatctgtagcaccgccaccgtgaacaacagagccgtggacgagatgaagaactgcagcttcaacaccaccaccgagatccgggacaagaagaagaaagagtacgccctgttctatcggagcgacgtggtgcccctggacgagacaaacaacaccagcgagtaccggctgatcaactgcaacacctccgccgtgacacaggcttgccccaaagtgaccttcgagcccattcctatccactactgtgcccctgccggctacgccatcctgaagtgcaacaacgagacattcaacggcacaggcccctgcagcaatgtgtccaccgtgcagtgtacccacggcatcagaccagtggtgtctacccagctgctgctgaatggaagcctggccgagaaagaaatcgtgatcagaagcgagaacctgaccaacaacgccaagatcatcatcgtgcacctgaataccagcgtggaaatcgtgtgcacccggcctaacaacaacacccggaagtctgtgcggatcggccctggccagacattctatgccaccggcgatatcatcggcgacatcaagcaggcccactgcaacatcagcgaggaaaagtggaacgagacactgcagaaagtgggcatcgagctgcagaagcacttccccaacaagaccatcaagtacaaccagagcgctggcggcgacatggaaatcaccacacacagcttcaattgtggcggcgagttcttctactgcaataccagcaagctgttcaacagcacctacaacggcacctatatcagcaccaactccaccaatagcaccagctacatcaccctgcagtgccggatcaagcagatcatcaatatgtggcaaggcgtgggcagagctatgtacgcccctcctatcgccggcaacatcacctgtcggagcaatatcacaggcctgctgctcaccagagatggcggcatcaacaacgtgtccaacgaaaccgaaaccttccggcctgccggcggagacatgagagacaattggagaagcgagctgtacaagtacaaggtggtcaagatcgagcccctgggcgtcgcaccaacacggtgcaagagaagagtcgtgggacgtagacgaaggcggagagccgttggaatcggagccgtgttcctgggctttctgggagccgctggatctacaatgggcgctgccagcatgaccctgacagtgcaggctagaaatctgctgagcggcatcgtgcagcagcagagcaatctgctcagagcccctgaggctcagcagcacctcctgaaactgacagtgtggggaatcaagcagctgcaggccagagtgctgaccgtggaaagatacgccagagatcagcagctcctcggaatctggggctgtagcggcaagctgatctgctgcaccaacgtgccctggaactccagctggtccaaccggaatctgagcgagatctgggataacatgacctggctgcagtgggacaaagagatctccaactacacccagatcatctacggcctgctggaagagagccagaaccagcaagagaaaaacgagcaggacctgctggccctggatggcggaggatctggcgacattatcaagctgctgaacgagcaagtgaacaaagaaatgaacagcagcaacctgtacatgagcatgagcagctggtgttacacccacagccttgatggcgccggactgttcctgtttgatcacgccgccgaggaatacgagcacgccaagaagctgatcatcttcctgaacgagaacaatgtgcccgtgcagctgaccagcatctctgcccctgagcacaagttcgagggcctgacacagatctttcagaaggcctacgaacacgagcagcacatctccgagtccatcaacaatatcgtggaccacgccattaagagcaaggatcacgccaccttcaactttctccagtggtacgtggccgaacagcacgaggaagaagtgctgttcaaggacatcctggacaagattgagctgatcggcaacgagaaccacggcctgtatctggccgaccagtacgtgaagggaatcgccaagagccggaagtcctgatgaggatcc

SEQ ID NO: 77 - protein, 733 aa, Artificial Sequence
  Description of Artificial Sequence Synthetic polypeptide
MPMGSLQPLATLYLLGMLVASVLAEGNLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLTLERYAKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGISPLSLQTLTPNPREPDRLRGIEEEGGEQDRDRSIRLVS

SEQ ID NO: 78 - protein, 695 aa, Artificial Sequence
  Description of Artificial Sequence Synthetic polypeptide
MPMGSLQPLATLYLLGMLVASVLAEGNLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNDETFNGTGPCSNVSTVQCTHGIRPVVSTQLLLNGSLAEKEIVIRSENLTNNAKIIIVHLHTPVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVEVQPLGIAPTGAKRRVVEREKRAAGLGALFLGFLGAAGSTMGAASITLTVQARQLLSGIVQQQSNLLRAIEAQQHMLQLTVWGIKQLQARVLTLERYAKDQQLLGMWGCSGKLICTTNVPWNTSWSNKSEMDIWNNMTWMQWEREISNYTETIYMLLEDSQRQQERNEKDLLALDSWNSLWNWFNITNWLWYIKIFIMIVGGLIGLRIVFAVLSIVNRVRQGY

SEQ ID NO: 79 - protein, 817 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWCQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGCGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 80 - protein, 815 aa, Artificial Sequence
  Description of Artificial Sequence Synthetic polypeptide
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 81 - protein, 815 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNIWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTMVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPLNLTRKSVRIGPGQTFYAMGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKMIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 82 - protein, 815 aa, Artificial Sequence
  Description of Artificial Sequence Synthetic polypeptide
MGSLQPLATLYLLGMLVASVLAAENLWVTVYYGVPVWKEACTTLFCASDAKAYKKKVRNVWATHCCVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQWFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNCLRAPECQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 83 - protein, 815 aa, Artificial Sequence
  Description of Artificial Sequence Synthetic polypeptide
MGSLQPLATLYLLGMLVASVLAAENLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 84 - protein, 817 aa, Artificial Sequence
  Description of Artificial Sequence Synthetic polypeptide
MPMGSLQPLATLYLLGMLVASVLAAENLWVTVYYGVPVWKEAKTTLFCASDAKAYKKEVHNIWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQVCPKLTFEPIPIHYCAPAGYAILKCNNETFNGTGPCSNVSTVQCTHGIRPVLSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 85 - protein, 808 aa, Artificial Sequence
  Description of Artificial Sequence Synthetic polypeptide
MPMGSLQPLATLYLLGMLVASVLAEGNLWVTVYYGVPVWKEAKTTLFCASDAKAYKKEVHNVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSACTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRCMYAPPIAGNITCRSNITGLLLTRDGGINNVSNETETFRPAGGDMRDNWRSELYKYKVVEVQPLGIAPTGCKRRVVEGGGGSGGGGSAVGIGAVFLGFLGAAGSTMGAASMTLTVQARNLLSGNPDWLPDMTVWGIKQLQARVLAVERYLRDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 86 - protein, 817 aa, Artificial Sequence
  Description of Artificial Sequence Synthetic polypeptide
MPMGSLQPLATLYLLGMLVASVLAAENLWVTVYYGVPVWKEAKTTLFCASDAKAYKKEVANVWATHACVPTDPSPQELFLENVTENFNMWKNDMVDQMHEDIISLWDQSLKPCVKLTPLCVTLICSTATVNNRAVDEMKNCSFNTTTEIRDKKKKEYALFYRSDVVPLDETNNTSEYRLINCNTSAVTQACPKVTFEPIPIHYCAPAGYAILKCNNETFNGTGPCSNVSTVQCTHGIRPVVSTQLLLNGSLAEKEIVIRSENLTNNAKIIIVHLNTSVEIVCTRPNNNTRKSVRIGPGQTFYATGDIIGDIKQAHCNISEEKWNETLQKVGIELQKHFPNKTIKYNQSAGGDMEITTHSFNCGGEFFYCNTSKLFNSTYNGTYISTNSTNSTSYITLQCRIKQIINMWQGVGRAMYAPPIAGNITCRSNITGLLLTRDGGINNVSNETETFRPAGGDMRDNWRSELYKYKVVKIEPLGVAPTRCKRRVVGRRRRRRAVGIGAVFLGFLGAAGSTMGAASMTLTVQARNLLSGIVQQQSNLLRAPEAQQHLLKLTVWGIKQLQARVLTVERYARDQQLLGIWGCSGKLICCTNVPWNSSWSNRNLSEIWDNMTWLQWDKEISNYTQIIYGLLEESQNQQEKNEQDLLALDGGGSGDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 87 - protein, 4 aa, Artificial Sequence
  Description of Artificial Sequence Synthetic peptide
VDTA

SEQ ID NO: 88 - protein, 4 aa, Unknown
  Description of Unknown cleavage site sequence
REKR

SEQ ID NO: 89 - protein, 9 aa, Unknown
  Description of Unknown cleavage site sequence
RRVVEREKR

SEQ ID NO: 90 - protein, 9 aa, Unknown
  Description of Unknown cleavage site sequence
ERVVEREKE

SEQ ID NO: 91 - protein, 4 aa, Unknown
  Description of Unknown cleavage site sequence
SEKS

SEQ ID NO: 92 - protein, 79 aa, Unknown
  Description of Unknown envelope sequence
MRVMGIQRNYPQWWIWSMLGFWMLMICNGMWVTVYYGVPVWKEAKTTLFCASDAKAYEKEVHNVWATHACVPTDPNPQE

SEQ ID NO: 93 - protein, 24 aa, Homo sapiens
MPMGSLQPLATLYLLGMLVASVLA

SEQ ID NO: 94 - protein, 5 aa, Unknown
  Description of Unknown recognition motif sequence
  MOD_RES (3)..(3): Any amino acid
LPXTG

SEQ ID NO: 95 - protein, 5 aa, Artificial Sequence
  Description of Artificial Sequence Synthetic peptide
LPSTG

SEQ ID NO: 96 - protein, 6 aa, Artificial Sequence
  Description of Artificial Sequence Synthetic peptide
GGGGGG